(19) United States
(12) Patent Application Publication
(10) Pub. No.: US 2014/0348819 A1
(43) Pub. Date: Nov. 27, 2014

(54) Methods of Treating Cancer

(71) Applicants: Todd Golub, Newton, MA (US); Ravid Straussman, Brookline, MA (US)

(72) Inventors: Todd Golub, Newton, MA (US); Ravid Straussman, Brookline, MA (US)

(73) Assignees: Dana-Farber Cancer Institute, Inc., Boston, MA (US); The Broad Institute, Inc., Cambridge, MA (US)

(21) Appl. No.: 14/128,916

(22) PCT Filed: Jun. 22, 2012
(86) PCT No.: PCT/US12/43788

§ 371 Date: Aug. 19, 2014

Related U.S. Application Data
(60) Provisional application No. 61/501,091, filed on Jun. 24, 2011.

(51) Int. Cl. A61K 39/395; A61K 31/166; C12Q 1/68; A61K 31/437; G01N 33/574; A61K 31/4545; A61K 45/06

(52) U.S. Cl. CPC A61K 39/3955; A61K 31/4545; A61K 31/166; A61K 45/06; A61K 31/437; G01N 33/574; C12Q 1/68; A61K 2039/505
USPC 424/133.1; 424/158.1; 514/326; 514/619; 514/300; 435/7.92; 435/7.23; 435/6.11

(57) ABSTRACT

The present invention provides methods of treating cancer.

### RELATED APPLICATIONS

This application claims the benefit of U.S. Provisional Application No. 61/501,091 filed Jun. 24, 2011 the contents of which is incorporated herein by reference in its entirety.

### GOVERNMENT INTEREST

This invention was made with government support under P50CA093683 awarded by the National Institutes of Health and U54CA112962 awarded by the National Institutes of Health. The government has certain rights in the invention.

### FIELD OF THE INVENTION

The present invention relates generally to the treatment of cancer. More specifically the invention related to preventing or reducing chemoresistance in a tumor by administering to a cancer patient a chemotherapeutic agent together with another agent that blocks the activity of Hepatocyte Growth Factor (HGF) or its cognate receptor c-MET.

### BACKGROUND OF THE INVENTION

Cancer is one of the leading causes of death. Although it has been the focus of medical research for a long period of time, the main cancer therapies to date remain to be surgery, radiation therapy and chemotherapy. Each one of these therapies is subject to limitations which are not currently overcome, and the search for an improved therapy continues.

One significant problem of chemotherapy is that tumors can develop resistance to drugs. For example, a drug may be highly effective when it is first introduced to the patient, killing tumor cells and reducing the size of the tumor such that the patient goes into a remission. However, the tumor may regrow after a period of time, and this time the same drug is not effective at all at killing the regrown tumor cells. This phenomenon of acquired resistance is believed to be due to a small population of drug resistant cells in the tumor which survives the initial drug treatment while the majority of the tumor is killed. These resistant cells eventually grow back to form a tumor comprising essentially only drug resistant cells.

Many patients also have some extent of primary or innate resistance to chemotherapy, where primary or innate resistance refers to the phenomenon where a tumor exhibits resistance to a chemotherapeutic agent prior to any exposure to or treatment with the chemotherapeutic agent. Indeed, complete clinical responses are rare, suggesting that mechanisms exist to render a substantial portion of tumor cells resistant to treatment. For example, melanomas harboring the V600E mutation show a dramatic response to RAF inhibitors, but responses are almost always partial, and tumors often recur within a couple of months. As genetic changes that are known to be responsible for chemoresistance are only rarely found in pre-treatment tumors, such mutations cannot fully explain the extent of innate resistance seen in patients.

Treatment at the outset with a combination of drugs was proposed as a solution for acquired drug resistance, given the small probability that mutations which lead to two or more different drug resistance pathways would arise spontaneously in the same cell (DeVita, Jr., 1983). However, it has been discovered that cells which are resistant to one drug are often resistant to multiple drugs, including structurally unrelated drugs which are capable of killing tumor cells by different pathways. Therefore, known combination drug therapies do not solve the problem. The mechanisms behind innate drug resistance are even more elusive and as such are even harder to tackle.

Therefore, the causes of both innate and acquired drug resistance are not fully understood and there is still a need for methods to overcome drug resistance in order to treat tumors more effectively.

### SUMMARY OF THE INVENTION

The invention features methods of preventing or reducing chemoresistance in a tumor comprising administering to a cancer patient a one or more chemotherapeutic agent and a c-MET kinase (MET) inhibitor. In some aspects the chemoresistance is stromal cell mediated. The tumor comprises a B-RAF activating mutation.

The invention further features methods of treating a tumor in a subject having a B-RAF activating mutation by administering an effective amount of a MET inhibitor.

The MET inhibitor a small molecule or neutralizing antibody that inhibits MET activity. For example, the MET inhibitor is a hepatocyte growth factor (HGF) neutralizing antibody like Ficlatuzumab. For example, the MET inhibitor is a MET neutralizing antibody. Alternatively, the MET inhibitor is a small molecule that inhibits HGF or MET. In other embodiments, the MET inhibitor is (3Z)-5-(2,3-dihydro-1H-indol-1-ylsulfonyl)-3-({3,5-dimethyl-4-[(4-methylpiperazin-1-yl)carbonyl]-1H-pyrrol-2-yl}methylene)-1,3-dihydro-2H-indol-2-one, (3Z)-N-(3-chlorophenyl)-3-({3,5-dimethyl-4-[(4-methylpiperazin-1-yl)carbonyl]-1H-pyrrol-2-yl}methylene)-N-methyl-2-oxoindoline-5-sulfonamide, (3Z)-N-(3-chlorophenyl)-3-{[3,5-dimethyl-4-(3-morpholin-4-ylpropyl)-1H-pyrrol-2-yl]methylene}-N-methyl-2-oxoindoline-5-sulfonamide, AMG-208, AMG-337, Axitinib, Foretinib, JNJ-38877605, MGCD-265, PF-04217903, Crizotinib, Cabozantinib, PHA-665752, SGX-523, SU11274, XL184, ARQ197, XL880, INC280 or Onartuzumab (MetMab), Trametinib, selumetinib, PD0325901, PD184.352, PHA-665752, JNJ-38877605, Rilotumumab or Ficlatuzumab.

The chemotherapeutic agent is a RAF inhibitor (e.g., Vemurafenib or Dabrafenib), a MEK inhibitor, a PI3K inhibitor, an AKT inhibitor or a combination thereof. For example, the RAF inhibitor is a B-RAF inhibitor. For example, the chemotherapeutic agent is a RAF inhibitor and a MEK inhibitor.

The tumor is refractory to the first chemotherapeutic agent(s) when administered alone. The tumor is, for example, a melanoma, colon cancer, lung cancer, brain cancer, thyroid cancer or a hematologic cancer.

In some aspects the subject has been previously exposed to one or more chemotherapeutic agents. The MET inhibitor is administered concurrently with the chemotherapeutic agent. Alternatively, the MET inhibitor is administered prior to administration of the chemotherapeutic agent.

The MET inhibitor is administered into or near the tumor or systemically.

Also include in the invention are methods of diagnosing or determining a predisposition to developing chemoresistance in a tumor by determining the level of HGF in expression in the tumor and comparing the level of HGF expression to a control sample. An increase level of HGF expression in the tumor compared to the control indicates chemoresistance or a predisposition to developing chemoresistance in the tumor.

The invention further provides methods of diagnosing or determining a predisposition to developing chemoresistance in a tumor by determining the level of MET activation in the tumor and comparing the level of MET activation to a control sample. An increase level of MET activation in the tumor compared to the control indicates chemoresistance or a predisposition to developing chemoresistance in the tumor. In some aspects the tumor has a BRAF activating mutation.

The levels of HGF expression is determined detecting HGF polypeptide, or HGF nucleic acid (e.g., DNA or RNA). MET activation is determined by detecting MET phosphorylation.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

Other features and advantages of the invention will be apparent from and encompassed by the following detailed description and claims.

### BRIEF DESCRIPTION OF THE DRAWINGS

FIG. 1A-1B shows the effect of chemotherapy on cancer cell lines proliferation without or with the presence of stromal cells.

FIG. 2A-2B shows that a subset of stromal cell lines can induce drug resistance. FIG. 2B shows that while melanoma cell lines (represented here by G-361) are sensitive to the BRAF inhibitor (BRAFi) PLX4720 and become resistant only in the presence of a subset of stromal cells, colorectal cancer cell lines (represented here by WiDr) are largely not sensitive to BRAFi with or without stromal cells.

FIG. 3A-3C is fluorescent images that show the co-culture system of melanoma cell lines and the fibroblasts. The fibroblasts allow the melanoma cells to proliferate under continuous drug treatment.

FIG. 4 shows that the resistance is mediated by a secreted factor. Stromal pre-conditioned media (PCM) is sufficient to confer drug resistance.

FIG. 5A shows the effect of the addition of 22 recombinant receptor tyrosine kinase ligands to 6 melanoma cell lines. HGF is the only one that can substantially confer drug resistance to the cell lines.

FIG. 5B shows screening results of the secreted factors. Two protein array systems were used to screen more than 500 known secreted proteins. HGF shows a high expression level in a subset of 6 cell lines. The figure also shows that the 6 stromal cell lines that secret HGF are the same stromal cell lines that confer resistance to melanoma cell lines.

FIG. 6A shows that when correlating 274 secreted factors with the stromal rescue, HGF gets the highest correlation score. FIG. 6B shows the correlation between HGF secretion and resistance.

FIG. 7A-7B shows that recombinant HGF can confer BRAFi (PLX4720) and MEKi (MEK inhibitor, PD184352) resistance to melanoma cell lines.

FIG. 8 shows that anti-HGF neutralizing antibodies abolish the stromal cells rescue effect.

FIG. 9A-9B shows the synergistic effect of Crizotinib. Specifically (B) shows that no substantial synergistic effect in DLD-1 cells was found. These colorectal cells do not harbor the BRAF activating mutation and are not sensitive to BRAF inhibition. The DLD-1 cells do harbor the BRAF activating mutation V600E but are not sensitive at all to BRAF inhibition. They actually grow faster under BRAFi. The addition of Crizotinib, which blocks MET, restores full sensitivity of this colorectal cancer cell line to BRAFi.

FIG. 10A shows the activation of pERK by RTK ligands under PLX4720. FIG. 10B shows the activation of pAKT by RTK ligands under PLX4720. HGF activates AKT in every cell lines tested.

FIG. 11A-11B shows that HGF can substantially reactivate pERK under BRAFi (PLX4720) but not under MEKi (PD184352) and FIG. 11C shows that HGF can confer better resistance to BRAFi than MEKi.

FIG. 12 shows that HGF confers resistance to the combination of BRAFi and AKTi (AKT inhibitor, MK-2206), but not to the combination of MEKi and AKTi.

FIG. 13 shows that activation of pAKT (phosphorylated AKT) by HGF under BRAFi (PLX4720) is sustained over time.

FIG. 14 shows the current working model.

FIG. 15 shows the immunohistochemistry staining of anti-MET antibodies in a skin melanoma sample.

FIG. 16A-16B shows the immunohistochemistry staining of anti-phospho-MET antibodies in a metastatic melanoma sample.

FIG. 17 A-E shows IHC staining for pMET of biopsies from melanoma. “Ala” biopsy was taken 1 week before treatment while “A2a” was taken 10 days after treatment with BRAFi (PLX4720).

FIG. 18 shows the western blot result of phospho-MET in multiple melanoma cell lines and colorectal cancer cell lines.

FIG. 19 shows the immunohistochemistry staining of anti-phospho-MET antibodies in a colon cancer sample.

FIG. 20A-20B shows the immunohistochemistry staining of anti-MET antibodies in a colon cancer sample.

FIG. 21 shows the immunohistochemistry staining of anti-phospho-MET antibodies in MKN45 cells treated with or without the MET inhibitor (SU11274).

FIG. 22 shows that HGF is present in the stromal cells of melanoma and correlates with poor response to therapy. FIG. 22A shows the immunohistochemistry (IHC) analysis of HGF expression in pre-treatment melanoma section from patient #32. Black arrow in left panel: normal epidermis. Top arrow in right panel: tumor cells. Lower arrow in right panel: HGF-expressing stroma. FIG. 22B shows the HGF expression by IHC analysis from melanoma sections from patient #23. Left panel: pre-treatment biopsy. Middle panel: On treatment biopsy (2 weeks after the initiation of treatment with the BRAFi Vemurafenib (PLX4032) and one month after the pre-treatment biopsy was obtained). Right panel: Progression (12 months after the initiation of treatment). FIG. 22C shows the maximal response to treatment of BRAF V600E melanoma patients with or without stromal HGF as measured by IHC. Patients with stromal HGF had a significantly poorer response to treatment compared to those lacking expression (*P<0.05 by two-sample t-test assuming equal variance). Median values for each group are depicted above the median line.

FIG. 23 shows the characterization of molecular mechanism of HGF-induced primary resistance. FIG. 23A shows the levels of phosphorylated ERK (T202/Y204) 1 hour after treatment with media (−) or with 22 cytokines in the presence of BRAFi (PLX4720) or DMSO (DM) control. FIG. 23B is a western blot showing the activation of AKT (phosphorylated AKT, S473) after 1 hour and 24 hours after treatment with HGF, IGF-1 (IGF), and Insulin (INS) in the presence of BRAFi (PLX4720, 2 μM). FIG. 23C is a western blot showing the effect of HGF (25 ng/mL) on MAPK and PI3K/AKT pathway activation (pRAF1, pMEK, pERK, pAKT and pMET) in melanoma cell lines after 24 hours of treatment with 2 μM BRAFi (PLX4720) or 2 μM MEKi (PD184352).

FIG. 24 shows levels of HGF in media from stromal cell lines and BRAF V600E mutated cancer cell lines as detected by ELISA analysis. CRC=Colorectal cancer. GBM=Glioblastoma multiforme.

FIG. 25 shows the expression of MET and pMET in melanomas by IHC (25 a-c) or Immunofluorescence analysis. FIG. 25A-25C shows MET levels in sections from patient #33 (pre-treatment, (A)), #25 (on treatment, (B)), and #27 (on treatment, (C)). FIGS. 25D-25F shows levels of phospho-MET by immunofluorescence. Sections represented are from patient #1 (on treatment,(D)), #18 (on treatment,(E)), and #27 (on treatment, (F)). Patient #1 had a complete response to therapy and was negative for stromal HGF while patients #18 and #27 had partial responses and were positive for stromal HGF.

FIG. 26 shows tissue micro array (TMA) analysis for stromal HGF. FIG. 26A is a summary table of TMA sections analyzed for stromal HGF expression by IHC. FIG. 26B shows HGF expression levels in normal skin and melanoma stromal cell. Arrows point to stromal HGF. FIG. 26C shows that HGF expression was negative in normal colon and positive in colorectal cancer (BRAF V600E) sections.

FIG. 27 shows the effect of 50 ng/mL HGF on sensitivity to BRAFi (PLX4720 and Vemurafinib) in melanoma cell lines. Results were normalized to no drug and averaged. Bars represent standard error between replicates (n=3).

FIG. 28 shows the proliferation of melanoma cell lines that were co-cultured with stromal cell lines and treated with BRAFi (PLX4720, 2 μM) or MEKi (PD184352, 1 μM) with or without 0.2 μM of Crizotinib. Proliferation was quantified after 7 days and normalized to non-treated cells. Bars represent standard error between replicates (n=3).

FIG. 29 shows the correlation between MET expression and HGF-mediated resistance to (BRAFi) PLX4720. FIG. 29A shows the IC50s of 27 V600E BRAF melanoma cell lines, generated from a 10-point PLX4720 concentration range using CellTiter Glo readout after 72 hours of treatment. IC50s>10 are represented as 10. FIG. 29B shows 20 cell lines (with IC50s>6 uM from (A)) were treated with or without the presence of 50 ng/ml HGF. The calculated IC50s +HGF divided by the IC50s −HGF are represented. Ratios>15 are represented as 15. FIG. 29C shows the levels of total MET in the 20 selected cell lines before or 24 hours after treatment with 2 μM of PLX4720. FIG. 29D shows the correlation between PLX4720 IC50 (+HGF/−HGF) (B) and c-MET expression (C).

FIG. 30 shows the expression of receptor tyrosine kinases (RTKs) in melanoma and colorectal cancer cell lines by Western blot analysis.

FIG. 31A-B shows the activation of RTKs by the addition of different RTK ligands to melanoma cell lines. Activation of all relevant RTKs was measured using high throughput tyrosine kinase phosphorylation profiling.

FIG. 32 shows the effect of stromal PCM on MAPK and PI3 K pathway activation under BRAFi (PLX4720, 2 μM) treatment. MAPK and PI3K/AKT pathway activation was assessed after 24 hours of treatment by immunoblot analysis of MET, AKT, MEK, ERK and their respective phosphorylation status. Black names: PCM from stromal cells that do not rescue the melanoma cell lines from PLX4720. Red names: PCM from stromal cells that can rescue.

FIG. 33 shows the synergistic effect of BRAFi/METi in BRAF V600E colorectal and glioma cell lines. FIG. 33A shows the levels of c-MET and phosphorylated MET in colorectal cancer (CRC) and glioma cell lines (upper panel). Lower panel: The effect of combinations of 5 concentrations of METi (Crizotinib) and 8 concentrations of BRAFi (PLX4720) were measured by CellTiter Glo after 72 hours of treatment. Excess above BLISS for measuring synergistic effect was calculated. FIGS. 33B-33D are graphic representations of excess above BLISS for 3 of the cell lines (RKO, HCT-116, KG-1-C).

FIG. 34 shows the BRAFi/METi synergistic effect in BRAF V600E compared to BRAFi/EGFRi synergistic effect in RKO (CRC) and KG-1-C (glioma) cell lines. The effect of combinations of 5 concentrations of METi (Crizotinib/Gefitinib) and 4 concentrations of BRAFi (PLX4720) were measured by GFP at day 7. Excess above BLISS for measuring synergistic effect was calculated.

FIG. 35 shows the effect of METi, Crizotinib (0.2 μM), on the treatment of the colorectal cancer cell line RKO and the glioblastoma cell line KG-1-C with 2 μM PLX4720 or 1 μM PD184352. FIG. 35A is a western blot showing MAPK and PI3K/AKT pathway activation after 24 hours of treatment (pMEK, pERK, pAKT and pMET). FIG. 35B shows the normalized proliferation of the cell lines under the same conditions is represented. Bars represent standard error between replicates (n=4).

### DETAILED DESCRIPTION OF THE INVENTION

This invention is based upon the discovery that hepatocyte growth factor (HGF) induces primary or innate drug resistance in cancer cells and that this resistance is reversed by the addition of HGF neutralizing antibodies or a c-MET inhibitor (e.g. small molecule or an anti-MET antibody). This finding indicates that treatment with a MET inhibitor, including HGF neutralizing antibodies, would provide therapeutic benefits to cancers that have an activating mutation in BRAF.

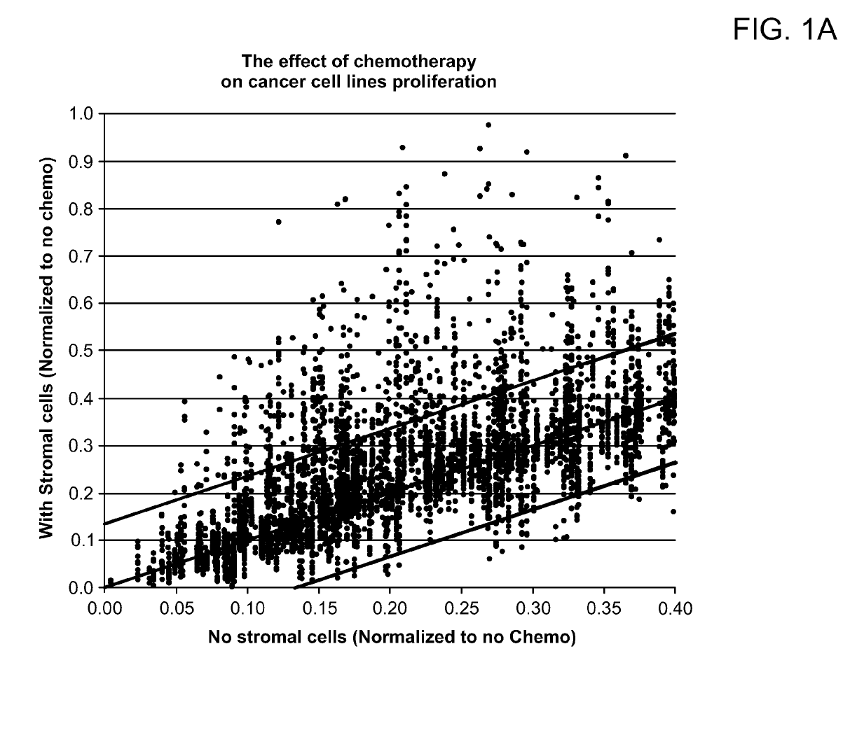

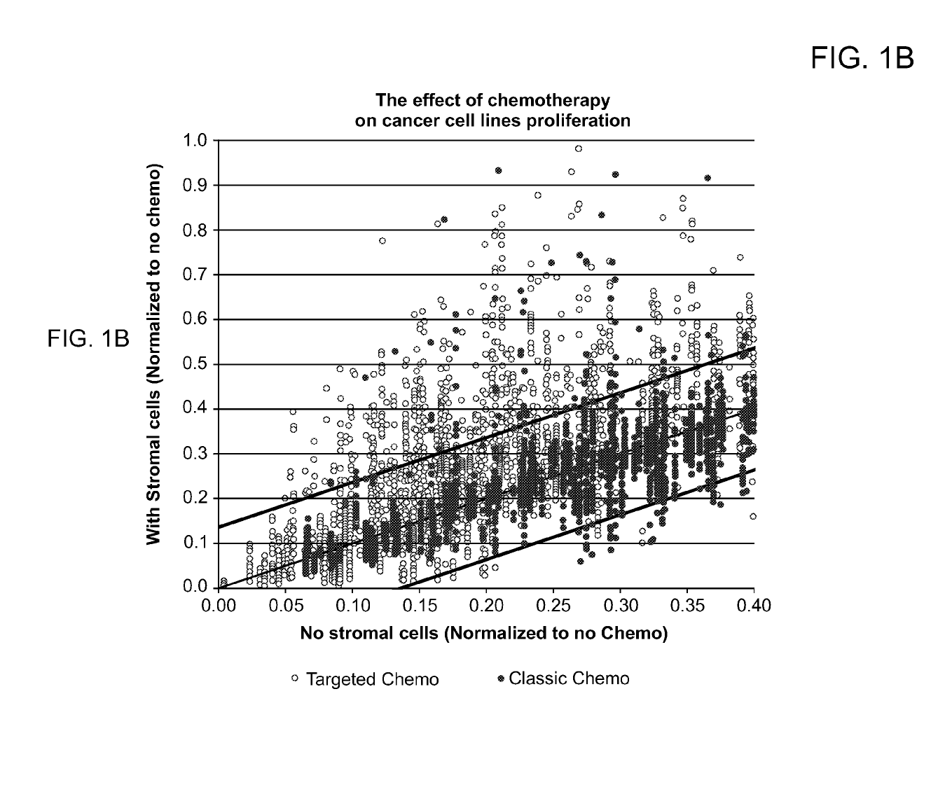

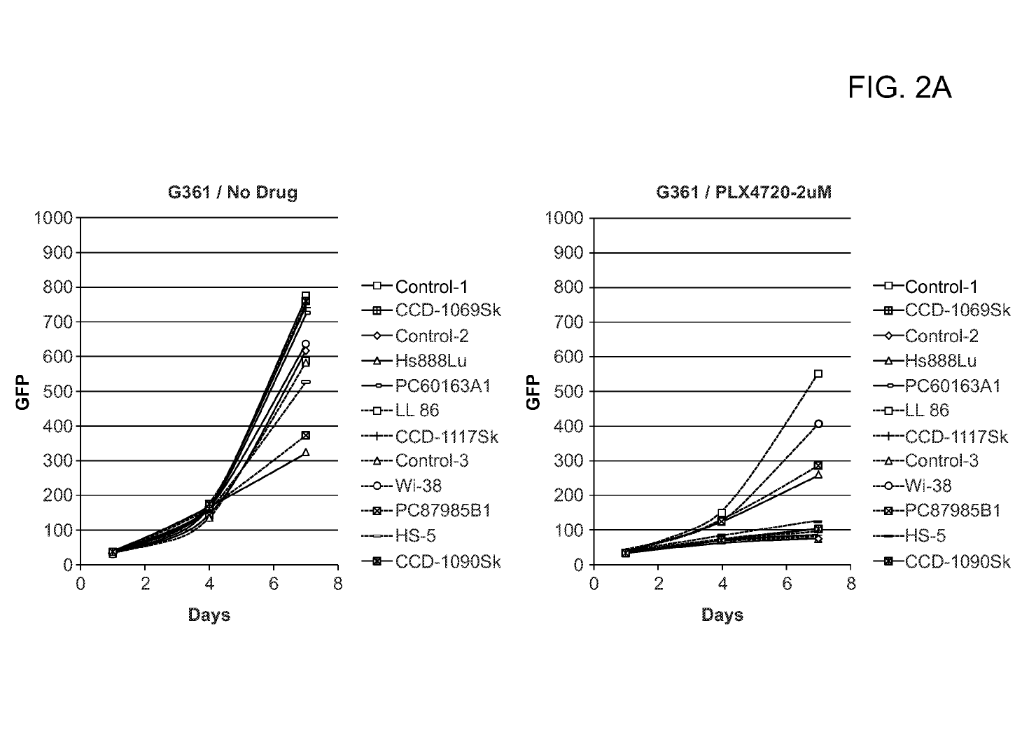

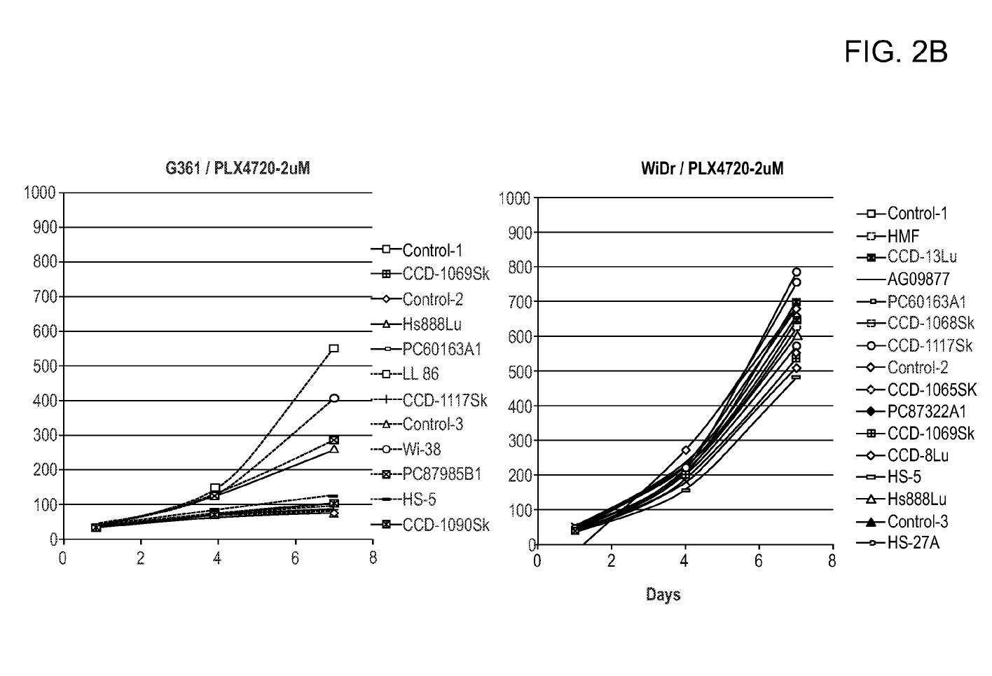

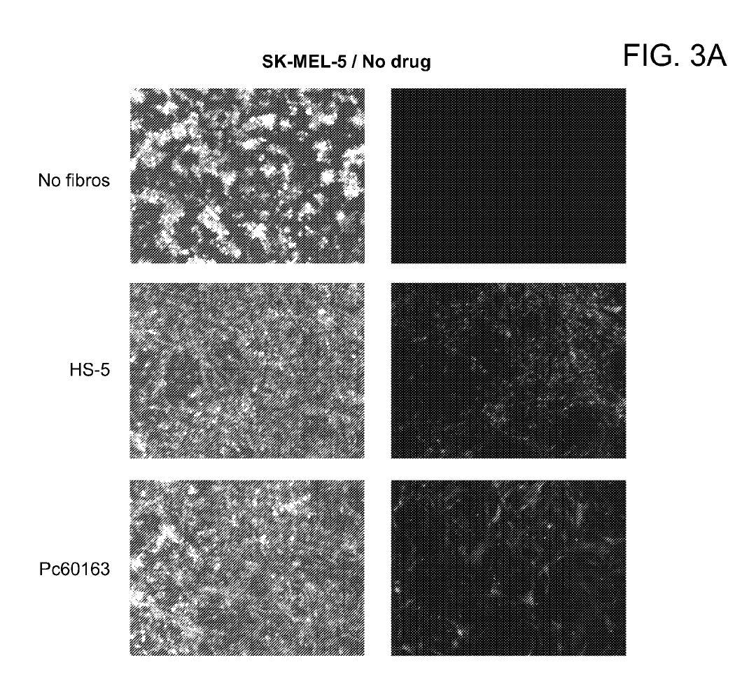

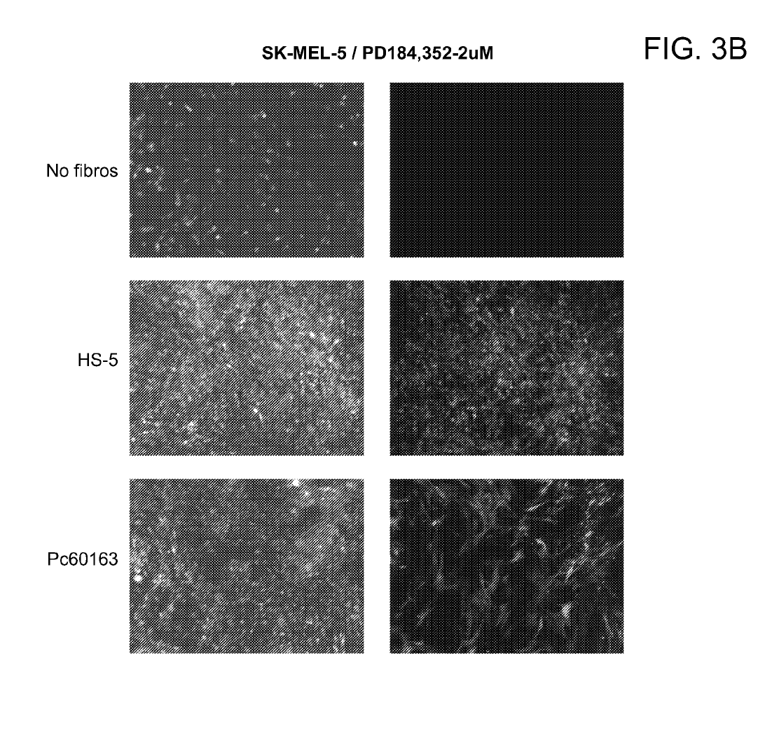

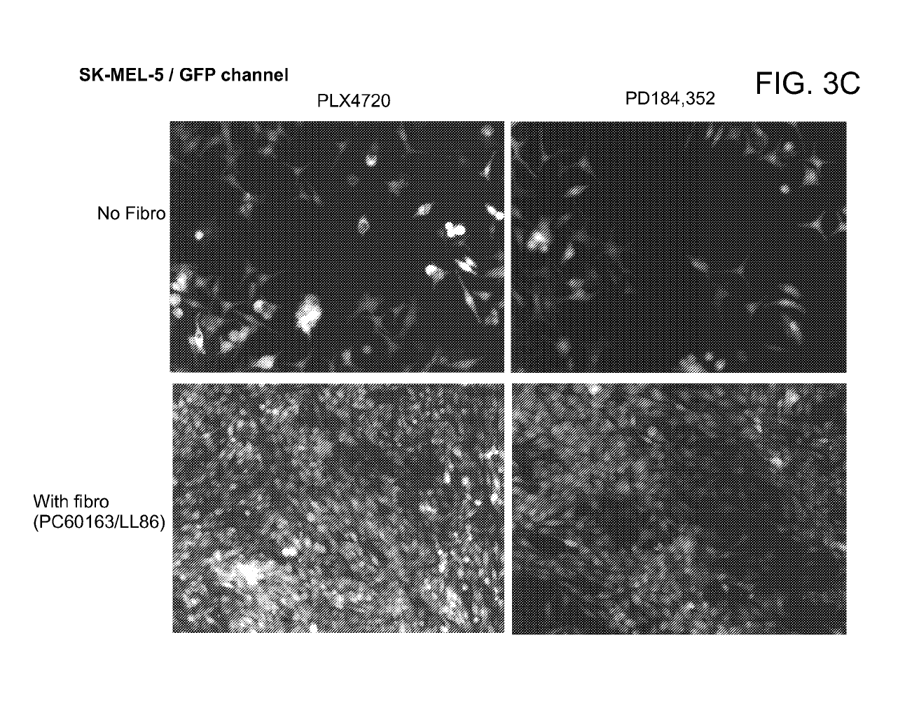

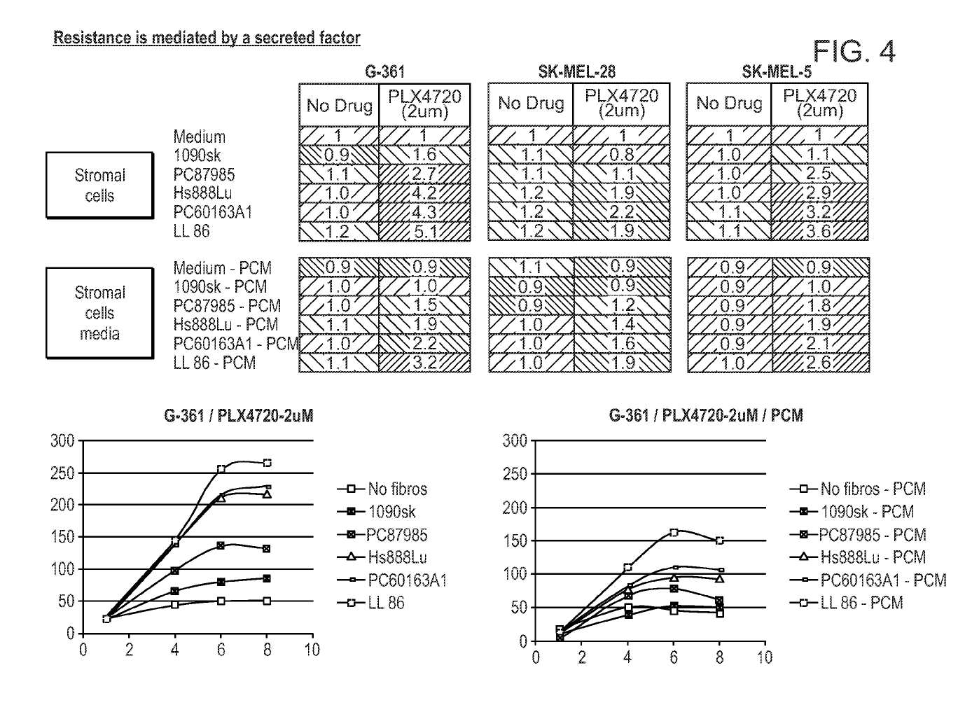

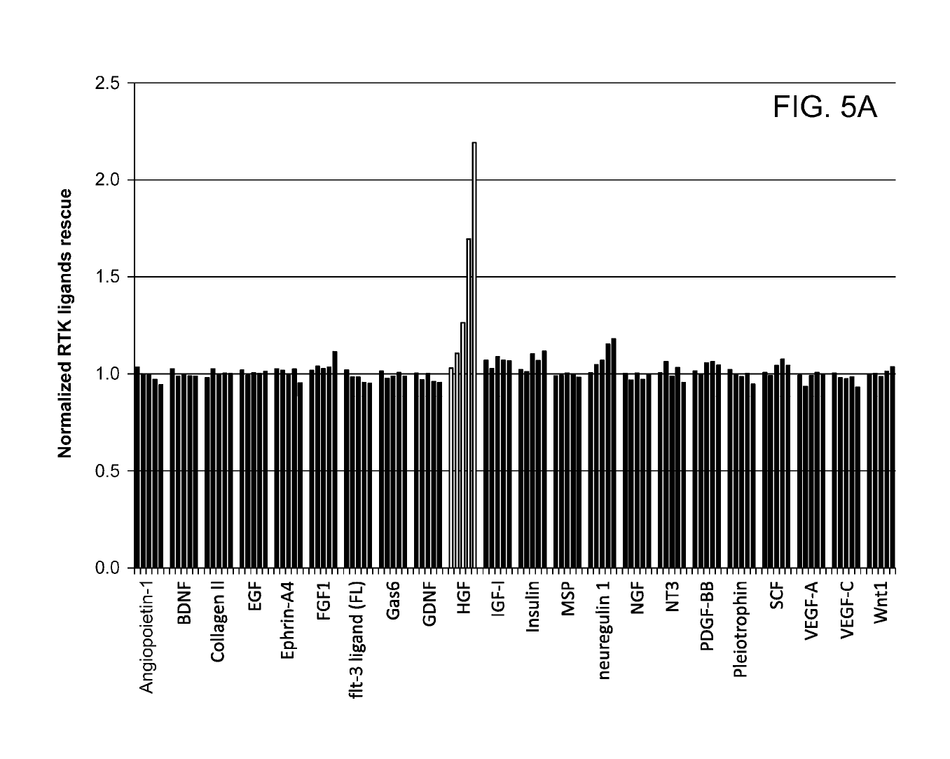

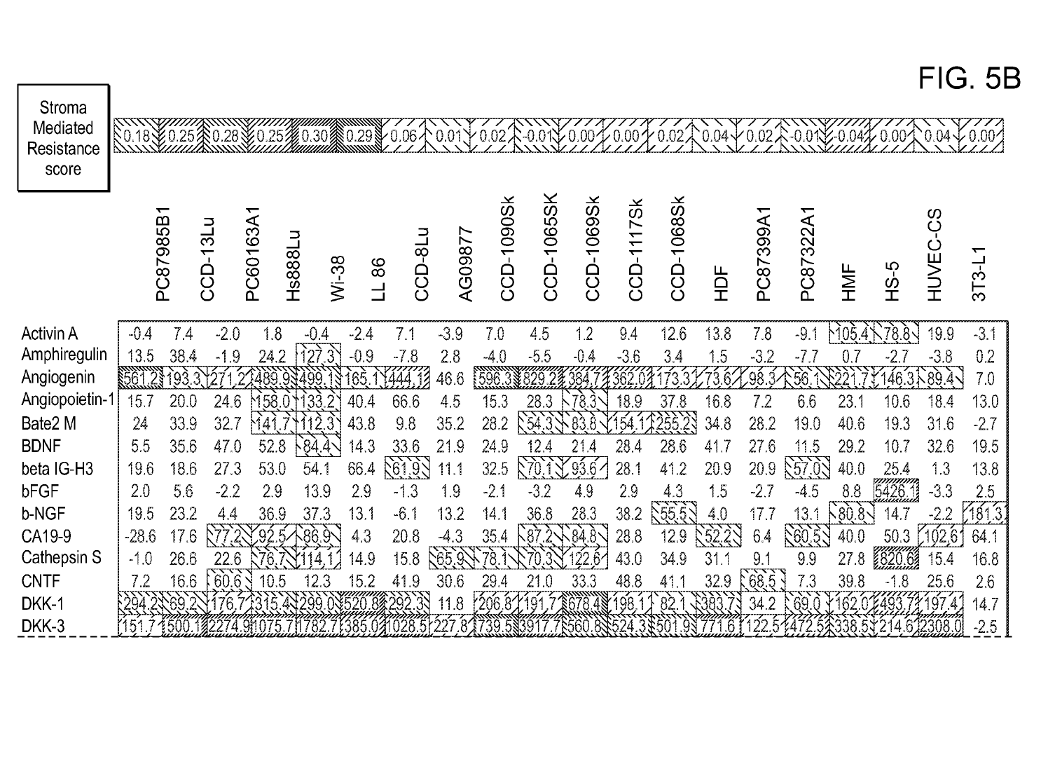

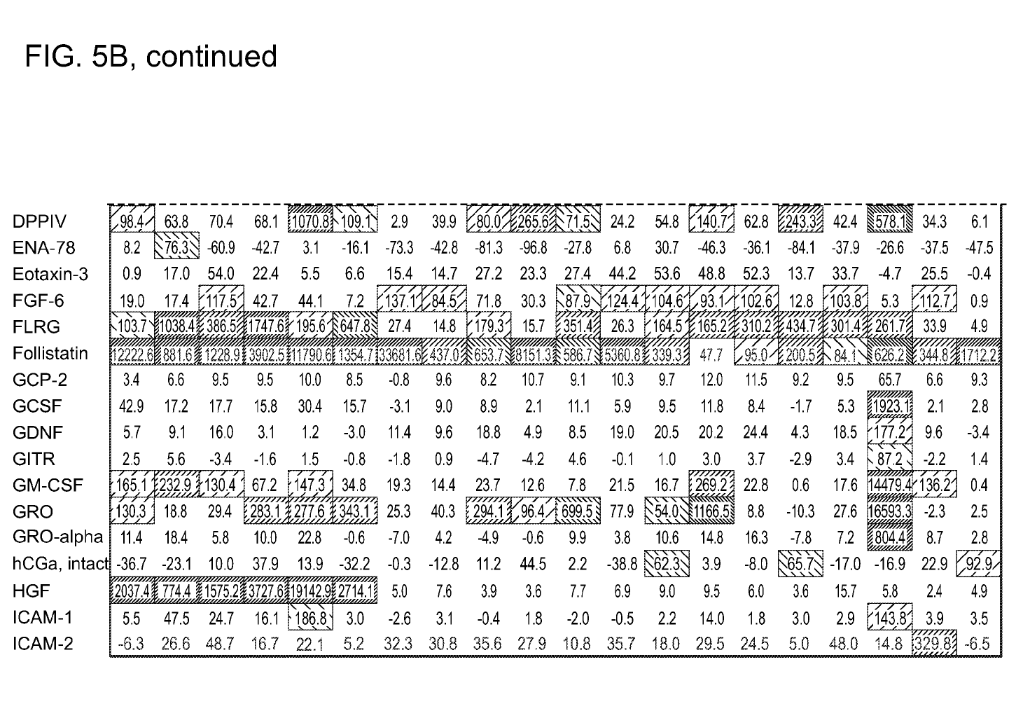

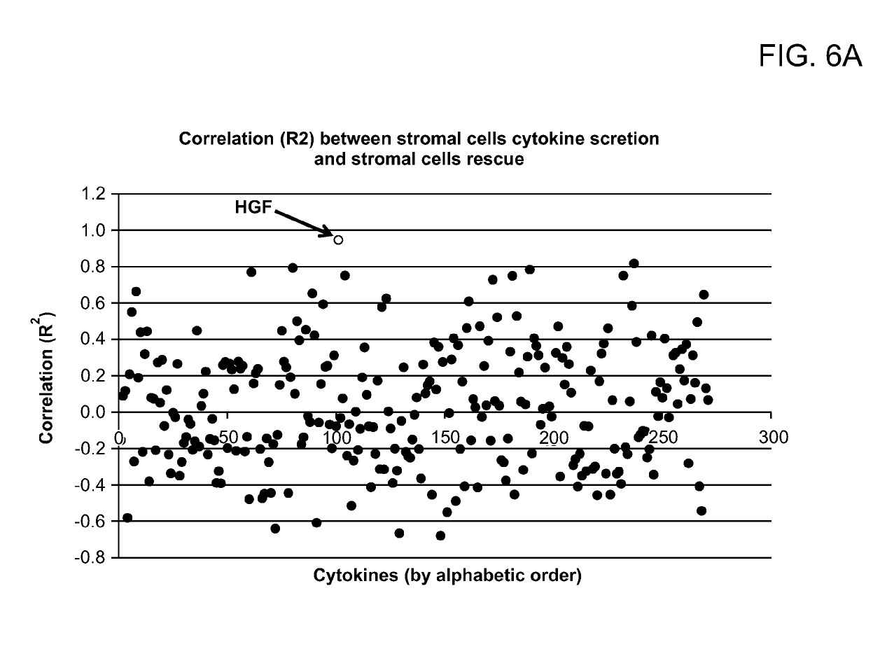

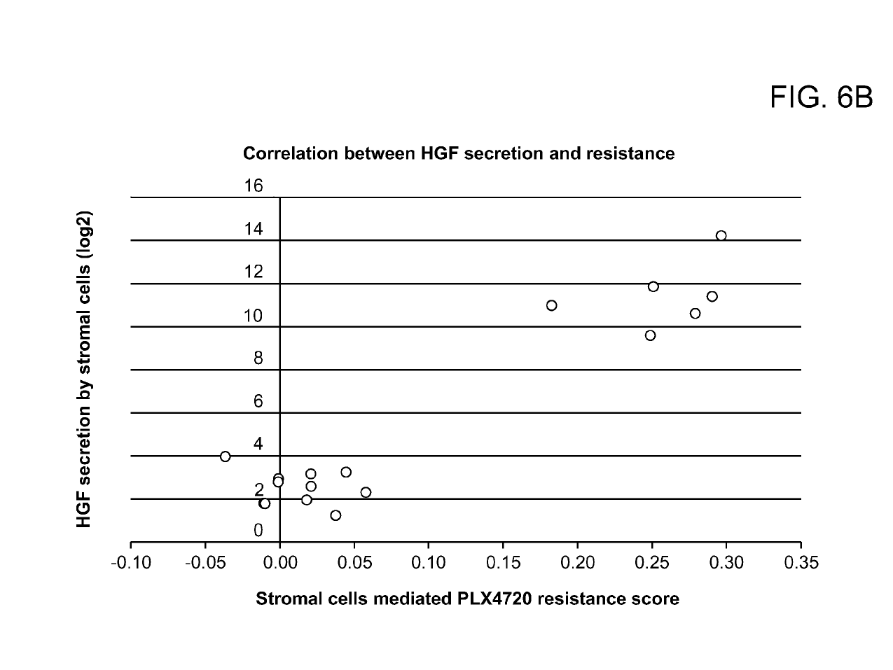

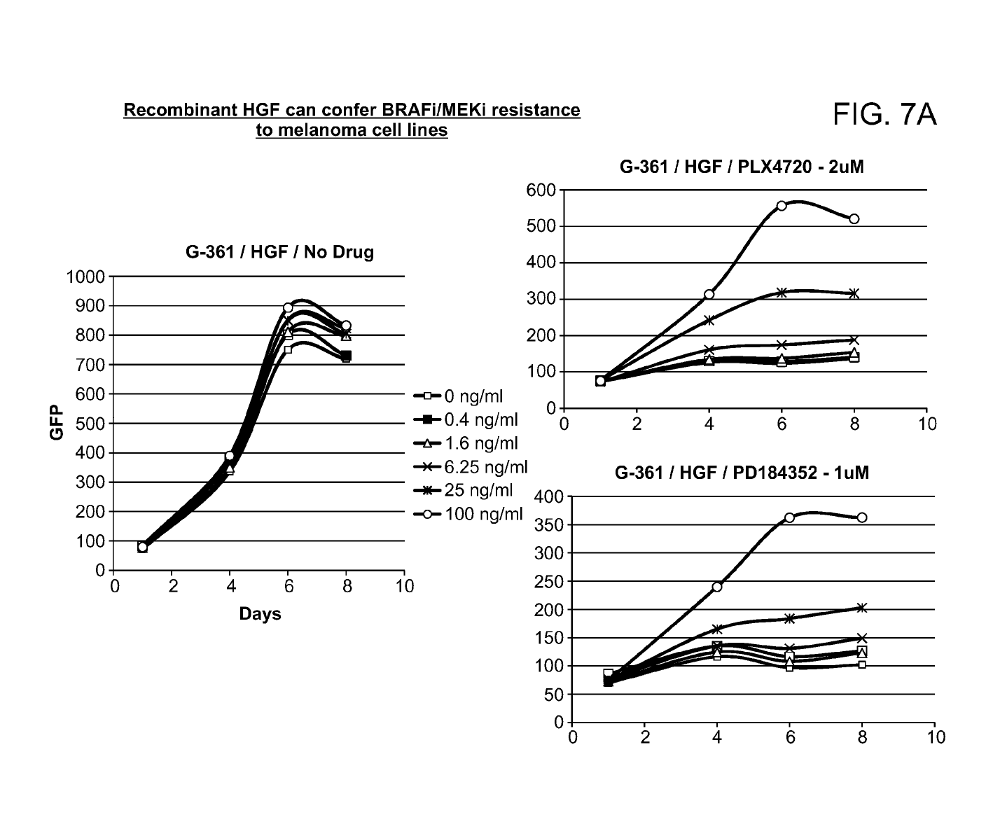

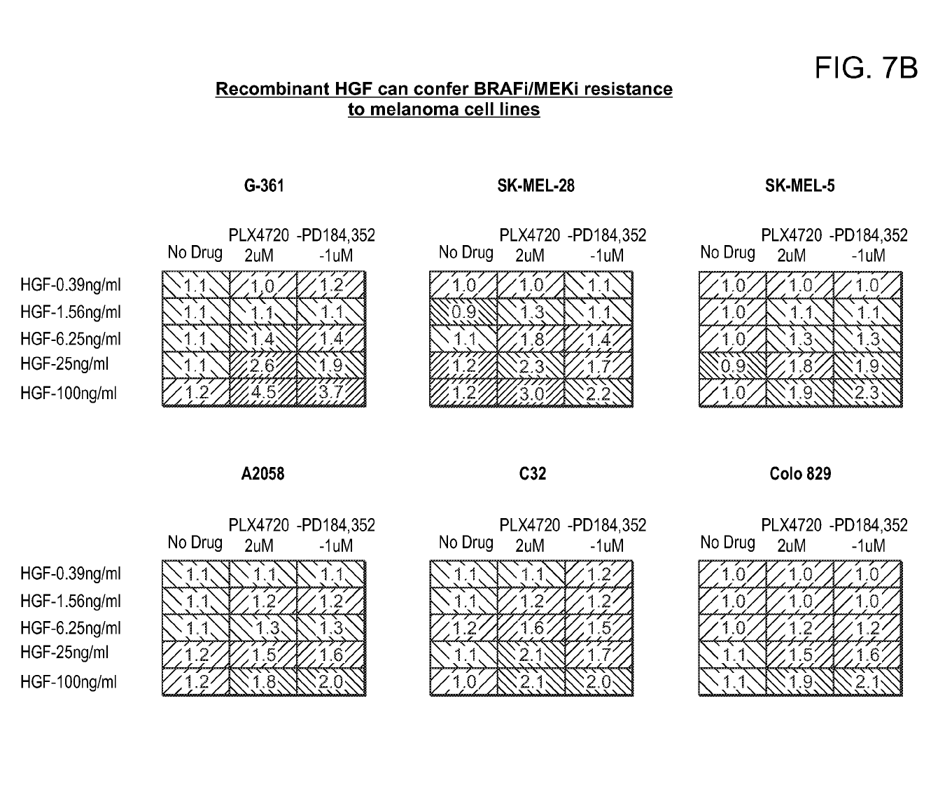

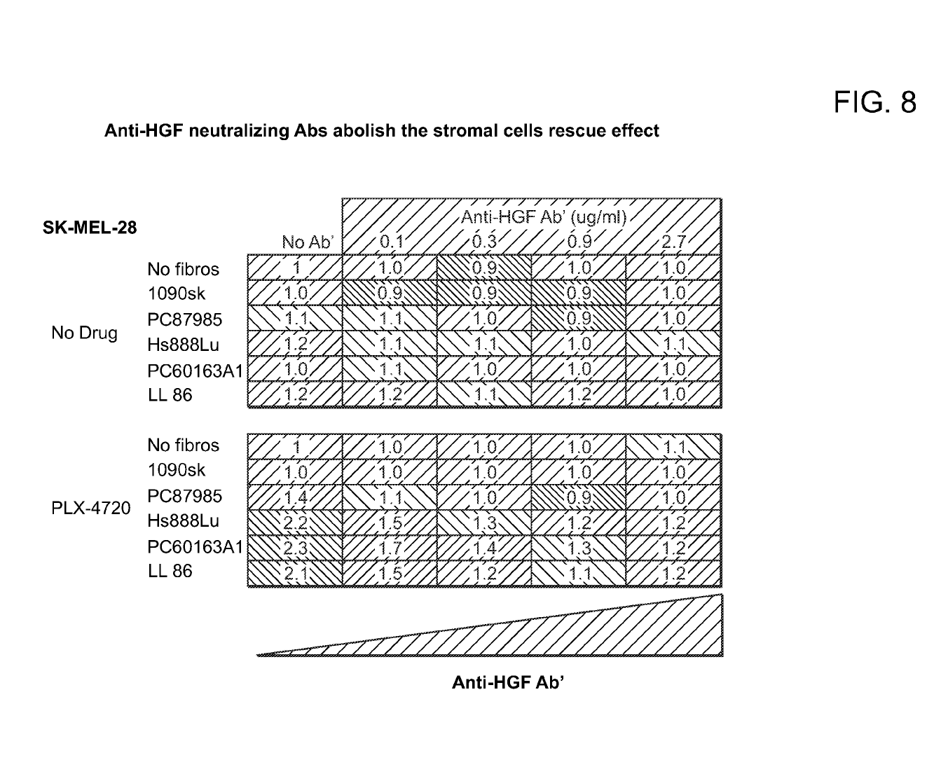

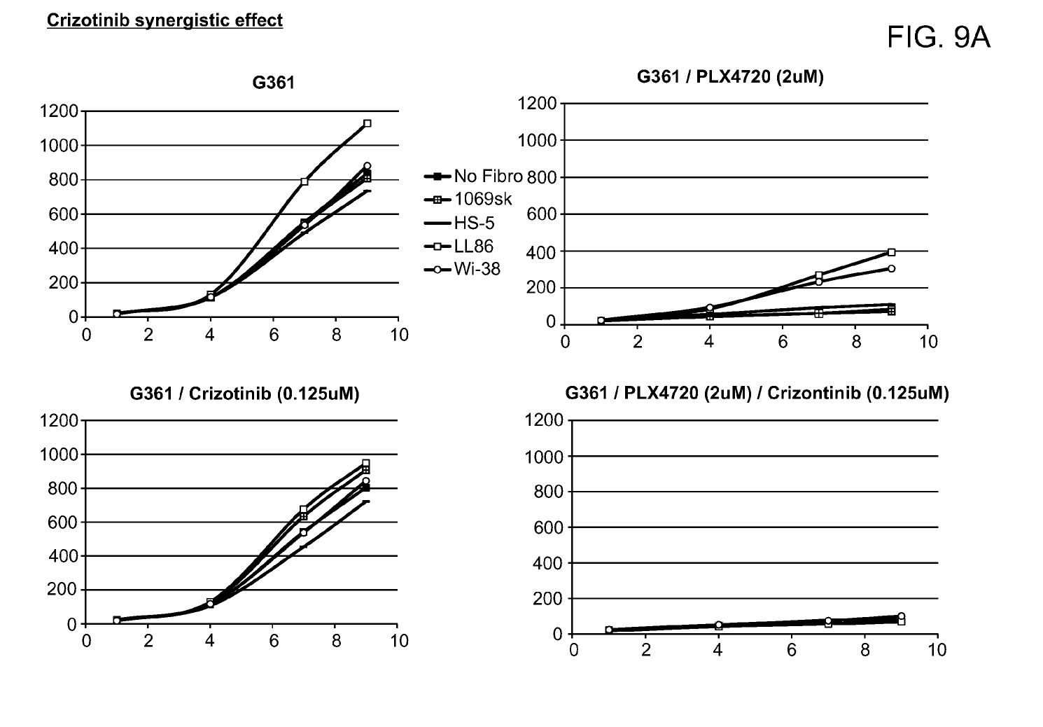

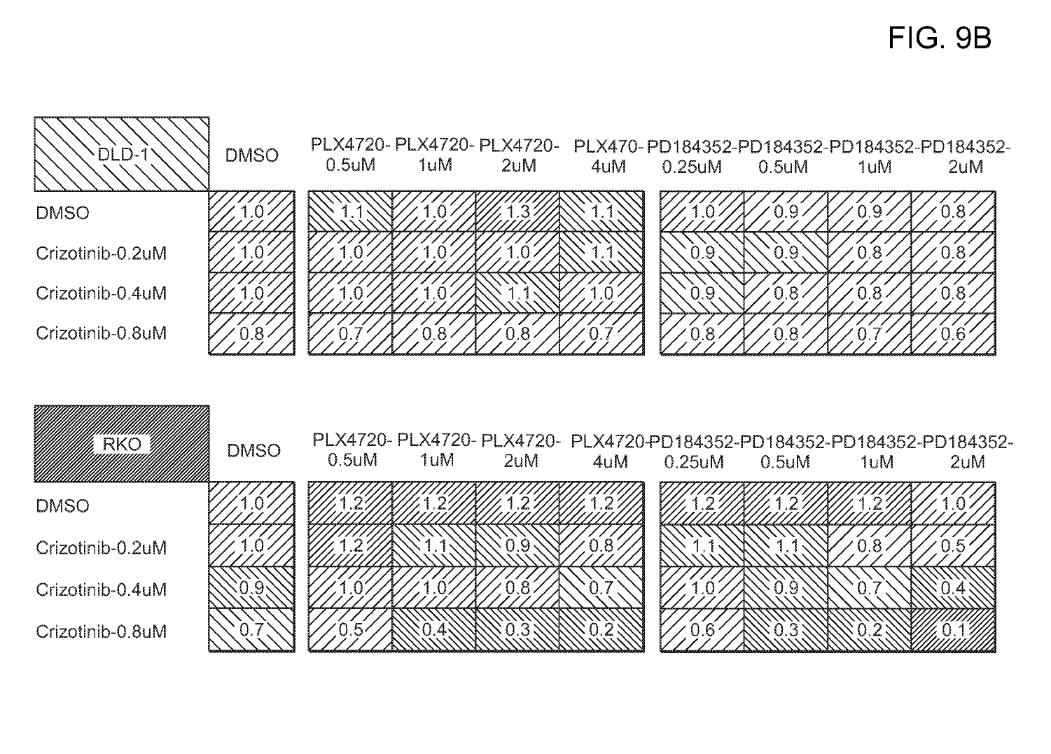

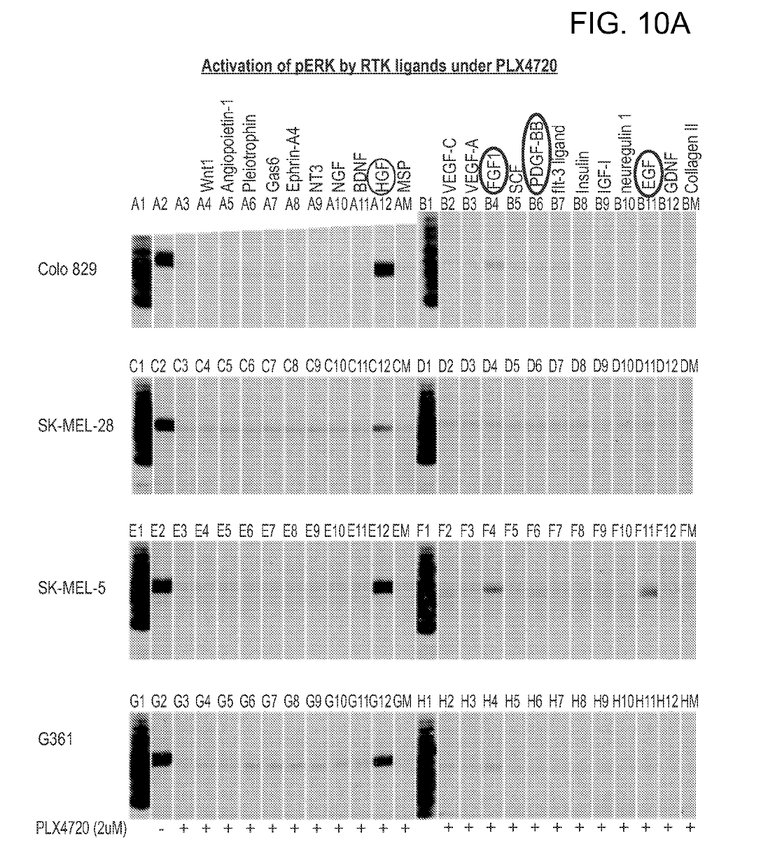

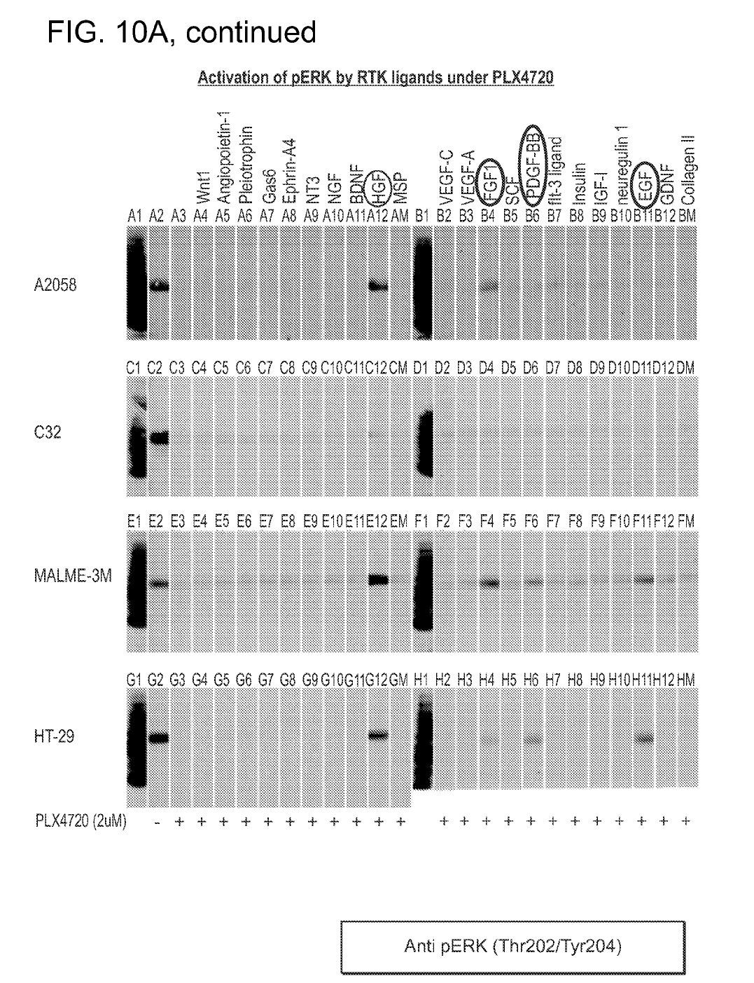

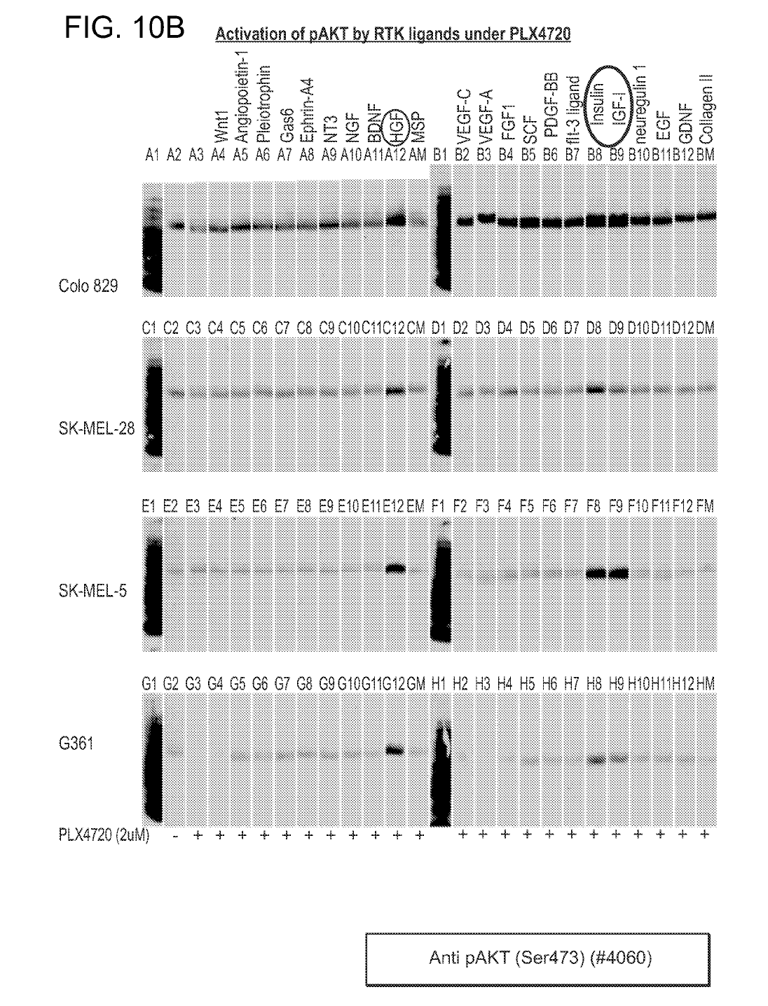

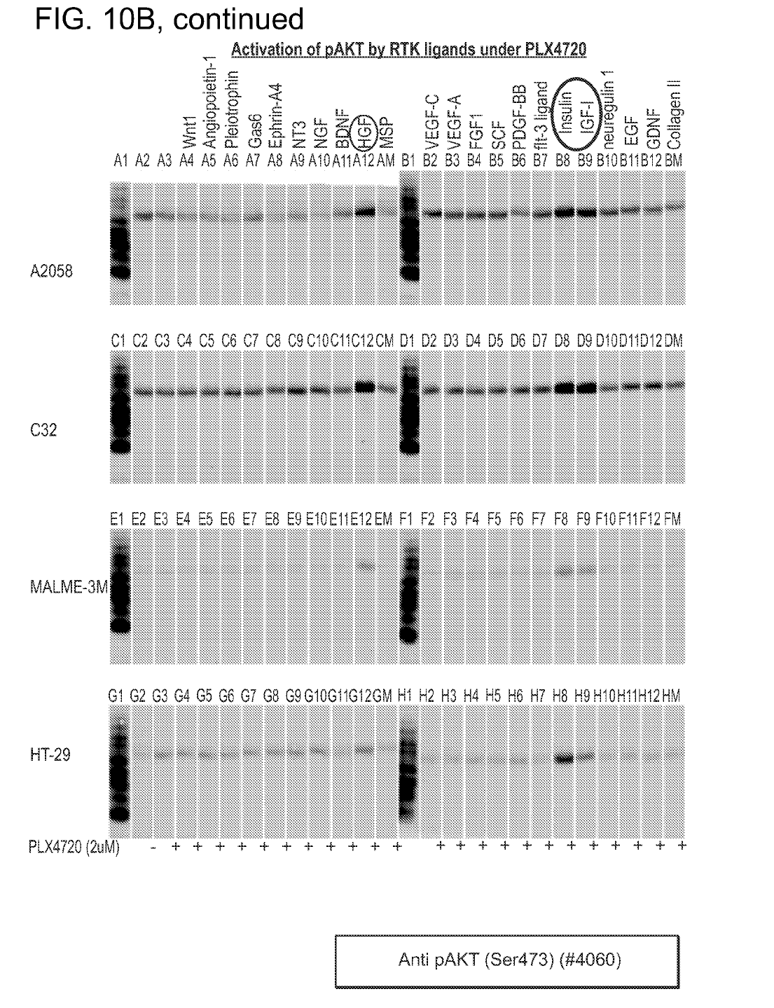

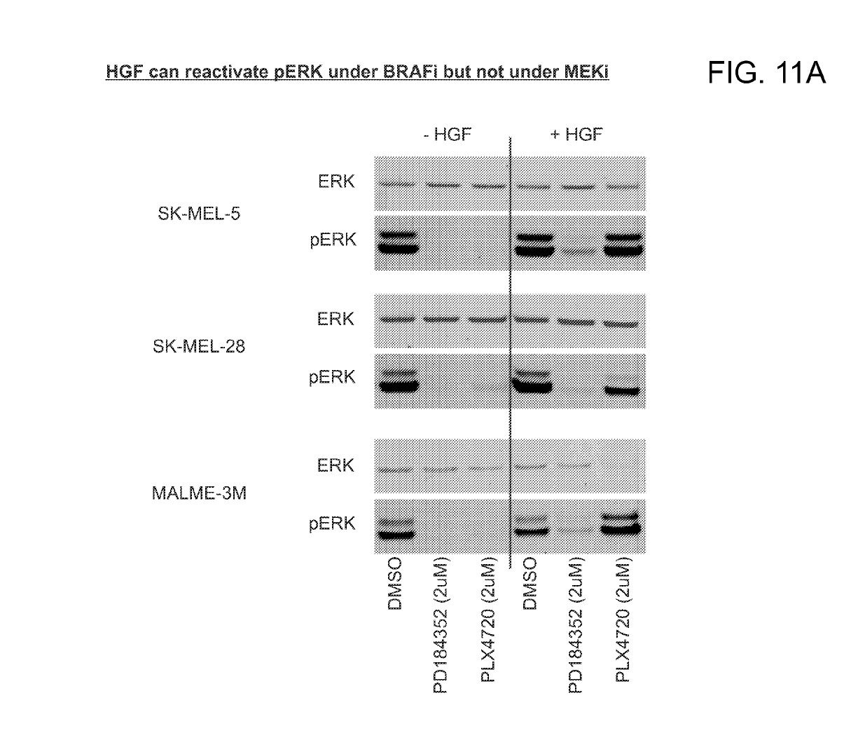

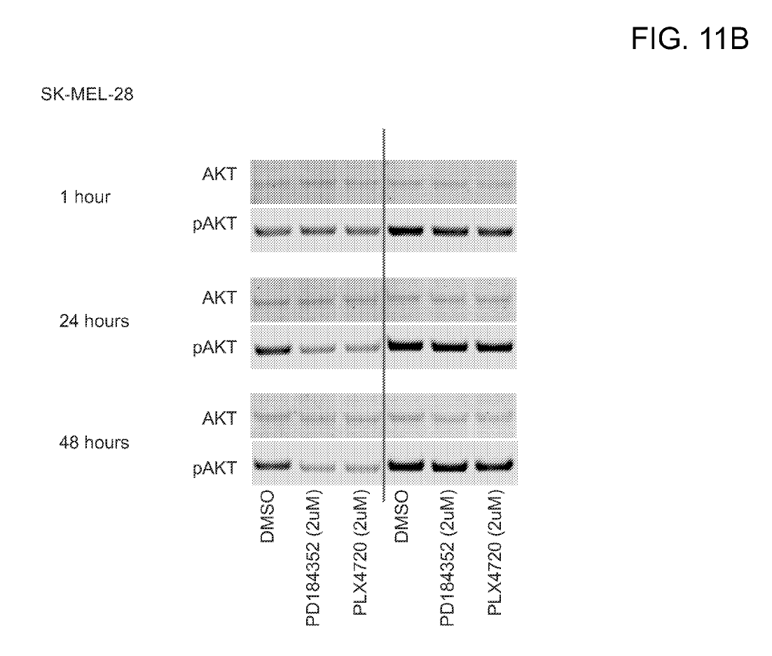

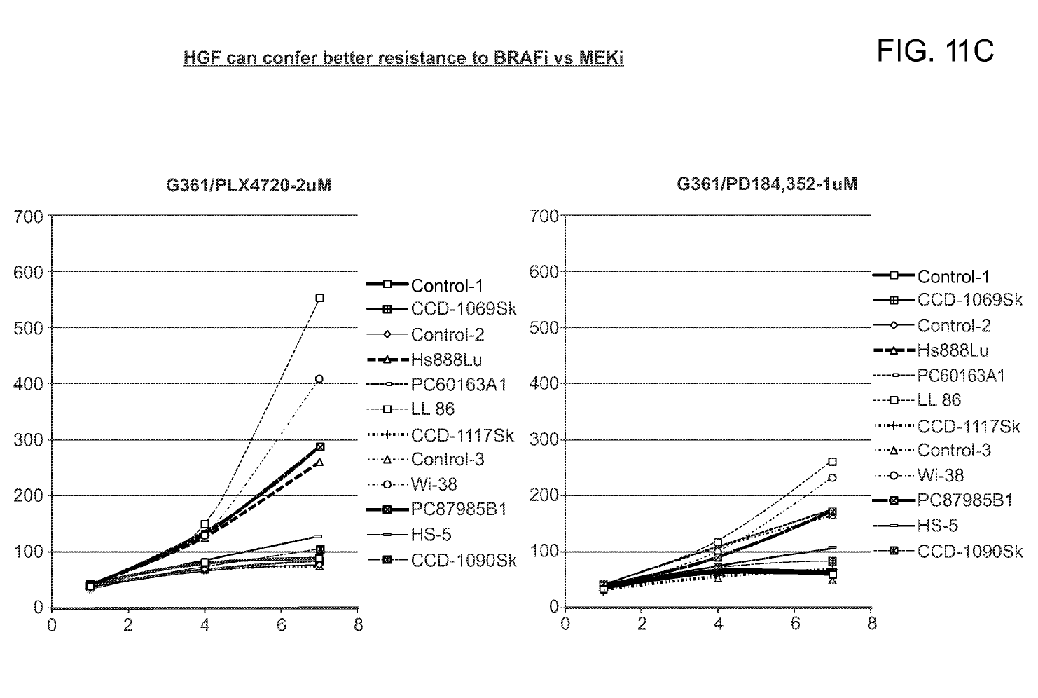

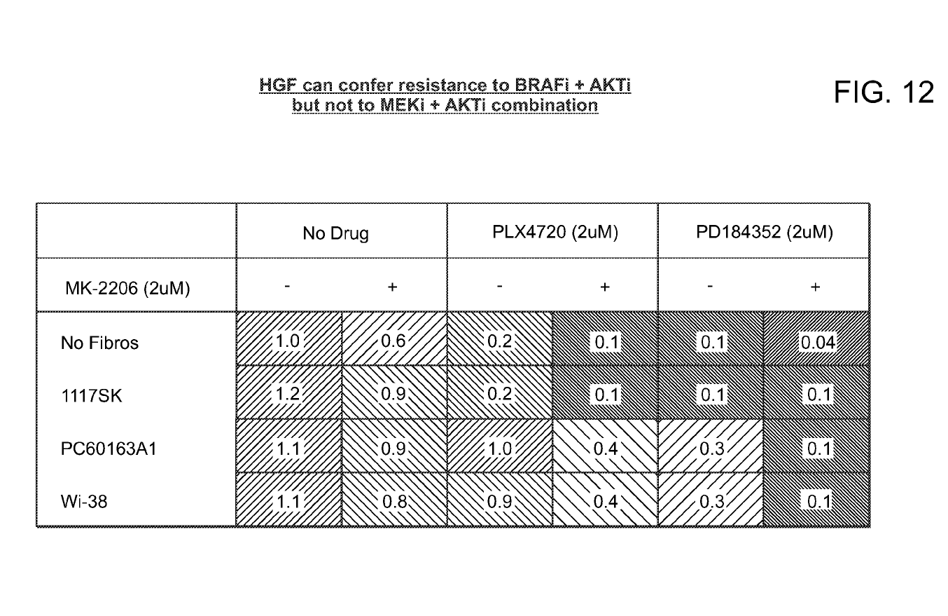

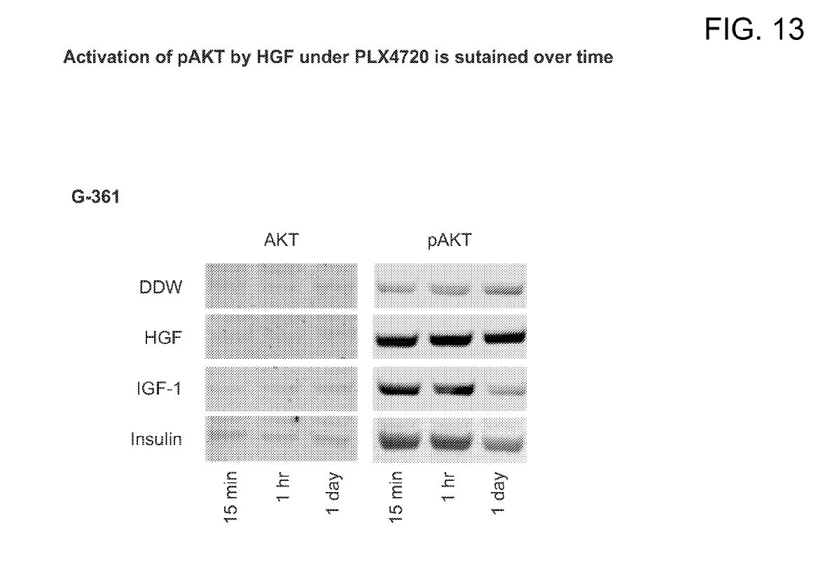

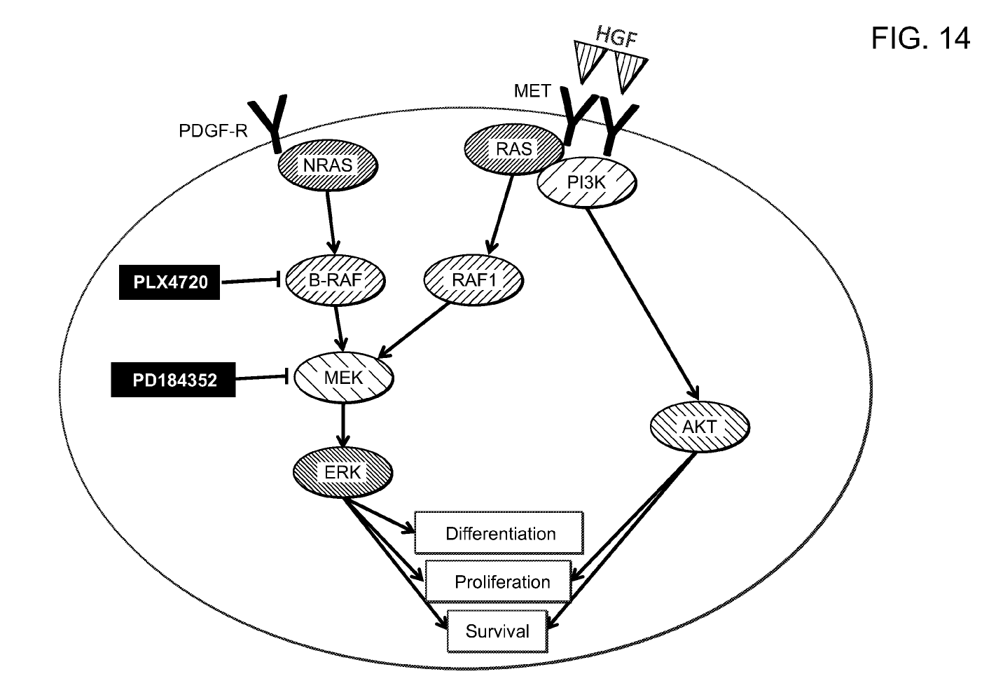

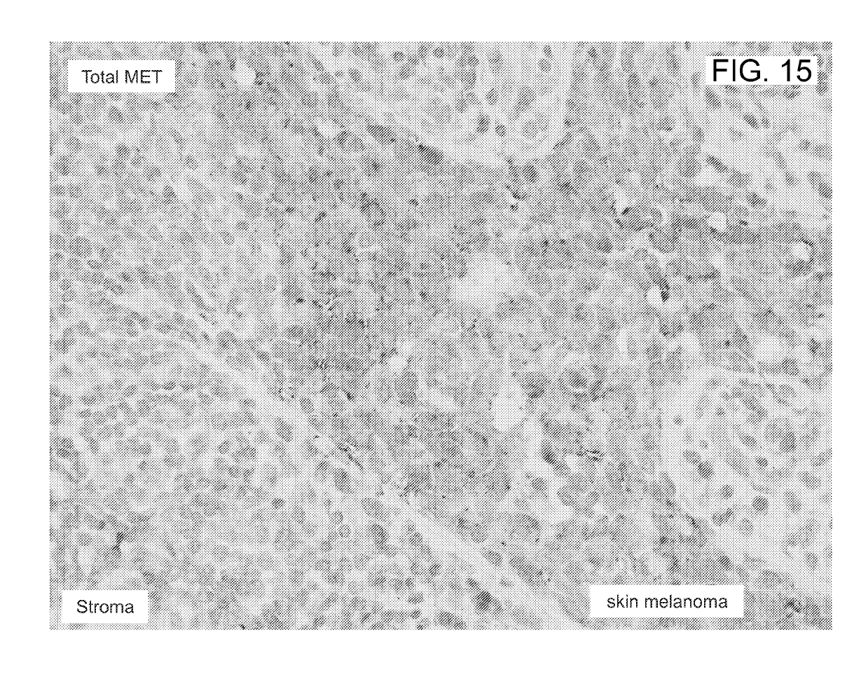

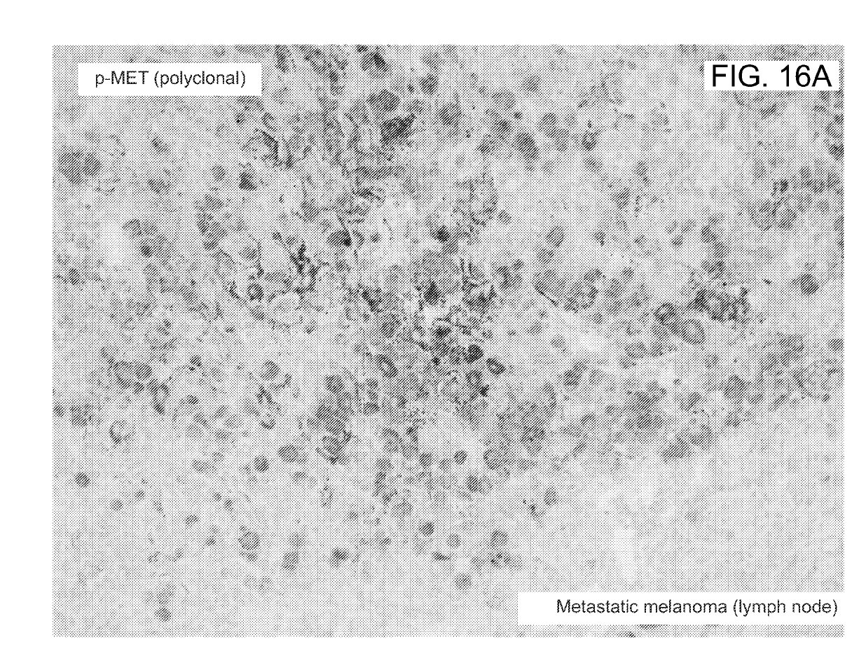

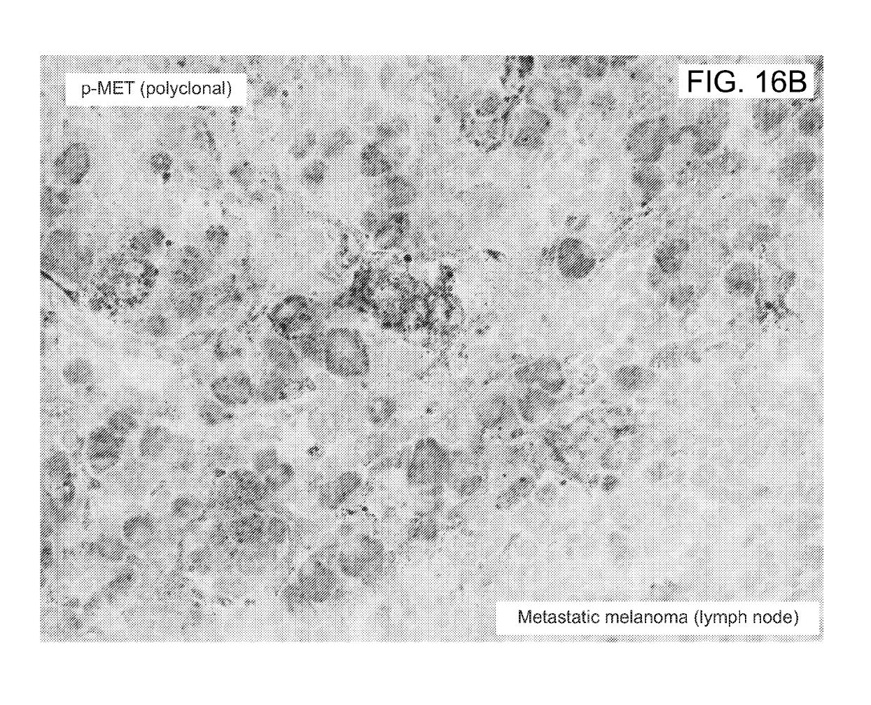

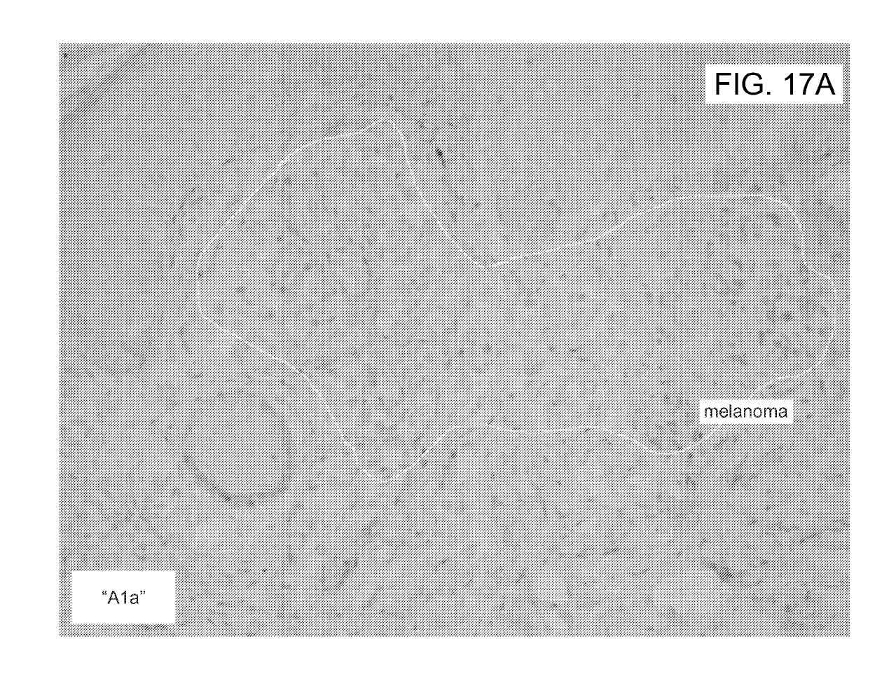

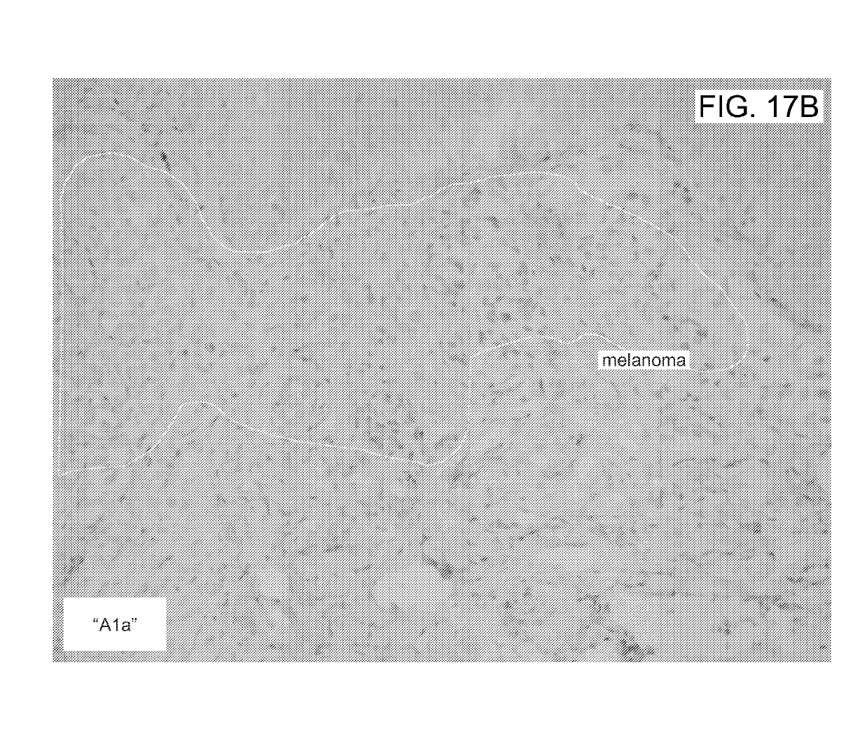

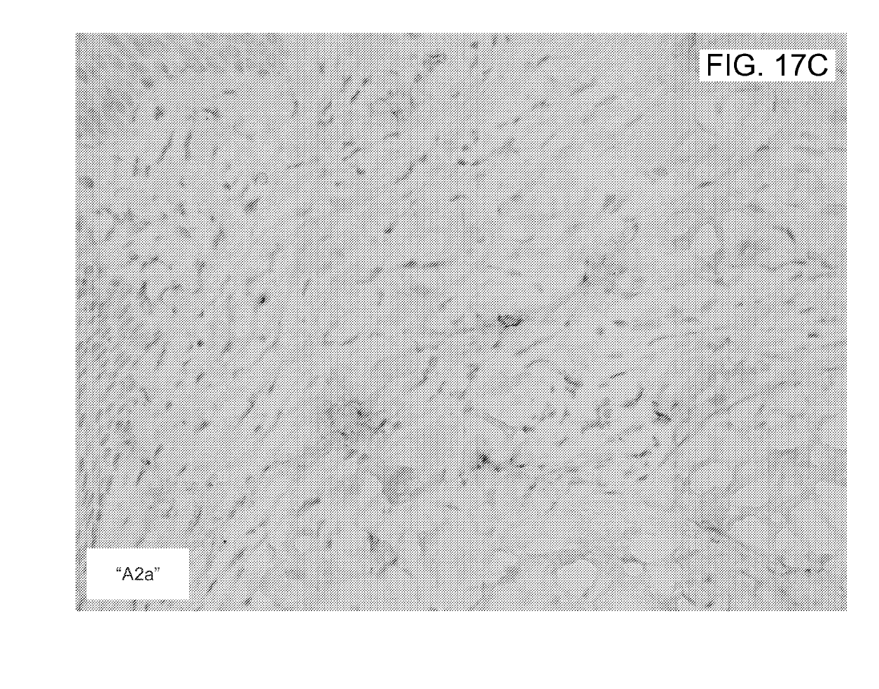

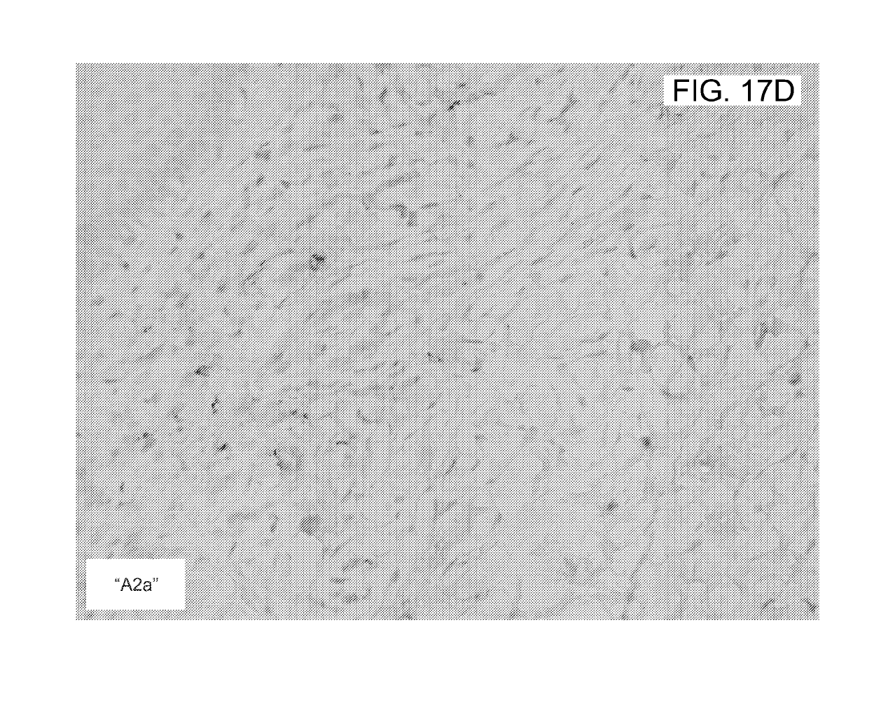

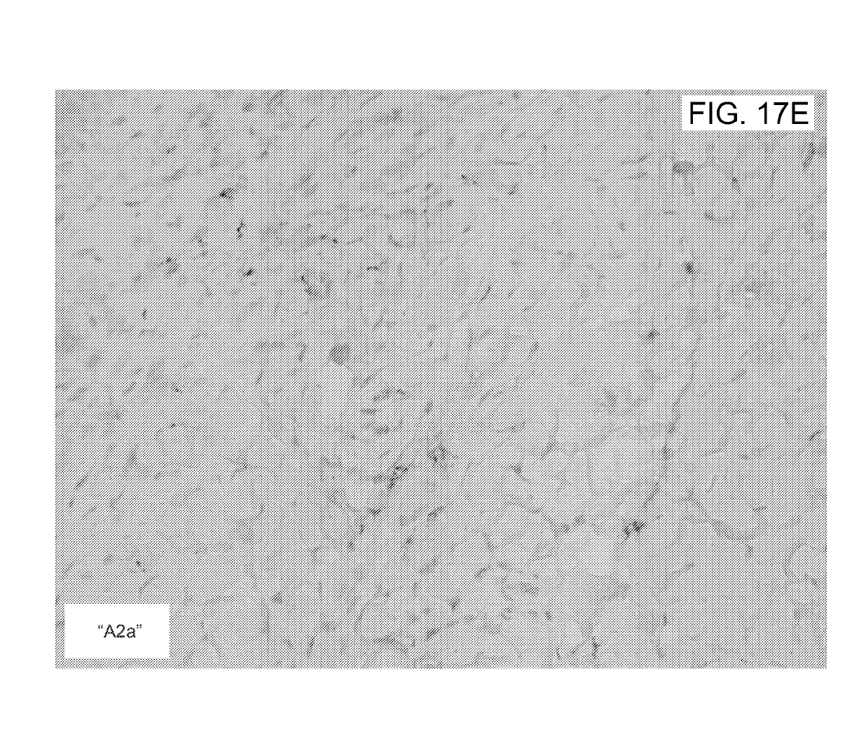

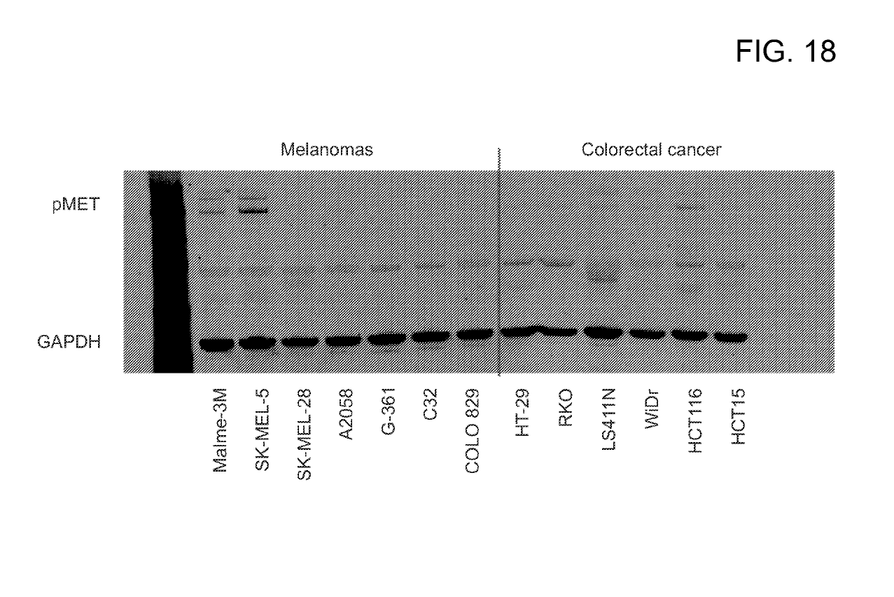

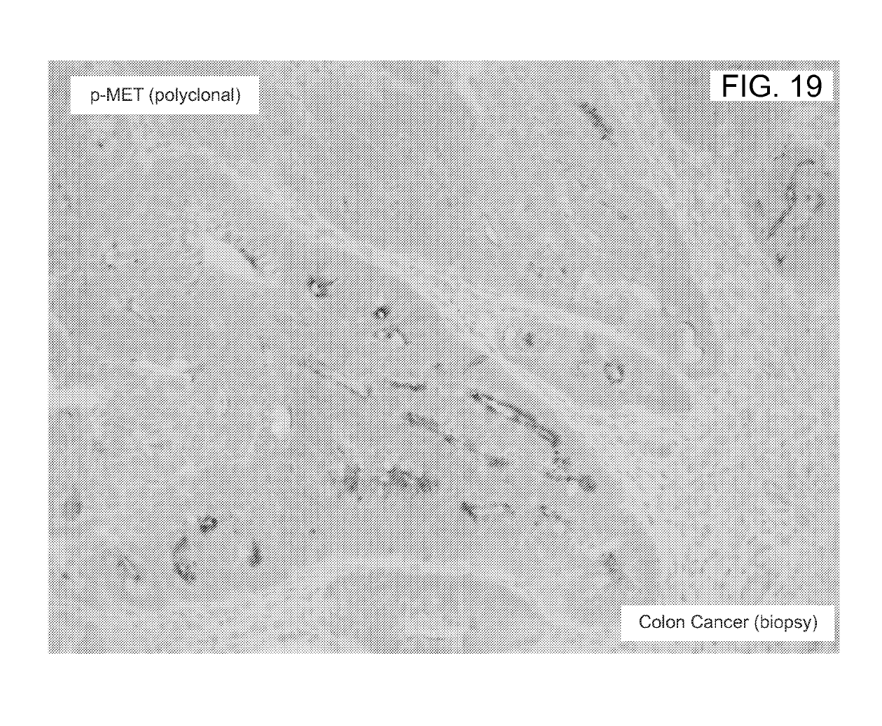

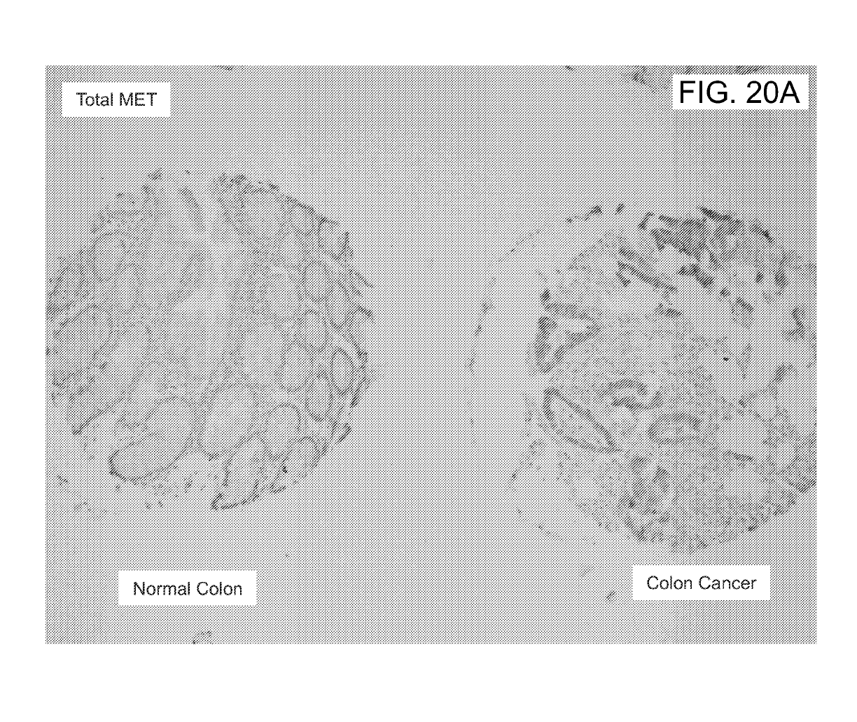

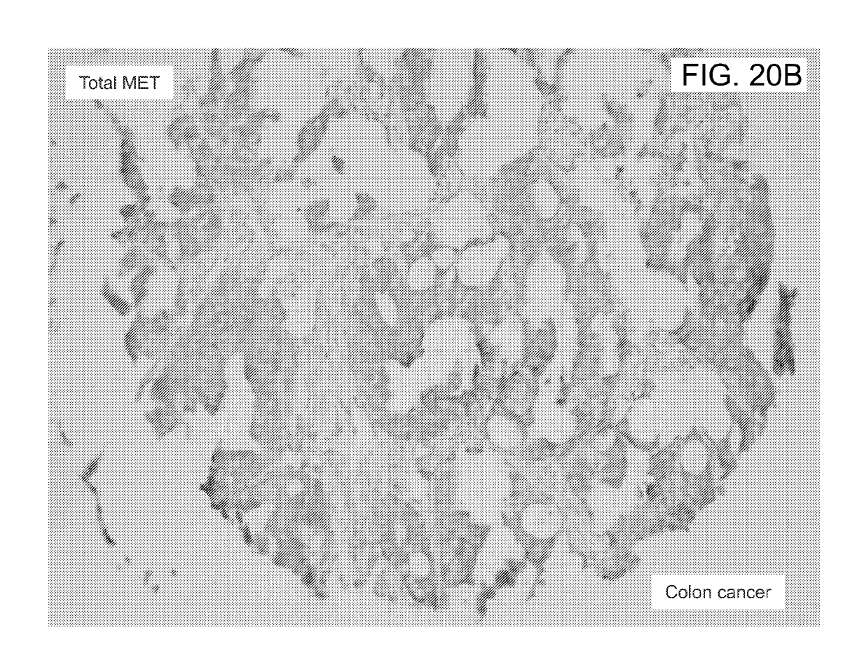

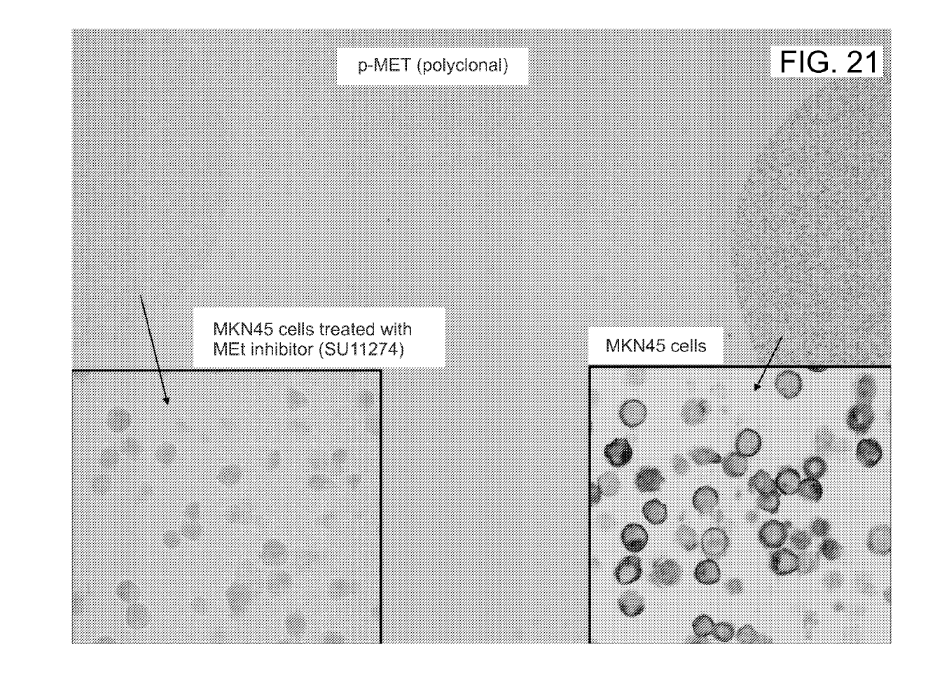

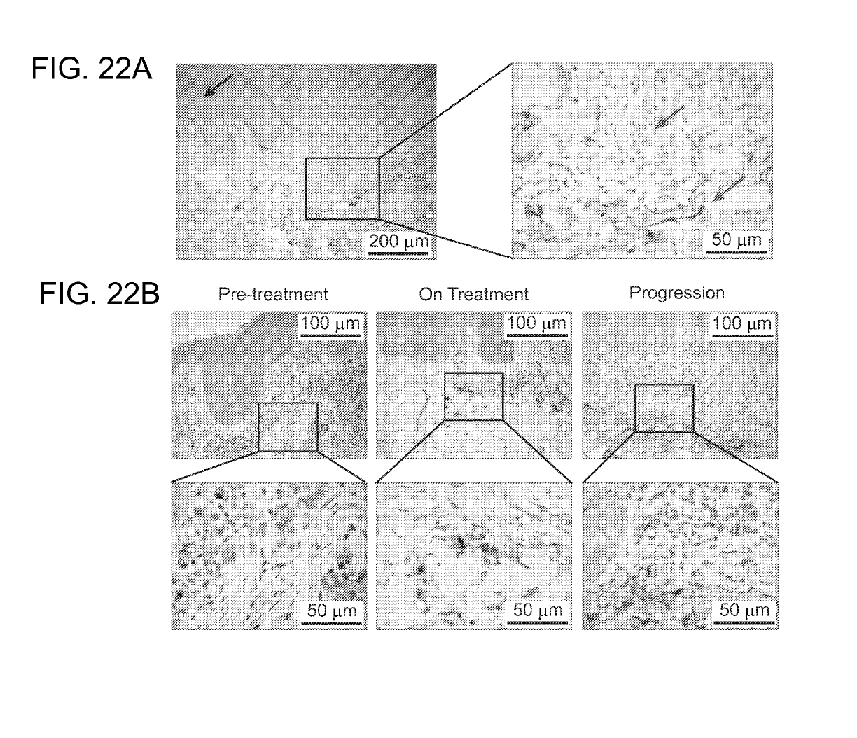

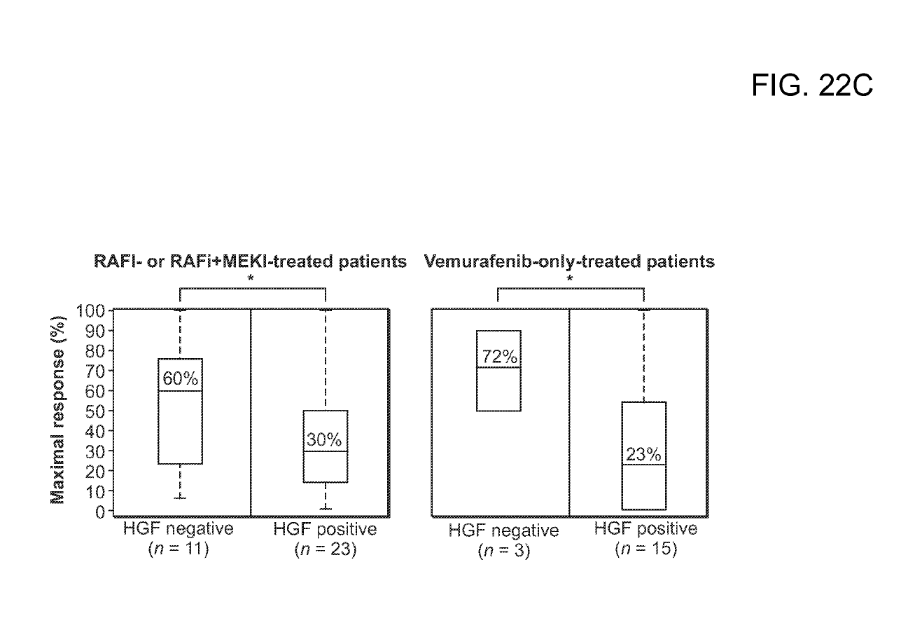

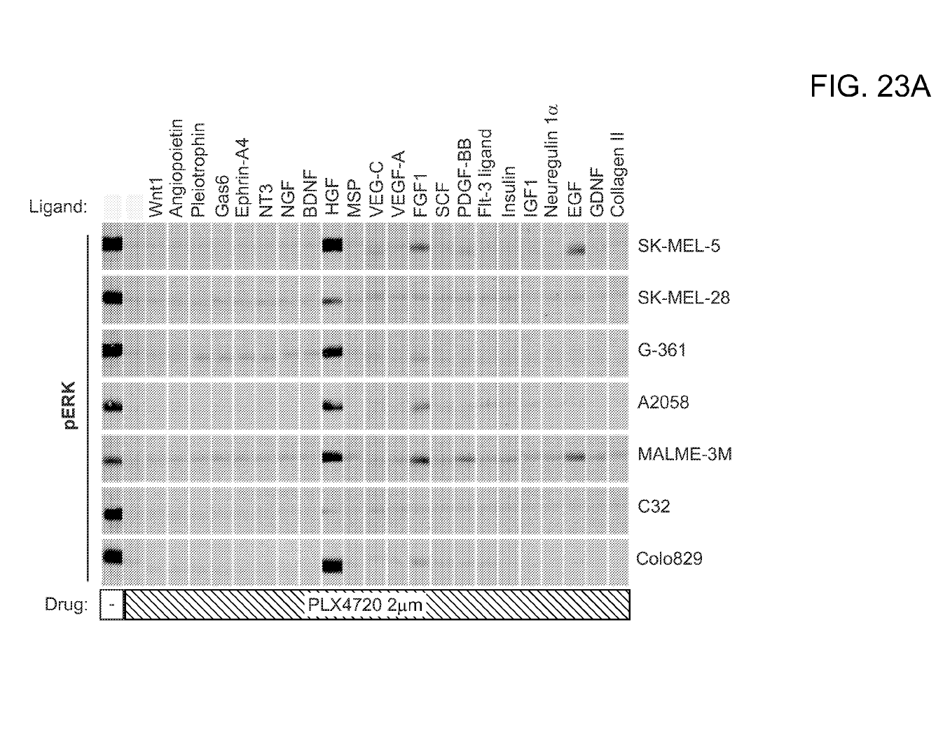

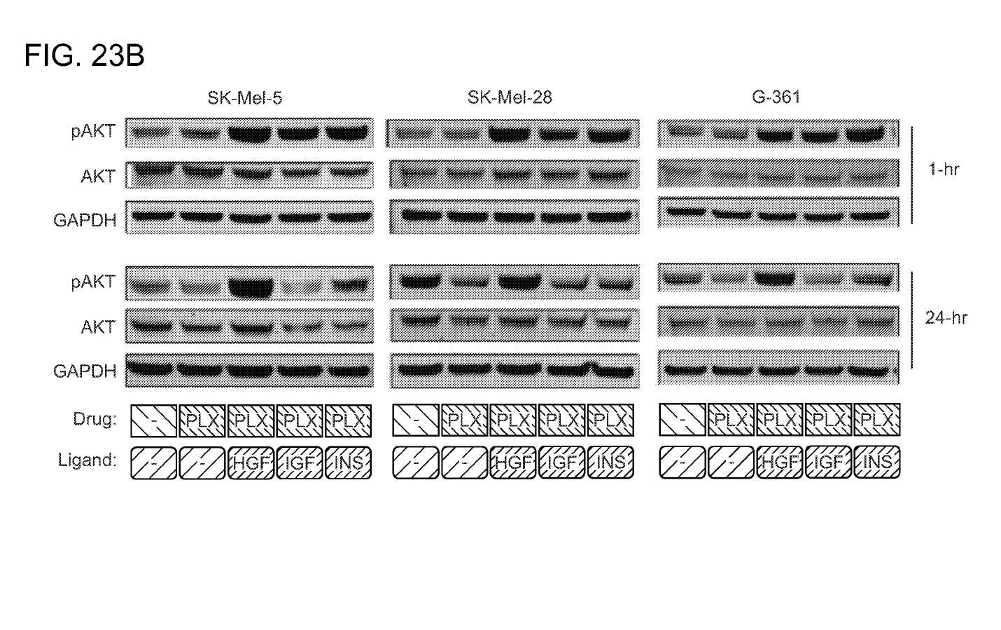

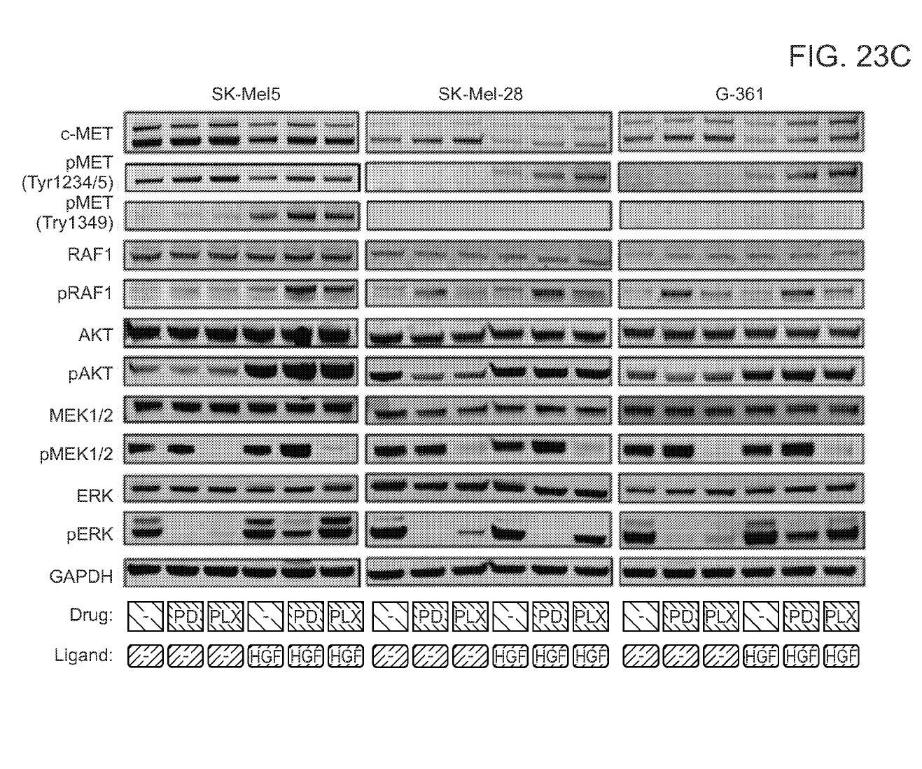

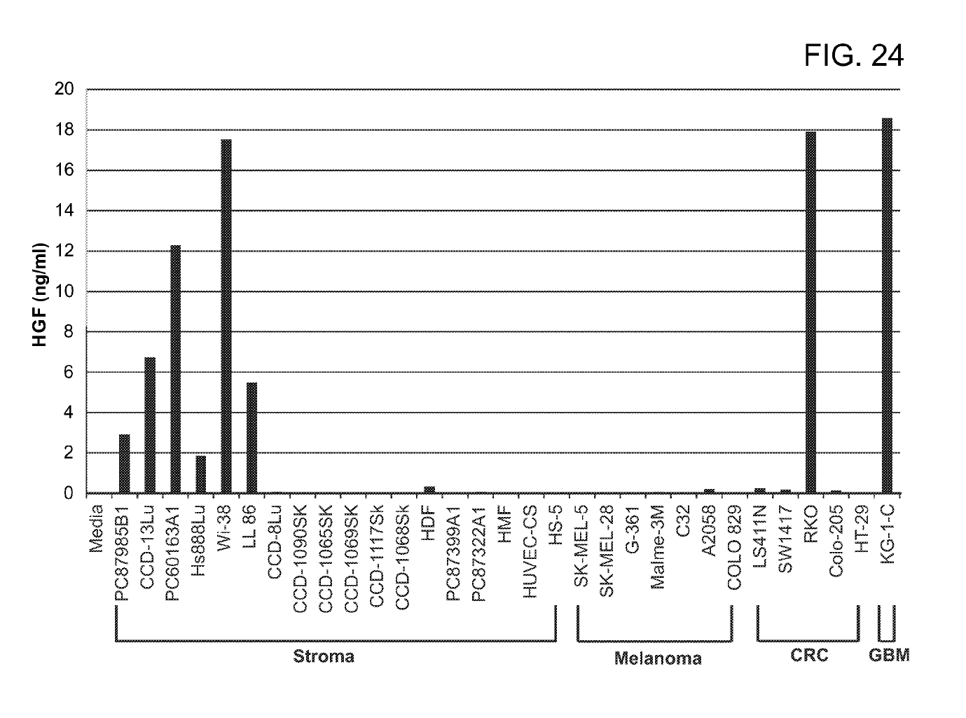

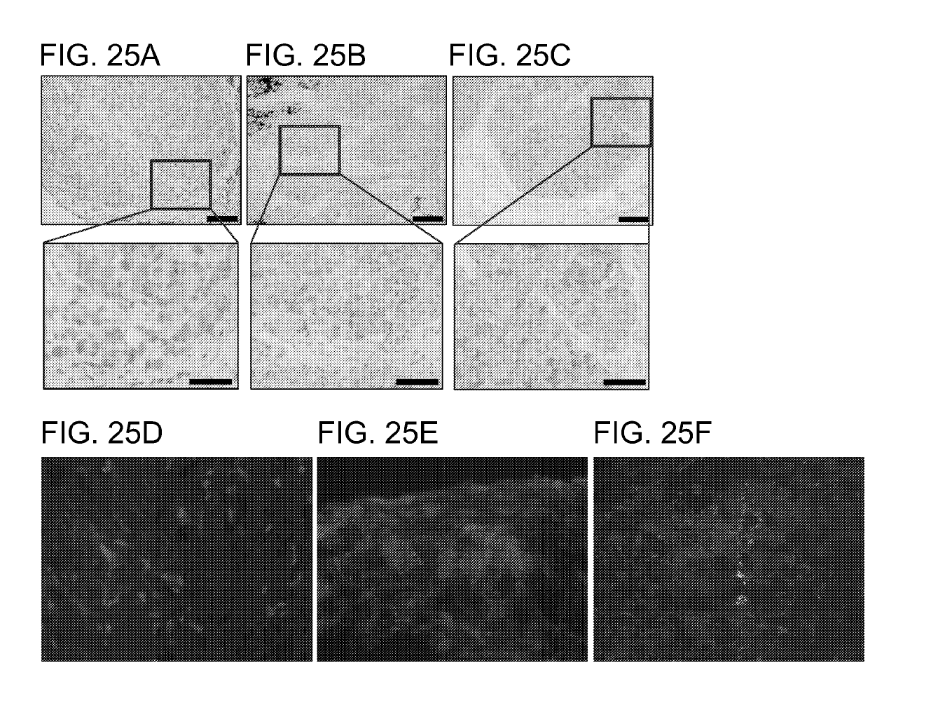

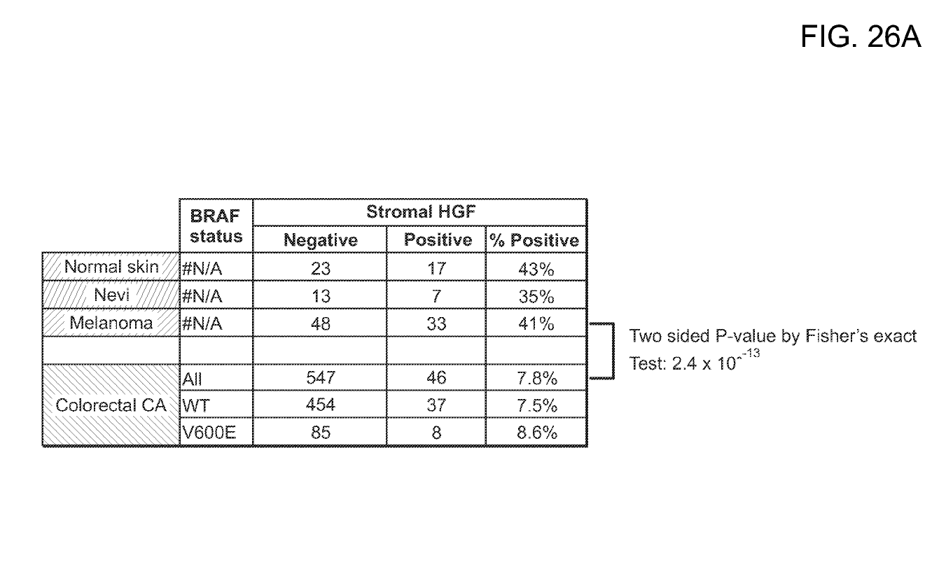

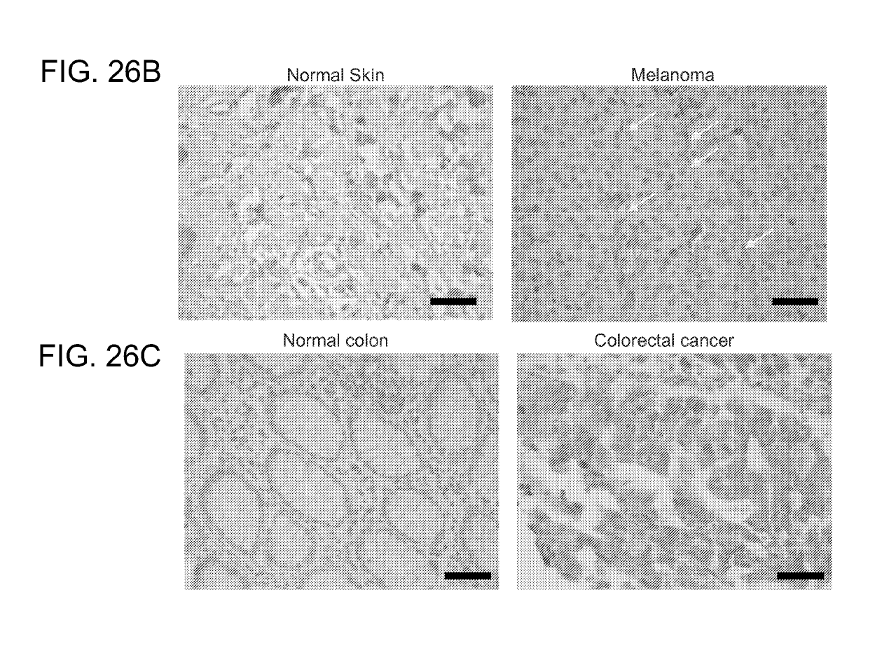

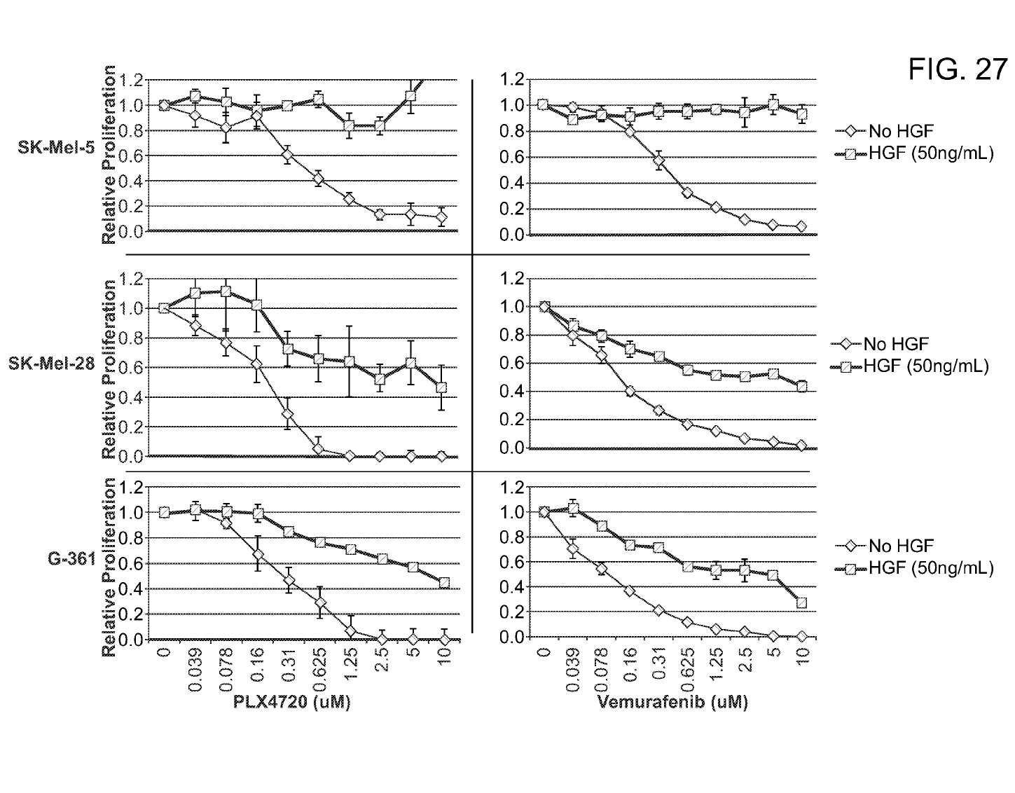

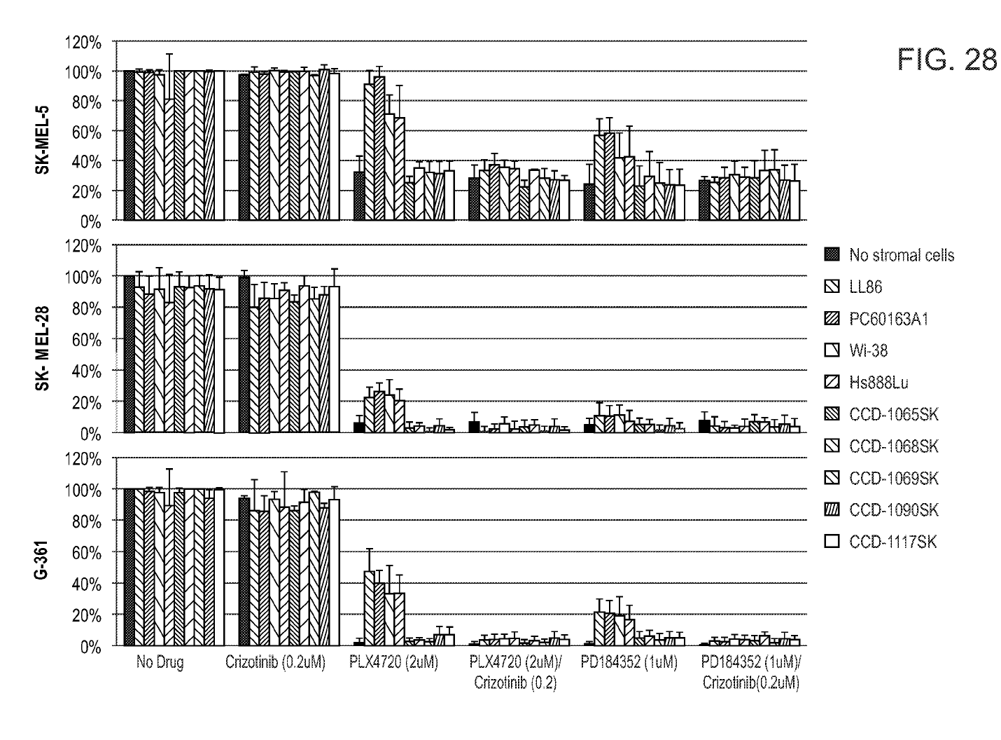

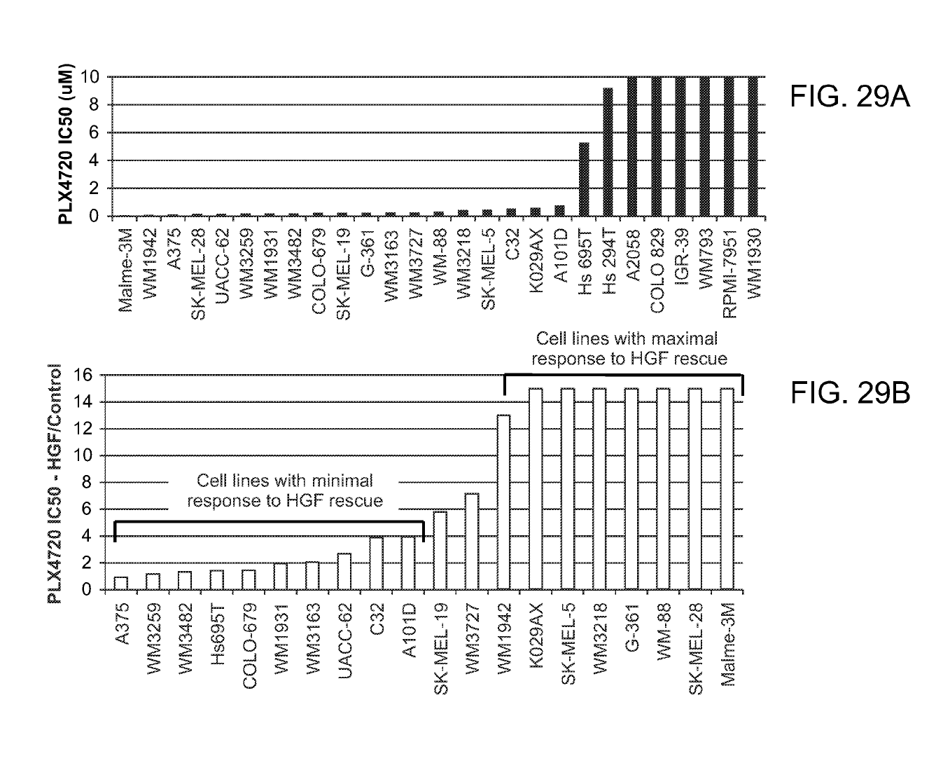

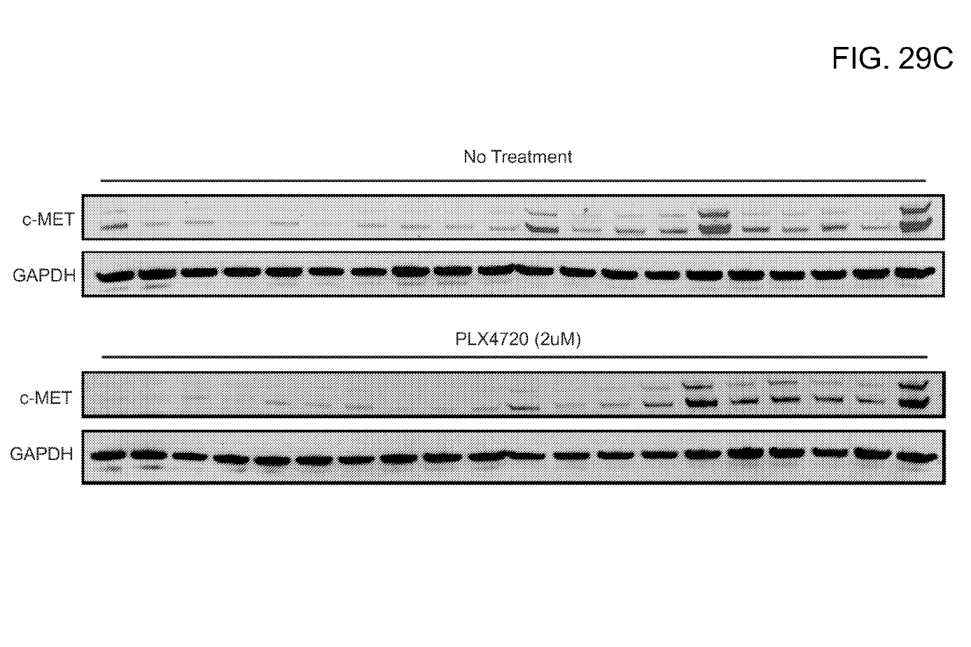

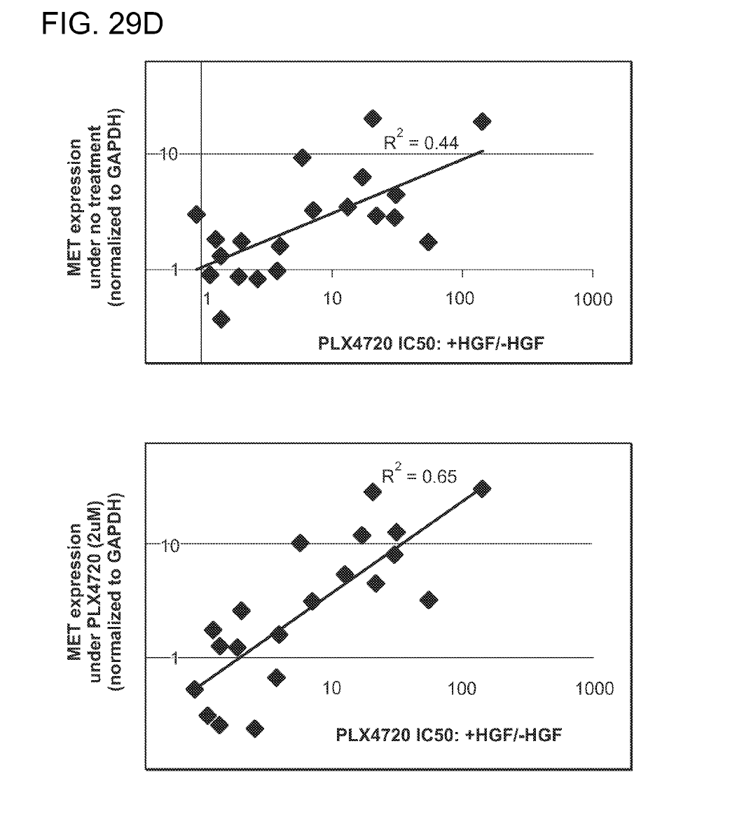

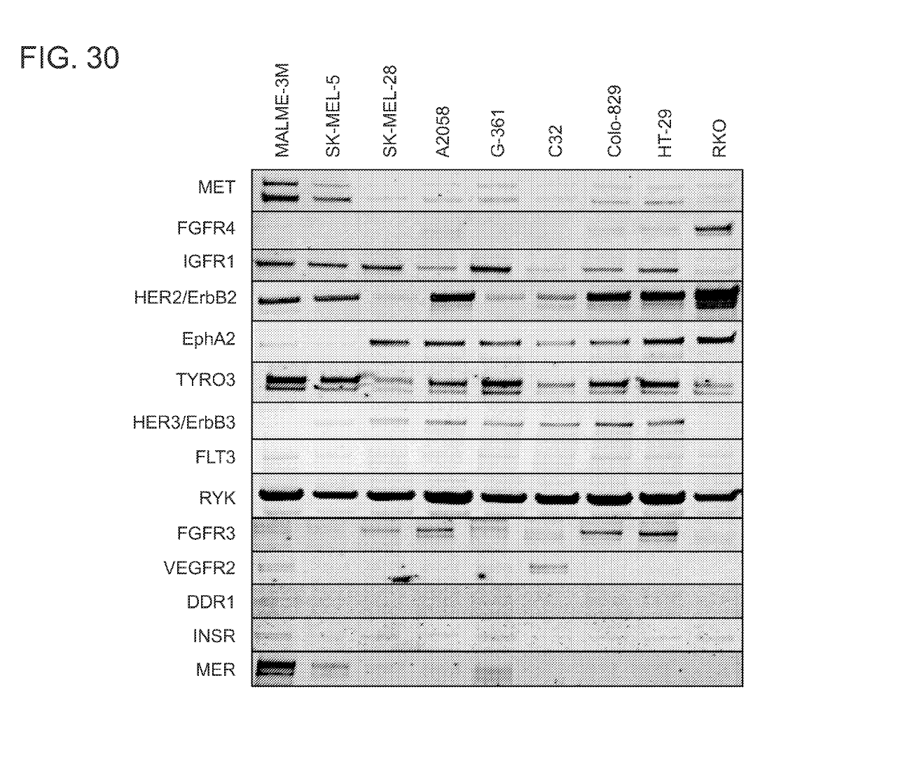

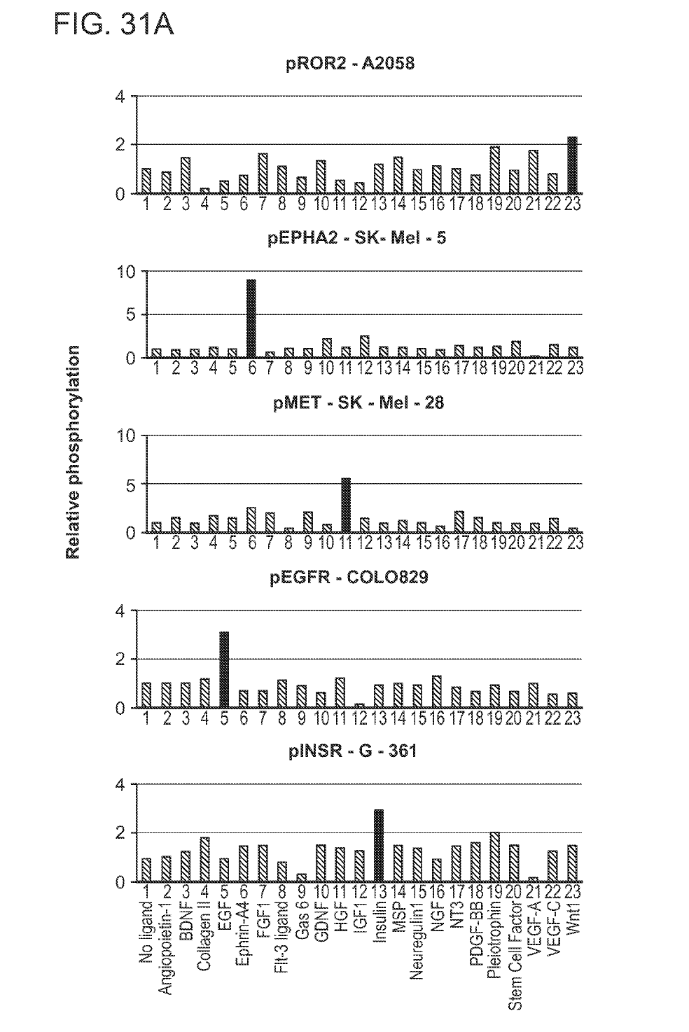

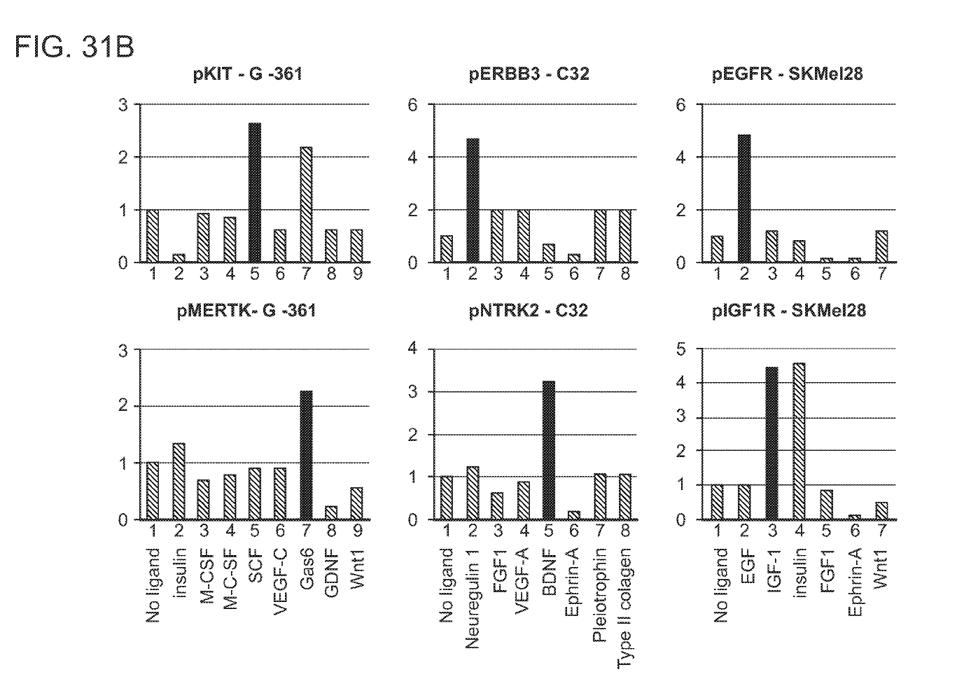

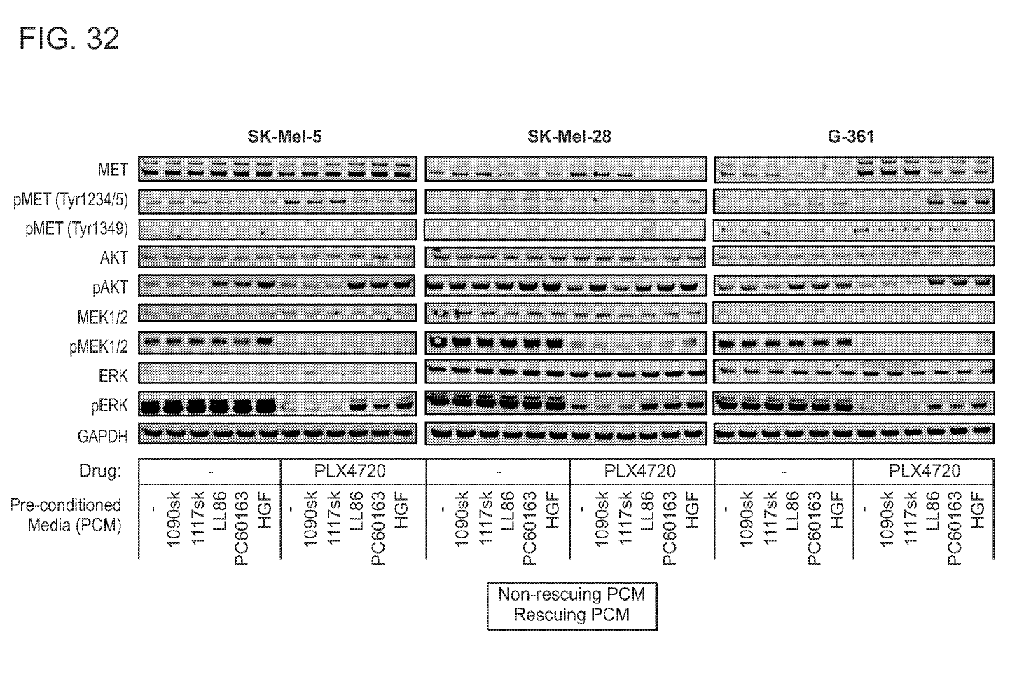

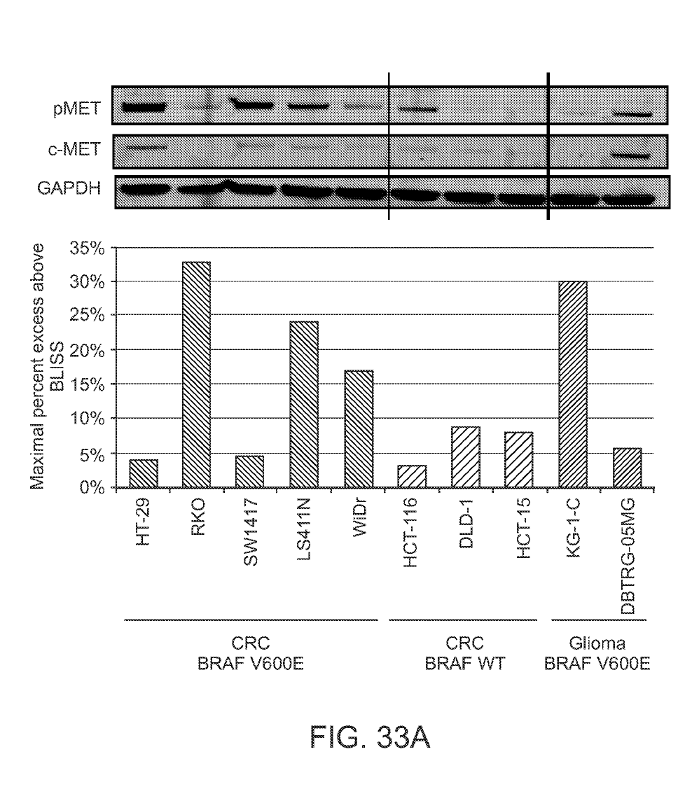

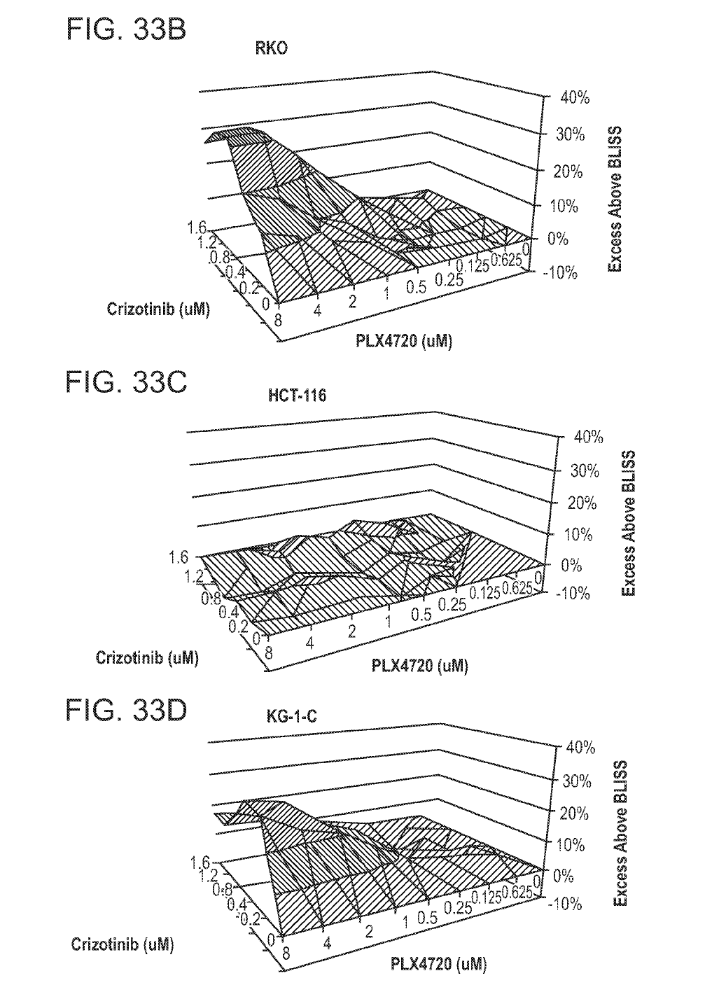

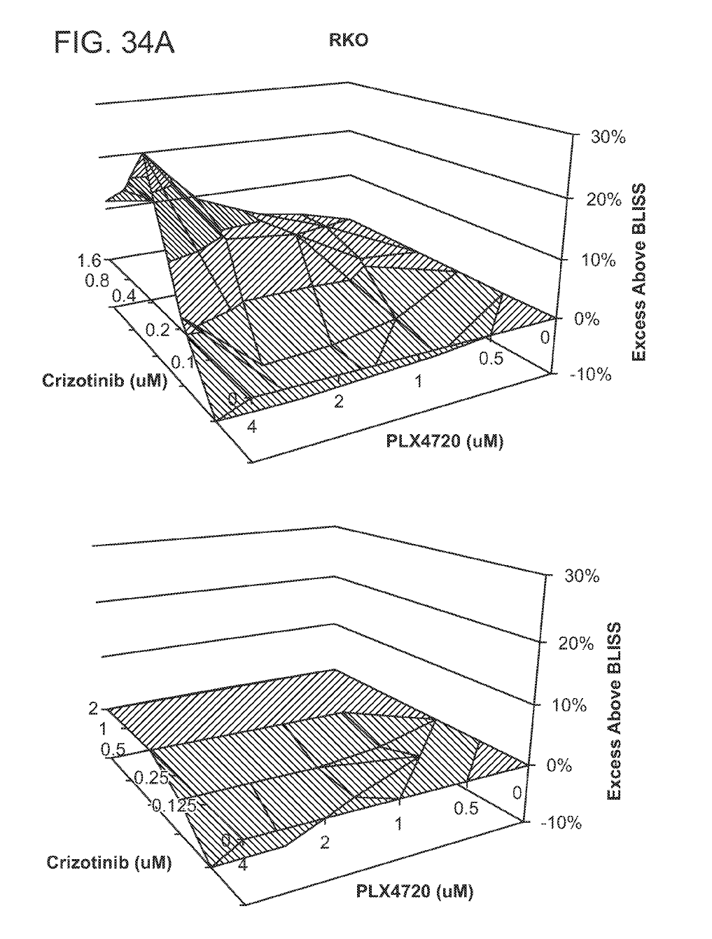

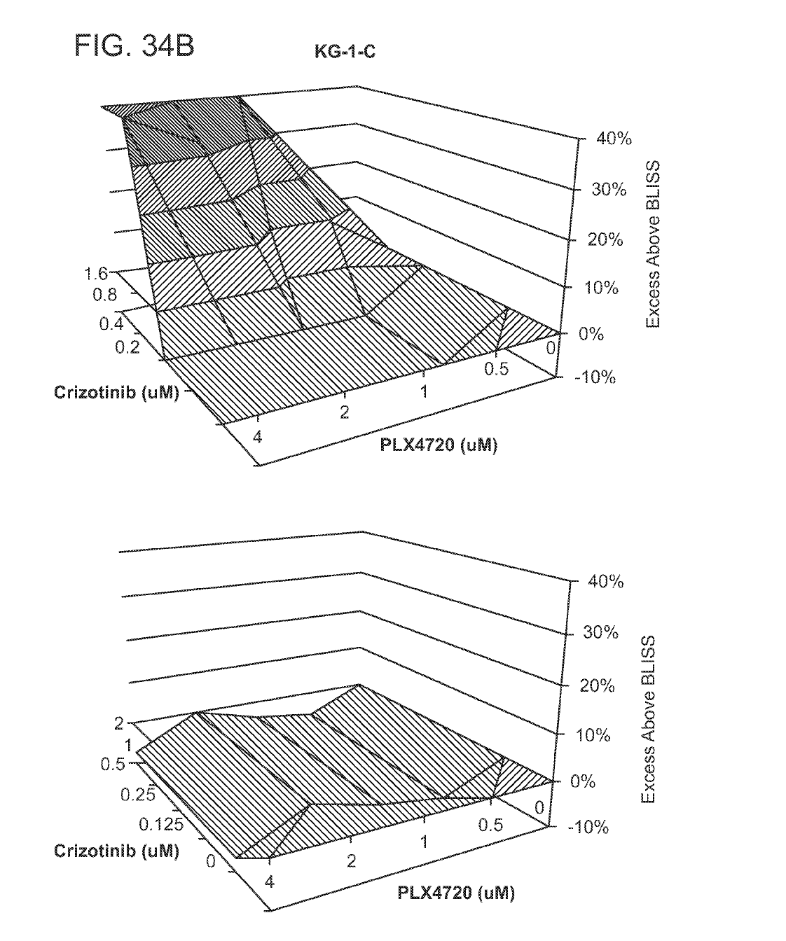

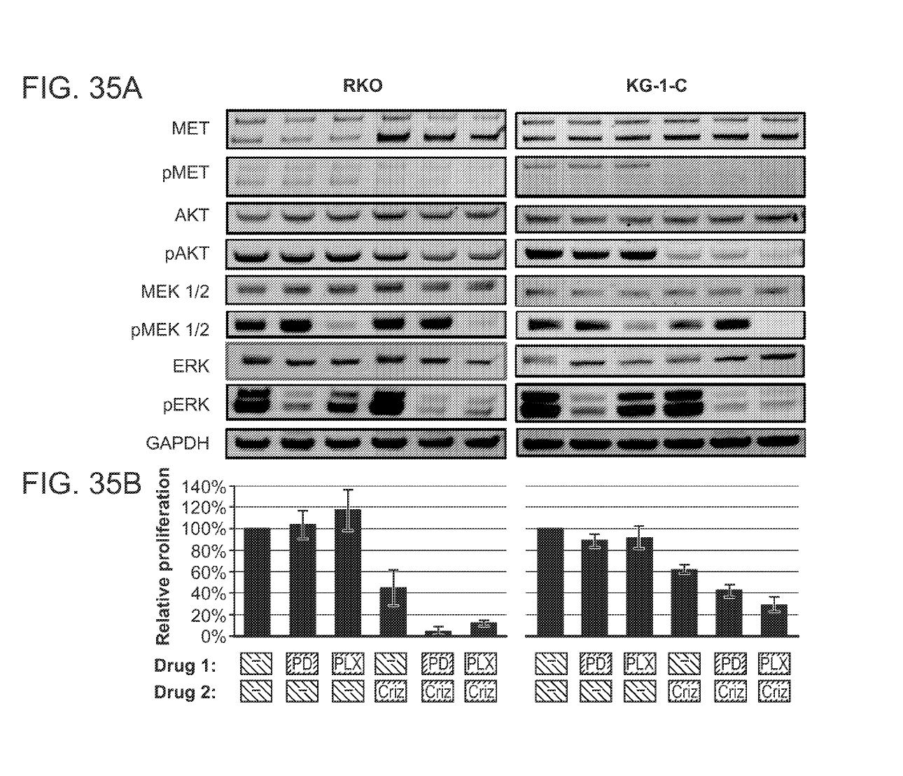

The propensity of tumors to develop resistance to a wide range of chemotherapy drugs imposes a critical hurdle to the treatment of most types of cancers. While the role of the cellular microenvironment in tumor progression and metastasis has been increasingly studied in recent years, its effects on drug resistance are still underappreciated and poorly understood. An optimized high-throughput screening system was used to explore the prevalence and significance of stromal cell-mediated chemoresistance in solid tumors. Forty-six GFP-labeled human cancer cell lines (Table 1) were cultured on 384-well plates either alone or co-cultured with each one of 23 human stromal cell lines (Table 3). The effects of a diverse range of widely used chemotherapy drugs (Table 2) on the proliferation of the single- or co-cultured cancer cell lines were assessed through GFP readings and through fluorescence microscopy. Stroma-mediated primary chemoresistance was clearly detected across most cancer cell lines and chemotherapy drugs (FIGS. 1A and 1B, Tables 3 and 4). In most cases, only a small subset of the stromal cells could render a specific cancer-cell line more resistant to a specific drug, and this subset was usually the same across cancer cell lines from the same origin (FIGS. 1-4). In some cases, the subset of stromal cell lines that induced drug resistance was preferentially derived from the same tissue of origin as the affected cancer cell lines. It was also evident that stromal cell lines had a more substantial effect on targeted drugs than on classic cytotoxic agents (FIG. 1B).

A large subset of patients with BRAF-mutant cancers exhibits some degree of innate drug resistance. Characterization of the stroma-mediated resistance of BRAF-mutant melanoma to RAF inhibition is described herein. Proteomic analyses showed that stromal secretion of the hepatocyte growth factor (HGF) resulted in activation of the HGF receptor MET, reactivation of the MAPK and PI3K/AKT pathways, and immediate resistance to RAF inhibition. Immunohistochemistry confirmed stromal HGF expression in patients with BRAF-mutant melanoma and a statistically significant correlation between stromal HGF expression and innate resistance to treatment. Dual inhibition of RAF and MET resulted in reversal of drug resistance of BRAF-mutated melanoma cell lines. These findings have immediate clinical implications as it prompts the addition of MET inhibitors to v-raf murine sarcoma viral oncogene homolog B1 (BRAF) inhibitors or mitogen activated protein kinase kinase (MEK) inhibitors.

More broadly, the present invention highlights the currently underappreciated importance of the tumor cellular microenvironment in directly mediating substantial primary chemoresistance in solid tumors.

Accordingly, the invention provides methods of preventing or reducing stromal cell mediated chemoresistance in a tumor by administering to a cancer patient a chemotherapeutic agent and a c-MET kinase (MET) inhibitor. Also included are methods of treating cancer by identifying in a tumor sample from a subject a BRAF activating mutation and administering a MET inhibitor and a chemotherapeutic agent.

Chemotherapeutic agents include, for example, RAF inhibitors (e.g. Vemurafenib or Dabrafenib), MEK inhibitors, PI3K inhibitors, or AKT inhibitors. The RAF inhibitor is, for example, a BRAF inhibitor. The chemotherapeutic agents can be administered alone or in combination (e.g., RAF inhibitors with MEK inhibitors). The cancer is any cancer in which the tumor has a B-RAF activating mutation. For example the cancer is melanoma, colon cancer, lung cancer, brain cancer, hematologic cancers or thyroid cancer. Definitions

“Sensitizing” a tumor cell to a chemotherapeutic agent, as used herein, refers to the act of enhancing the sensitivity of a tumor cell to a chemotherapeutic agent.

“Sensitivity” of a tumor cell to a chemotherapeutic agent is the susceptibility of the tumor cell to the inhibitory effect of the chemotherapeutic agent. For example, sensitivity of a tumor cell to a chemotherapeutic agent is indicated by reduction in growth rate of the cell in response to the chemotherapeutic agent. The sensitivity may also be demonstrated by a reduction of the symptoms caused by the neoplastic cells.

A tumor cell that is “refractory” to a chemotherapeutic agent is tumor cell not killed or growth inhibited by the chemotherapeutic agent. To determine if a tumor cell is growth inhibited, the growth rate of the cell in the presence or absence of the chemotherapeutic agent can be determined by established methods in the art. The tumor cell is not growth inhibited by the chemotherapeutic agent if the growth rate is not significantly different with or without the chemotherapeutic agent.

A tumor that is “refractory” to a chemotherapeutic agent is a tumor of which the rate of size increase or weight increase does not change in the presence of the chemotherapeutic agent. Alternatively, if the subject bearing the tumor displays similar symptoms or indicators of the tumor whether the subject receives the chemotherapeutic agent or not, the tumor is refractory to the chemotherapeutic agent.

A “tumor cell”, also known as a “cell with a proliferative disorder”, refers to a cell which proliferates at an abnormally high rate. A new growth comprising tumor cells is a tumor, also known as cancer. A tumor is an abnormal tissue growth, generally forming a distinct mass that grows by cellular proliferation more rapidly than normal tissue growth. A tumor may show partial or total lack of structural organization and functional coordination with normal tissue. As used herein, a tumor is intended to encompass hematopoietic tumors as well as solid tumors.

A tumor may be benign (benign tumor) or malignant (malignant tumor or cancer). Malignant tumors can be broadly classified into three major types. Malignant neoplasms arising from epithelial structures are called carcinomas, malignant neoplasms that originate from connective tissues such as muscle, cartilage, fat or bone are called sarcomas and malignant tumors affecting hematopoietic structures (structures pertaining to the formation of blood cells) including components of the immune system, are called leukemias and lymphomas.

A “proliferative disorder” is a disease or condition caused by cells which grow more quickly than normal cells, i.e., tumor cells. Proliferative disorders include benign tumors and malignant tumors. When classified by structure of the tumor, proliferative disorders include solid tumors and hematopoietic tumors.

“B-RAF-activated tumor cells” or “B-RAF-mediated tumor cells” refer to cells which proliferate at an abnormally high rate due to, at least in part, activation of the B-RAF which activated the downstream MAPK pathway. B-RAF may be activated by way of B-RAF gene structural mutation, elevated level of B-RAF gene expression, elevated stability of the B-RAF gene message, or any mutation or other mechanism which leads to the activation of B-RAF or a factor or factors downstream or upstream from B-RAF in the MAPK pathway, thereby increasing the MAPK pathway activity.

A “chemotherapeutic agent” or “chemotherapeutic drug” is any chemical compound used in the treatment of a proliferative disorder. Chemotherapeutic agents include, but are not limited to, RAF inhibitors (e.g., BRAF inhibitors), MEK inhibitors, PI3K inhibitors and AKT inhibitors. Other chemotherapeutic agents include, without being limited to, the following classes of agents: nitrogen mustards, e.g., cyclophosphamide, trofosfamide, ifosfamide and chlorambucil; nitroso ureas, e.g., carmustine (BCNU), lomustine (CCNU), semustine (methyl CCNU) and nimustine (ACNU); ethylene imines and methyl-melamines, e.g., thiotepa; folic acid analogs, e.g., methotrexate; pyrimidine analogs, e.g., 5-fluorouracil and cytarabine; purine analogs, e.g., mercaptopurine and azathioprine; vinca alkaloids, e.g., vinblastine, vincristine and vindesine; epipodophyllotoxins, e.g., etoposide and teniposide; antibiotics, e.g., dactinomycin, daunorubicin, doxorubicin, epirubicin, bleomycin a2, mitomycin c and mitoxantrone; estrogens, e.g., diethyl stilbestrol; gonadotropin-releasing hormone analogs, e.g., leuprolide, buserelin and goserelin; antiestrogens, e.g., tamoxifen and aminoglutethimide; androgens, e.g., testolactone and drostanolonproprionate; platinates, e.g., cisplatin and carboplatin; and interferons, including interferon-alpha, beta and gamma.

“Treating a proliferative disorder” means alleviating or eliminating the symptoms of a proliferative disorder, or slowing down the progress of a proliferative disorder.

A “metastatic tumor” is a tumor that has metastasized from a tumor located at another place in the same animal.

An “effective amount” is an amount of a chemotherapeutic agent or MET inhibitor which is sufficient to result in the intended effect. For a chemotherapeutic agent used to treat a disease, an efficient amount is an amount sufficient to alleviate or eliminate the symptoms of the disease, or to slow down the progress of the disease. For a MET inhibitor to sensitize (i.e. reduce or prevent acquired chemoresistance) a tumor to a chemotherapeutic agent, an efficient amount is an amount sufficient to increase sensitivity of the tumor to the chemotherapeutic agent.

“Progressive drug resistance” refers to the phenomenon wherein a tumor is initially susceptible to a chemotherapeutic agent, but the efficacy of the agent in inhibiting tumor growth or reducing symptoms of the disease decreases over time.

“Innate drug resistance” or “primary drug resistance” refers to the phenomenon wherein a tumor initially exhibits some resistance to a chemotherapeutic agent prior to any exposure to or treatment with said chemotherapeutic agent. This resistance may be conferred by or correlated to the presence of a mutation within the tumor, for example, the activating mutation BRAF V600E. This resistance may be conferred by or correlated to presence of or exposure to growth factors. For example, the resistance is conferred by exposure to growth factors secreted by stromal cells.

c-MET Kinase (MET) Inhibitors

A MET inhibitor is a compound that decreases the expression or activity of MET. As used herein the term MET inhibitors is meant to include ant agent that blocks the activity of Hepatocyte Growth Factor (HGF) or its cognate receptor c-MET.

MET is a membrane receptor that is essential for embryonic development and wound healing. Hepatocyte growth factor (HGF) is the only known ligand of the MET receptor. MET is normally expressed by cells of epithelial origin, while expression of HGF is usually restricted to cells of mesenchymal origin. Upon HGF stimulation, MET induces several biological responses that collectively give rise to a program known as invasive growth. Abnormal MET activation in cancer correlates with poor prognosis, where aberrantly active MET triggers tumor growth, formation of new blood vessels (angiogenesis) that supply the tumor with nutrients, and spread of the cancer to other organs (metastasis). MET is deregulated in many types of human malignancies, including cancers of kidney, liver, stomach, breast, and brain. Normally, only stem cells and progenitor cells express MET, which allows these cells to grow invasively in order to generate new tissues in an embryo or regenerate damaged tissues in an adult. However, cancer stem cells are thought to hijack the ability of normal stem cells to express MET, and thus become the cause of cancer persistence and spread to other sites in the body.

A decrease in MET expression or activity is defined by a reduction of a biological function of the tyrosine kinase. A biological function of a tyrosine kinase includes for example, catalyzing the phosphorylation of tyrosine.

A MET inhibitor acts for example by, blocking kinase-substrate interaction, inhibiting the enzyme's adenosine triphosphate (ATP) binding site, blocking extracellular tyrosine kinase receptors on cells or blocking HGF from binding MET.

MET kinase activity is measured by detecting phosphorylation of a protein. MET inhibitors are known in the art or are identified using methods described herein. For example, a MET inhibitor is identified by detecting a decrease the tyrosine kinase mediated transfer phosphate from ATP to protein tyrosine residues.

The MET inhibitor is, for example, a small molecule or a neutralizing antibody that inhibits MET kinase activity. The MET inhibitor is for example, a HGF neutralizing antibody (e.g., Ficlatuzumab) or a MET neutralizing antibody. The MET inhibitor is for example, a small molecule that inhibits HGF or MET.

Exemplary MET inhibitors include but are not limited to: (3Z)-5-(2,3-dihydro-1H-indol-1-ylsulfonyl)-3-({3,5-dimethyl-4-[(4-methylpiperazin-1-yl)carbonyl]-1H-pyrrol-2-yl}methylene)-1,3-dihydro-2H-indol-2-one, (3Z)-N-(3-chlorophenyl)-3-({3,5-dimethyl-4-[(4-methylpiperazin-1-yl)carbonyl]-1H-pyrrol-2-yl}methylene)-N-methyl-2-oxoindoline-5-sulfonamide, (3Z)-N-(3-chlorophenyl)-3-{[3,5-dimethyl-4-(3-morpholin-4-ylpropyl)-1H-pyrrol-2-yl]methylene}-N-methyl-2-oxoindoline-5-sulfonamide, AMG-208, AMG 337, Axitinib, Foretinib, JNJ-38877605, MGCD-265, PF-04217903, Crizotinib, Cabozantinib, PHA-665752, SGX-523, SU11274, XL184, ARQ197, XL880, INC280, Onartuzumab (MetMab), Trametinib, selumetinib, PD0325901, PD184,352, PHA-665752, JNJ-38877605, Rilotumumab or Ficlatuzumab.

Other MET inhibitors include those described in U.S. Pat. Nos. 7,872,031; 7,892,770; 7,803,907; 7,919,502; 7,250,417 and 7,037,909 each of which is hereby incorporated by reference in their entireties.

Therapeutic Methods

The growth of cells is inhibited, e.g., reduced by contacting a cell with a composition containing a MET inhibitor and a chemotherapeutic agent. By inhibition of cell growth is meant the cell proliferates at a lower rate or has decreased viability compared to a cell not exposed to the composition. Cell growth is measured by methods know in the art such as, the MTT cell proliferation assay, BrDU incorporation, immunohistochemical staining for proliferation markers or measurement of total GFP from GFP expressing cell lines.

Cells are directly contacted with an inhibitor. Alternatively, the inhibitor is administered systemically. In some aspects, the inhibitor is administered directly to or into the tumor cells or stromal cells. In some aspects, the inhibitor is administered near the tumor cells or stromal cells.

The cell is a tumor cell such as a carcinoma, adenocarcinoma, blastoma, leukemia, myeloma, or sarcoma. In particular, the cancer is melanoma, colon cancer, lung cancer, brain cancer, hematologic cancers or thyroid cancer or any other cancer harboring a BRAF activating mutation.

In various aspects the cell containing a B-RAF activating mutation. B-RAF activating mutations are identified by methods known in the art. The cell is resistant to B-RAF or MEK inhibitors when administered alone.

An exemplary B-RAF activating mutation is V600E.

The methods are useful to alleviate the symptoms of a variety of cancers. Any cancer containing a B-RAF activating mutation is amenable to treatment by the methods of the invention. In some aspects, the subject is suffering from melanoma or colon cancer.

Treatment is efficacious if the treatment leads to clinical benefit such as, a decrease in size, prevalence, or metastatic potential of the tumor in the subject. When treatment is applied prophylactically, “efficacious” means that the treatment retards or prevents tumors from forming or prevents or alleviates a symptom of clinical symptom of the tumor. Efficaciousness is determined in association with any known method for diagnosing or treating the particular tumor type.

Therapeutic Administration

The invention includes administering compositions comprising a chemotherapeutic agent and a MET inhibitor to a subject.

An effective amount of a therapeutic compound is preferably from about 0.1 mg/kg to about 150 mg/kg. Effective doses vary, as recognized by those skilled in the art, depending on route of administration, excipient usage, and coadministration with other therapeutic treatments including use of other anti-proliferative agents or therapeutic agents for treating, preventing or alleviating a symptom of a cancer. A therapeutic regimen is carried out by identifying a mammal, e.g., a human patient suffering from a cancer that has a BRAF activating mutation using standard methods.

The pharmaceutical compound is administered to such an individual using methods known in the art. Preferably, the compound is administered orally, rectally, nasally, topically or parenterally, e.g., subcutaneously, intraperitoneally, intramuscularly, and intravenously. The inhibitors are optionally formulated as a component of a cocktail of therapeutic drugs to treat cancers. Examples of formulations suitable for parenteral administration include aqueous solutions of the active agent in an isotonic saline solution, a 5% glucose solution, or another standard pharmaceutically acceptable excipient. Standard solubilizing agents such as PVP or cyclodextrins are also utilized as pharmaceutical excipients for delivery of the therapeutic compounds.

The therapeutic compounds described herein are formulated into compositions for other routes of administration utilizing conventional methods. For example, the therapeutic compounds are formulated in a capsule or a tablet for oral administration. Capsules may contain any standard pharmaceutically acceptable materials such as gelatin or cellulose. Tablets may be formulated in accordance with conventional procedures by compressing mixtures of a therapeutic compound with a solid carrier and a lubricant. Examples of solid carriers include starch and sugar bentonite. The compound is administered in the form of a hard shell tablet or a capsule containing a binder, e.g., lactose or mannitol, conventional filler, and a tableting agent. Other formulations include an ointment, suppository, paste, spray, patch, cream, gel, resorbable sponge, or foam. Such formulations are produced using methods well known in the art.

Therapeutic compounds are effective upon direct contact of the compound with the affected tissue. The compounds are administered into or near the tumor. Accordingly, the compound is administered topically. Alternatively, the therapeutic compounds are administered systemically. In some aspects, the compounds are administered by inhalation. The compounds are delivered in the form of an aerosol spray from pressured container or dispenser which contains a suitable propellant, e.g., a gas such as carbon dioxide, or a nebulizer.

Additionally, compounds are administered by implanting (either directly into an organ, tumor, or subcutaneously) a solid or resorbable matrix which slowly releases the compound into adjacent and surrounding tissues of the subject.

Diagnostic Methods

Chemoresistance or a predisposition thereto is detected by examining the expression HGF or MET activation from a test population of cells (i.e., a patient derived tissue sample). A tissue sample is for example, a biopsy tissue, scrapings, or tumor tissue removed during surgery.

Expression of HGF or MET activation is determined in the test sample and compared to the expression of the normal control level. By normal control level is meant the expression level of HGF or MET activation typically found in a population not suffering from a tumor. The normal control level can be a range or an index. Alternatively, the normal control level can be a database of expression patterns from previously tested individuals. An increase of the level of expression of HGF or MET activation in the patient derived sample indicates that the subject is chemoresistant or is at risk of developing chemoresistance.

Expression of HGF is determined by detecting an HGF polypeptide or nucleic acid, e.g., RNA or DNA. MET activation is determined for example by detection MET phosporylation

Expression of HGF or MET activation also allows for the course of treatment of the tumors to be monitored. In this method, a biological sample is provided from a subject undergoing treatment, e.g., surgical, chemotherapeutic or hormonal treatment. If desired, biological samples are obtained from the subject at various time points before, during, or after treatment. Expression of HGF or MET activation is then determined and compared to a reference, e.g. control whose chemoresistant state is known. The reference sample has been exposed to the treatment. Alternatively, the reference sample has not been exposed to the treatment. Optionally, such monitoring is carried out preliminary at second look surgical surveillance procedures and subsequent surgical surveillance procedures. For example, samples may be collected from subjects who have received initial surgical treatment for cancer and subsequent treatment with antineoplastic agents for that cancer to monitor the development of chemoresistance.

Expression of HGF is determined at the protein or nucleic acid level using any method known in the art. For example. Northern hybridization analysis using probes which specifically recognize one or more of these sequences can be used to determine gene expression. Alternatively, expression is measured using reverse-transcription-based PCR assays, e.g., using primers specific for the differentially expressed sequence of genes. Transcriptional profiling using cDNA microarray chips may also be used to measure expression of HGF. Expression is also determined at the protein level, i.e., by measuring the levels of polypeptides encoded by the gene products described herein, or activities thereof. Such methods are well known in the art and include, e.g., immunoassays based on antibodies to proteins encoded by the genes. Any biological material can be used for the detection/quantification of the protein or its activity. Alternatively, a suitable method can be selected to determine the activity of proteins encoded by the marker genes according to the activity of each protein analyzed.

MET activation is determined by methods know in the art, for example by detecting phosphorylation of MET.

The difference in the level of HGF or MET activation in the control sample compared to the test sample is statistically significant. By statistically significant is meant that the alteration is greater than what might be expected to happen by chance alone. Statistical significance is determined by method known in the art. For example statistical significance is determined by p-value. The p-values are a measure of probability that a difference between groups during an experiment happened by chance. (P(z≧z_{observed})). For example, a p-value of 0.01 means that there is a 1 in 100 chance the result occurred by chance. The lower the p-value, the more likely it is that the difference between groups was caused by treatment. An alteration is statistically significant if the p-value is at least 0.05. Preferably, the p-value is 0.04, 0.03, 0.02, 0.01, 0.005, 0.001 or less.

The “diagnostic accuracy” of a test, assay, or method concerns the ability of the test, assay, or method to distinguish between patients a chemoresistant tumor or at risk for developing a chemoresistant tumor is based on whether the patients have a “clinically significant presence” of HGF or MET activation. By “clinically significant presence” is meant that the presence of the HGF in the patient (typically in a sample from the patient) is higher than the predetermined cut-off point (or threshold value) for HGF or MET activation and therefore indicates that the patient has a chemoresistant tumor.

The terms “high degree of diagnostic accuracy” and “very high degree of diagnostic accuracy” refer to the test or assay for HGF or MET activation with the predetermined cut-off point correctly (accurately) indicating the presence or absence of chemoresistance. A perfect test would have perfect accuracy. Thus, for individuals who have chemoresistance the test would indicate only positive test results and would not report any of those individuals as being “negative” (there would be no “false negatives”). In other words, the “sensitivity” of the test (the true positive rate) would be 100%. On the other hand, for individuals who were not chemoresistant, the test would indicate only negative test results and would not report any of those individuals as being “positive” (there would be no “false positives”). In other words, the “specificity” (the true negative rate) would be 100%. See, e.g., O'Marcaigh A S. Jacobson R M, “Estimating The Predictive Value Of A Diagnostic Test, How To Prevent Misleading Or Confusing Results,” Clin. Ped. 1993, 32(8): 485-491, which discusses specificity, sensitivity, and positive and negative predictive values of a test, e.g., a clinical diagnostic test.

Changing the cut point or threshold value of a test (or assay) usually changes the sensitivity and specificity but in a qualitatively inverse relationship. For example, if the cut point is lowered, more individuals in the population tested will typically have test results over the cut point or threshold value. Because individuals who have test results above the cut point are reported as having the disease, condition, or syndrome for which the test is being run, lowering the cut point will cause more individuals to be reported as having positive results. Thus, a higher proportion of those who have a chemoresistance will be indicated by the test to have it. Accordingly, the sensitivity (true positive rate) of the test will be increased. However, at the same time, there will be more false positives because more people who do not have the disease, condition, or syndrome (i.e., people who are truly “negative”) will be indicated by the test to have HGF or MET activation values above the cut point and therefore to be reported as positive (i.e., to have the disease, condition, or syndrome) rather than being correctly indicated by the test to be negative. Accordingly, the specificity (true negative rate) of the test will be decreased. Similarly, raising the cut point will tend to decrease the sensitivity and increase the specificity. Therefore, in assessing the accuracy and usefulness of a proposed medical test, assay, or method for assessing a patient's condition, one should always take both sensitivity and specificity into account and be mindful of what the cut point is at which the sensitivity and specificity are being reported because sensitivity and specificity may vary significantly over the range of cut points.

There is, however, an indicator that allows representation of the sensitivity and specificity of a test, assay, or method over the entire range of cut points with just a single value. That indicator is derived from a Receiver Operating Characteristics (“ROC”) curve for the test, assay, or method in question. See, e.g., Shultz, “Clinical Interpretation Of Laboratory Procedures,” chapter 14 in Teitz, Fundamentals of Clinical Chemistry, Burtis and Ashwood (eds.), 4th edition 1996, W.B. Saunders Company, pages 192-199; and Zweig et al., “ROC Curve Analysis: An Example Showing The Relationships Among Serum Lipid And Apolipoprotein Concentrations In Identifying Patients With Coronory Artery Disease,” Clin. Chem., 1992, 38(8): 1425-1428.

An ROC curve is an x-y plot of sensitivity on the y-axis, on a scale of zero to one (i.e., 100%), against a value equal to one minus specificity on the x-axis, on a scale of zero to one (i.e., 100%). In other words, it is a plot of the true positive rate against the false positive rate for that test, assay, or method. To construct the ROC curve for the test, assay, or method in question, patients are assessed using a perfectly accurate or “gold standard” method that is independent of the test, assay, or method in question to determine whether the patients are truly positive or negative for the disease, condition, or syndrome. The patients are also tested using the test, assay, or method in question, and for varying cut points, the patients are reported as being positive or negative according to the test, assay, or method. The sensitivity (true positive rate) and the value equal to one minus the specificity (which value equals the false positive rate) are determined for each cut point, and each pair of x-y values is plotted as a single point on the x-y diagram. The “curve” connecting those points is the ROC curve.

The area under the curve (“AUC”) is the indicator that allows representation of the sensitivity and specificity of a test, assay, or method over the entire range of cut points with just a single value. The maximum AUC is one (a perfect test) and the minimum area is one half. The closer the AUC is to one, the better is the accuracy of the test.

By a “high degree of diagnostic accuracy” is meant a test or assay (such as the test of the invention for determining the clinically significant presence of HGF or MET activation, which thereby indicates the presence of chemoresistance) in which the AUC (area under the ROC curve for the test or assay) is at least 0.70, desirably at least 0.75, more desirably at least 0.80, preferably at least 0.85, more preferably at least 0.90, and most preferably at least 0.95.

By a “very high degree of diagnostic accuracy” is meant a test or assay in which the AUC (area under the ROC curve for the test or assay) is at least 0.875, desirably at least 0.90, more desirably at least 0.925, preferably at least 0.95, more preferably at least 0.975, and most preferably at least 0.98.

The subject is preferably a mammal. The mammal is, e.g., a human, non-human primate, mouse, rat, dog, cat, horse, or cow.

Diagnostic kits for carrying out the methods described herein are produced in a number of ways. In one embodiment, the diagnostic kit comprises (a) an antibody (e.g., HGF) conjugated to a solid support and (b) a second antibody of the invention conjugated to a detectable group. The reagents may also include ancillary agents such as buffering agents and protein stabilizing agents, e.g., polysaccharides and the like. The diagnostic kit may further include, where necessary, other members of the signal-producing system of which system the detectable group is a member (e.g., enzyme substrates), agents for reducing background interference in a test, control reagents, apparatus for conducting a test, and the like. Alternatively, a test kit contains (a) an antibody, and (b) a specific binding partner for the antibody conjugated to a detectable group. Ancillary agents as described above may likewise be included. The test kit may be packaged in any suitable manner, typically with all elements in a single container along with a sheet of printed instructions for carrying out the test.

### EXAMPLES

### Example 1

### Identification of the Mechanism that Underlies Stroma-Mediated Primary Chemoresistance

Metastatic melanoma is an aggressive skin cancer with incidence that doubles roughly every decade in western countries. Moreover, 50-70% of melanoma patients have an activating, typically V600E, mutation in the serine/threonine kinase BRAF. The constitutively activated B-RAF activates MEK and ERK downstream in the mitogen-activated protein kinase (MAPK) signaling pathway. Pre-clinical trials have shown that many of these V600E B-RAF melanoma cell lines are extremely sensitive to the V600E RAF inhibitors PLX4720 and PLX4032 (Vemurafenib). Recent clinical trials using PLX4032 on a stratified group of patients with the V600E B-RAF mutation showed substantial activity against these aggressive tumors. Unfortunately, most patients exhibit only a partial response to the drug, after which progression of tumor growth eventually continues in almost all treated patients.

A high-throughput screen data identified a subset of 6 fibroblast cell lines that allow melanoma cell lines with the V600E B-RAF mutation to proliferate under continuous treatment with PLX4720 or with the MEK inhibitor PD184352. This stromal-induced drug resistance is strikingly different from two recently published studies that investigated the mechanisms underlying BRAF inhibitor resistance. Previous studies selected melanoma cell lines for many months before resistant cell lines were established. In contrast, the fibroblasts in the co-culture system disclosed herein conferred immediate, up-front primary resistance to the melanoma cell lines, allowing proliferation under continuous drug treatment. As culturing the melanoma cell lines with media from these 6 fibroblast cell lines was sufficient to induce resistance, it was concluded that a factor secreted by the fibroblast cells is responsible for this fibroblast-induced drug resistance.

### EXAMPLE 2

### Identification of Hepatocyte Growth Factor as the Mediator of Stroma Mediated Drug Resistance

In order to identify the secreted factor that promoted the stroma-mediated drug resistance, two types of antibody arrays were used to measure 274 and 507 cytokines, chemokines, adipokines, growth factors, angiogenic factors, proteases, soluble receptors, soluble adhesion molecules and other proteins in the media of 18 stromal cell lines, searching for proteins that are uniquely secreted by the resistance-inducing stromal cells. The top ranking protein in both experiments was found to be hepatocyte growth factor (HGF), and its secretion levels in all cell lines were further validated by ELISA. HGF is a paracrine cellular growth factor that is secreted by mesenchymal cells and acts primarily upon epithelial cells by activating the proto-oncogenic tyrosine kinase receptor (RTK) c-MET (MET). While MET is known to be involved in the progression of melanoma, its role in BRAF inhibitor resistance has not been previously explored.

### EXAMPLE 3

### HGF Renders Melanoma Cells Line Resistant to BRAF/MEK Inhibitors

The addition of recombinant HGF to V600E BRAF melanoma cell lines is enough to confer BRAF/MEK inhibitor resistance. Furthermore, this acquired chemoresistance can be directly reversed by the addition of anti-HGF neutralizing antibodies or by Crizotinib—a small molecule that specifically inhibits the RTKs MET and ALK (Anaplastic Lymphoma Kinase).

The complexity of the tumor microenvironment is much greater than an in vitro co-culture system. Therefore, the inventors explored whether the activation of other RTKs could result in a similar resistance as that observed with the activation of MET. To this end, six V600E BRAF melanoma cell lines were tested for their resistance to either BRAFi (PLX4720) or MEKi (PD184352) after the addition of 22 RTK ligands that have the potential of activating almost all known RTKs. HGF was the only RTK ligand of those tested that could confer substantial primary resistance to the melanoma cell lines. Though not all RTKs are expressed on each melanoma cell line, expression profiling of the cell lines showed many of them to be expressed. The activation of a selected subset of these RTKs was confirmed by western blotting and by high throughput tyrosine kinase phosphorylation profiling. Interestingly, PDGF-BB and IGF-1, the ligands of PDGFRB and IGF-1R that were previously shown to be involved in acquired resistance to BRAF inhibition, were not shown to induce primary resistance during the experimental time course.

### EXAMPLE 4

### Molecular Basis for HGF-Induced Primary Resistance

HGF was shown to re-activate ERK only under PLX4720 treatment much more than under PD184352 treatment. Thus, MET can re-activate MEK through RAF1 (CRAF) while under BRAF inhibition (PLX-4720), however, MEK cannot be reactivated under direct MEK inhibition (PD184352). Therefore, PI3K/AKT signaling may be the MET downstream effectors that confer HGF-induced resistance under MEK inhibition. Under BRAF/MEK inhibitor treatment, pAKT is partially inhibited, but can be completely reactivated under HGF. This AKT-mediated resistance indicates that resistance from MAPK pathway inhibition does not rely solely upon ERK reactivation, but might be explained by activation of the PI3K/AKT pathway, as recently suggested by others as possible mechanisms of acquired resistance. This model predicts that both the MAPK pathway and the PI3K/AKT pathway can contribute to the primary resistance induced by HGF. In agreement with this model, the examples of the present invention show: 1) The HGF-induced resistance is greater under BRAF inhibition than under MEK inhibition, as both pathways can be activated by MET only under BRAF inhibition; 2) While combining MEK and AKT inhibition is enough to suppress all HGF-induced resistance, HGF can still induce some resistance under a combination of BRAF and AKT inhibitors by activating ERK; and 3) HGF-induced resistance was not observed under a combination of ERK and AKT inhibition, implying that no other pathway affected by HGF has a stand alone contribution to the HGF-induced resistance.

To understand why, of all the RTKs tested, only HGF could induce such a unique primary resistance phenotype, a high-throughput western analysis was used to test the ability of all 22 RTK ligands to re-activate pERK/pAKT under BRAFi (PLX4720) treatment in six V600E BRAF melanoma cell lines. HGF was the only cytokine able to reactivate both ERK and AKT after 1 hour of cytokine treatment. EGF, FGF-1 and PDGF-BB could reactivate only ERK, while insulin and IGF could only reactivate AKT. However, whereas the activation of both ERK and AKT by HGF under PLX4720 treatment was long lasting, the activation of either ERK or AKT by the above cytokines was only transient and thus could not confer resistance to the melanoma cell lines.

### EXAMPLE 5

### HGF Expression in BRAF V600E Melanoma Patient-Derived Biopsies

HGF expression was determined by immunohistochemistry (IHC) in 34 BRAF V600E melanoma patient-derived biopsies taken just prior to treatment with RAF inhibitor (or a combination of BRAF and MEK inhibitors). HGF was detected in the tumor-associated stromal cells in 23/34 patients (68%), and phospho-MET immunofluorescence studies similarly documented MET phosphorylation (activation) in patient samples.

The in vitro studies disclosed herein predict that the presence of stromal HGF is associated with innate resistance. Indeed, patients with stromal HGF had a significantly poorer response to treatment compared to those lacking expression (P<0.05). Interestingly, only one of the 34 patients had a durable complete response (14 months and continuing), and this patient lacked HGF expression. On-treatment biopsies taken 2 weeks after treatment initiation were also available from 10 patients, and for 5 of those (50%), stromal HGF expression was found to be increased compared to pre-treatment. This increase can be attributed to recruitment of HGF-secreting fibroblasts to the tumor and/or up-regulation of HGF in existing fibroblasts. Of note, both normal skin and benign nevi exhibited stromal HGF expression. These results thus support the clinical relevance of HGF-mediated resistance to BRAF inhibitors.

### EXAMPLE 6

### HGF-Mediated Innate Resistance to RAF Inhibitors in BRAF V600E Colorectal and Glioblastoma Cancers

Activation of the EGF receptor was recently shown to drive the resistance of some BRAF V600E colorectal cancer cell lines to RAF inhibition. In order to explore a possible role for MET activation in BRAF-mutant non-melanoma cancers, seven non-melanoma BRAF-mutant cell lines (5 colorectal and 2 glioblastoma) were tested, and all 7 cell lines had evidence of MET expression and phoshorylation. Although stromal HGF expression is less common in colorectal cancer compared to melanoma, MET overexpression and HGF autocrine secretion have been documented in colorectal cancer. Two HGF-secreting, BRAF-mutant non-melanoma cell lines (one colorectal (RKO) and one glioblastoma (KG-1-C)) were identified. In these cell lines, combined RAF and MET (but not EGFR) inhibition resulted in a clear synergistic effect. Synergy between BRAF and MET inhibitors was more variable among non-HGF-secreting BRAF-mutant cell lines. As predicted by the proposed mechanism of resistance, mono-therapy with BRAF or MEK inhibitors had no effect on pAKT and caused little inhibition of pERK in HGF-secreting cell lines. However, dual inhibition of BRAF and MET resulted in significant inhibition of both pERK and pAKT.

TABLE 1
Cancer cell lines screened

| | | |
|---|---|---|
| NSCLC | 12 | Calu-3/HCC2279/HCC2935/HCC4006/ |
| | | HCC827/NCI-H3122/NCI-H322M/NCI-H3255/ |
| | | NCI-H358/NCI-H596/NCI-H661/PC-9 |
| Breast | 9 | BT-474/EFM192A/HCC1419/HCC1569/ |
| | | MDA-MB-175-VII/MDA-MB-361/SK-BR-3/ |
| | | ZR-75-1/MCF-7 |
| Melanoma | 7 | A2058/C32/COLO 829/G-361/MALME-3M/ |
| | | SK-MEL-28/SK-MEL-5 |
| CRC | 8 | Colo-205/DLD-1/HCT-116/LS411N/HT-29/ |
| | | RKO/SW1417/WiDr |
| HNSCC | 5 | Cal27/FaDu/SCC-15/SCC-25/SCC-9 |
| Pancreatic | 3 | AsPC-1/BxPC-3/CFPAC-1 |
| GIST | 2 | GIST-882/GIST-T1 |
| Total: | 46 | |

TABLE 2A
Chemotherapy drugs and their mechanisms

| Drug | Mechanism |
|---|---|
| Carboplatin | Alkylating-like agent |
| Oxaliplatin | Alkylating-like agent |
| Gemcitabine | Antimetabolite (ribonucleotide) |
| Methotrexate | Antimetabolite (folic acid metabolism) |
| 5-Fluorouracil | Antimetabolite (Thymidylate synthase) |
| Vincristine | Anti-mitotic (Microtubule assembly) |
| Vinorelbine | Anti-mitotic (Microtubule assembly) |
| Docetaxel | Anti-mitotic (Microtubule disassembly) |
| Paclitaxel | Anti-mitotic (Microtubule disassembly) |
| Irinotecan | Topoisomerase I inhibitor |
| Etoposide | Topoisomerase II inhibitor |
| Doxorubicin | DNA Intercalation + Topoisomerase II inhibition |

TABLE 2B
Chemotherapy drugs and their targets

| Drug | Target |
|---|---|
| Gefitinib | EGFR |
| Erlotinib | EGFR |
| CL-387,785 | EGFR |
| BIBW2992 | EGFR |
| Lapatinib | EGFR/HER2 |
| Canertinib | EGFR/HER-2/ErbB-4 |
| Fulvestrant | ER |
| NVP-TAE684 | ALK |
| Crizotinib | MET/ALK |
| PLX4720 | BRAF |
| SB590885 | BRAF |
| AZD6244 | MEK |
| PD184352 | MEK |
| Imatinib | BCR-ABL, c-KIT, PDGFR |
| Dasatinib | BCR-ABL, Src |
| Sunitinib | VEGFR, PDGFR, c-KIT, FLT3, FGFR |
| Vandetanib | VEGFR-2/EGFR |
| BEZ235 | PI3K/mTOR |
| PI-103 | PI3Kalpha/mTOR |
| Bortezomib | Proteosome |
| Vorinostat | HDAC |
| ABT-263 | Bcl-2 |

TABLE 3
Stromal cells screened

| | |
|---|---|
| HS-27A | Bone marrow stroma |
| HS-5 | Bone marrow stroma |
| HMF | Breast - Mammary fibroblasts |
| Hs 343.T | Breast - Mammary CAFs |
| PC87322A1 | Breast - Mammary CAFs |
| PC87399A1 | Breast - Mammary CAFs |
| CCD-13Lu | Lung |
| CCD-8Lu | Lung |
| Hs888Lu | Lung |
| LL 86 | Lung |
| Wi-38 | Lung |
| PC87985B1 | Lung CAFs (NSCLC, Adeno) |
| PC60163A1 | Lung CAFs (NSCLC, Squamous) |
| CCD-1090Sk | Skin (Abdomen) |
| AG09877 | Skin (Arm) |
| CCD-1065SK | Skin (breast) |
| CCD-1068Sk | Skin (breast) |
| CCD-1069Sk | Skin (breast) |
| CCD-1117Sk | Skin (face) |
| HDF | Skin (upper abdomen) |
| HUVEC-CS | Umbilical vein endothelial (newborn) |
| 3T3-L1 | Adipocytes |

TABLE 4
Screening of the ability of recombinant RTK ligands to confer resistance to 6 melanoma cell lines.

| | | | | | Colo | | SK-MEL- | SK-MEL- | | | Colo | | SK-MEL- | |
|---|---|---|---|---|---|---|---|---|---|---|---|---|---|---|
| | | | A2058 | C32 | 829 | G-361 | 28 | 5 | A2058 | C32 | 829 | G-361 | 28 | SK-MEL-5 |
| | | | PLX4720 - 2 uM | | | | | | PD184352 - 1 uM | | | | | |
| Angiopoietin-1 | 1.56 | ng/ml | 1.02 | 1.20 | 0.93 | 1.02 | 1.05 | 1.05 | 1.02 | 1.08 | 0.94 | 0.98 | 1.09 | 1.03 |
| | 6.25 | ng/ml | 1.02 | 1.09 | 0.97 | 0.96 | 0.96 | 1.02 | 1.03 | 1.07 | 0.97 | 0.96 | 1.04 | 0.93 |
| | 25 | ng/ml | 0.91 | 1.14 | 0.95 | 0.97 | 1.02 | 0.98 | 0.96 | 1.02 | 0.97 | 0.92 | 1.12 | 0.95 |
| | 100 | ng/ml | 0.97 | 1.08 | 0.94 | 0.83 | 0.96 | 1.03 | 0.99 | 1.07 | 0.93 | 0.95 | 0.98 | 0.92 |
| | 400 | ng/ml | 0.96 | 0.99 | 1.05 | 0.84 | 0.99 | 1.04 | 0.90 | 0.98 | 0.97 | 0.75 | 0.96 | 0.87 |
| BDNF | 0.4 | ng/ml | 1.00 | 1.09 | 1.02 | 0.98 | 0.97 | 1.07 | 0.99 | 1.03 | 1.03 | 1.09 | 1.04 | 1.01 |
| | 1.6 | ng/ml | 0.95 | 0.99 | 1.00 | 0.82 | 1.12 | 1.02 | 0.97 | 1.01 | 0.99 | 0.95 | 1.04 | 0.97 |
| | 6.25 | ng/ml | 0.95 | 1.02 | 0.96 | 1.04 | 0.99 | 1.07 | 0.94 | 1.06 | 0.98 | 1.03 | 0.91 | 1.01 |
| | 25 | ng/ml | 0.98 | 1.01 | 0.95 | 0.89 | 1.08 | 1.08 | 1.03 | 1.03 | 0.98 | 0.91 | 0.97 | 0.96 |
| | 100 | ng/ml | 1.15 | 0.94 | 0.94 | 0.87 | 0.99 | 1.04 | 1.10 | 0.95 | 0.93 | 0.91 | 1.07 | 0.97 |
| Collagen II | 6.25 | ng/ml | 0.95 | 1.03 | 1.00 | 0.97 | 0.94 | 1.03 | 0.97 | 0.99 | 0.95 | 0.96 | 0.98 | 0.96 |
| | 25 | ng/ml | 0.99 | 0.99 | 1.13 | 0.91 | 1.12 | 1.10 | 0.97 | 1.03 | 1.02 | 1.00 | 1.10 | 0.92 |
| | 100 | ng/ml | 0.96 | 0.99 | 1.06 | 0.92 | 0.98 | 1.06 | 0.99 | 1.06 | 1.04 | 0.96 | 0.98 | 0.94 |
| | 400 | ng/ml | 0.95 | 1.00 | 1.02 | 0.96 | 0.95 | 1.00 | 1.00 | 1.18 | 1.02 | 1.01 | 0.94 | 0.99 |
| | 1600 | ng/ml | 0.96 | 1.02 | 0.98 | 0.92 | 0.96 | 1.08 | 0.92 | 1.14 | 0.99 | 1.03 | 1.07 | 0.94 |
| EGF | 0.4 | ng/ml | 1.02 | 1.06 | 1.02 | 0.98 | 1.00 | 1.08 | 1.05 | 1.01 | 0.97 | 1.01 | 1.03 | 0.99 |
| | 1.6 | ng/ml | 1.03 | 0.94 | 1.00 | 0.90 | 1.01 | 1.15 | 1.04 | 0.97 | 0.96 | 0.90 | 0.97 | 1.04 |
| | 6.25 | ng/ml | 0.93 | 1.03 | 1.00 | 0.94 | 0.97 | 1.19 | 0.95 | 1.06 | 0.99 | 0.97 | 0.93 | 1.08 |
| | 25 | ng/ml | 0.97 | 0.93 | 0.97 | 0.99 | 0.94 | 1.22 | 0.96 | 1.01 | 0.94 | 1.00 | 0.97 | 1.11 |
| | 100 | ng/ml | 0.99 | 0.99 | 1.01 | 0.93 | 1.03 | 1.11 | 1.00 | 0.99 | 1.00 | 0.96 | 1.05 | 1.01 |
| Ephrin-A4 | 1.6 | ng/ml | 0.97 | 1.04 | 1.06 | 1.08 | 1.10 | 0.91 | 1.00 | 1.02 | 1.08 | 1.11 | 0.98 | 0.96 |
| | 6.25 | ng/ml | 0.99 | 1.01 | 1.19 | 1.05 | 0.84 | 1.07 | 1.02 | 1.02 | 1.13 | 1.09 | 0.88 | 0.93 |
| | 25 | ng/ml | 0.96 | 1.04 | 1.00 | 0.95 | 1.05 | 0.99 | 0.99 | 0.98 | 1.01 | 1.04 | 0.99 | 0.93 |
| | 100 | ng/ml | 0.99 | 1.10 | 0.99 | 1.02 | 1.10 | 0.99 | 1.04 | 1.04 | 0.98 | 0.98 | 1.07 | 0.98 |
| | 400 | ng/ml | 0.98 | 1.03 | 0.90 | 0.88 | 1.00 | 0.93 | 1.07 | 0.90 | 0.96 | 0.86 | 0.90 | 0.99 |
| FGF1 | 0.4 | ng/ml | 1.02 | 1.11 | 0.99 | 0.96 | 1.00 | 1.01 | 1.04 | 1.01 | 1.02 | 0.99 | 1.05 | 0.98 |
| | 1.6 | ng/ml | 1.01 | 1.10 | 1.09 | 0.95 | 1.02 | 1.12 | 1.05 | 1.04 | 1.11 | 1.09 | 0.87 | 1.07 |
| | 6.25 | ng/ml | 0.96 | 1.08 | 1.02 | 0.92 | 1.10 | 1.12 | 1.04 | 1.03 | 1.00 | 0.99 | 0.90 | 1.14 |
| | 25 | ng/ml | 1.05 | 0.93 | 1.07 | 0.89 | 0.98 | 1.09 | 1.12 | 1.02 | 1.08 | 1.08 | 0.92 | 1.17 |
| | 100 | ng/ml | 1.11 | 1.00 | 1.06 | 1.08 | 1.08 | 1.28 | 1.19 | 0.97 | 1.12 | 1.04 | 1.06 | 1.36 |
| flt-3 ligand | 0.4 | ng/ml | 0.97 | 0.99 | 1.08 | 1.00 | 0.94 | 1.07 | 1.03 | 1.02 | 1.07 | 1.04 | 1.04 | 0.97 |
| (FL) | 1.6 | ng/ml | 0.98 | 0.97 | 0.98 | 0.87 | 0.90 | 1.07 | 0.99 | 1.08 | 0.98 | 1.09 | 0.95 | 0.97 |
| | 6.25 | ng/ml | 1.01 | 1.00 | 1.00 | 0.88 | 0.86 | 1.03 | 1.00 | 1.10 | 0.97 | 0.97 | 0.98 | 1.00 |
| | 25 | ng/ml | 1.06 | 0.87 | 1.05 | 0.88 | 0.82 | 0.93 | 1.02 | 1.08 | 1.05 | 1.01 | 0.78 | 0.92 |
| | 100 | ng/ml | 0.93 | 0.97 | 0.97 | 0.79 | 0.98 | 1.04 | 0.89 | 1.07 | 0.97 | 0.89 | 0.99 | 0.90 |
| Gas6 | 6.25 | ng/ml | 1.01 | 1.05 | 0.97 | 0.97 | 1.13 | 0.98 | 0.99 | 1.02 | 0.98 | 1.03 | 1.03 | 0.99 |
| | 25 | ng/ml | 1.00 | 0.89 | 1.02 | 0.95 | 0.96 | 1.00 | 0.97 | 1.01 | 1.01 | 1.00 | 0.87 | 1.01 |
| | 100 | ng/ml | 0.98 | 1.00 | 0.94 | 0.98 | 1.04 | 1.03 | 1.02 | 1.01 | 0.98 | 0.91 | 1.00 | 0.97 |
| | 400 | ng/ml | 0.98 | 1.05 | 0.99 | 0.97 | 1.08 | 1.05 | 0.97 | 1.15 | 0.99 | 0.94 | 1.01 | 0.89 |
| | 1600 | ng/ml | 0.97 | 0.95 | 1.03 | 0.85 | 1.22 | 0.97 | 0.95 | 0.91 | 1.02 | 0.88 | 1.14 | 0.94 |
| GDNF | 0.4 | ng/ml | 0.97 | 1.04 | 1.00 | 0.94 | 1.04 | 1.04 | 0.98 | 1.08 | 0.95 | 1.00 | 0.99 | 0.99 |
| | 1.6 | ng/ml | 0.97 | 1.11 | 1.00 | 0.94 | 0.91 | 0.71 | 1.03 | 1.13 | 0.96 | 0.94 | 1.03 | 0.89 |
| | 6.25 | ng/ml | 0.98 | 1.03 | 0.96 | 0.93 | 1.05 | 1.00 | 1.00 | 1.03 | 0.96 | 1.04 | 1.02 | 1.03 |
| | 25 | ng/ml | 0.94 | 0.91 | 1.09 | 0.87 | 0.97 | 1.01 | 0.93 | 0.96 | 1.07 | 0.92 | 0.92 | 0.94 |
| | 100 | ng/ml | 0.97 | 0.94 | 1.00 | 0.79 | 1.01 | 1.08 | 0.92 | 0.94 | 0.98 | 0.95 | 0.96 | 0.93 |
| HGF | 0.4 | ng/ml | 1.03 | 1.04 | 1.00 | 0.97 | 1.05 | 0.98 | 1.02 | 1.10 | 0.96 | 1.17 | 1.11 | 0.99 |
| | 1.6 | ng/ml | 1.02 | 1.15 | 1.02 | 1.03 | 1.42 | 1.37 | 1.01 | 1.08 | 0.99 | 1.03 | 1.16 | 1.17 |
| | 6.25 | ng/ml | 1.12 | 1.42 | 1.14 | 1.24 | 1.55 | 1.35 | 1.10 | 1.29 | 1.12 | 1.19 | 1.22 | 1.16 |
| | 25 | ng/ml | 1.32 | 1.96 | 1.35 | 2.27 | 3.85 | 1.95 | 1.10 | 1.08 | 1.41 | 1.68 | 1.46 | 1.81 |
| | 100 | ng/ml | 1.49 | 2.01 | 1.71 | 9.24 | 2.20 | 1.93 | 1.66 | 1.96 | 1.89 | 1.10 | 1.87 | 2.32 |
| IGF-I | 0.4 | ng/ml | 1.05 | 1.08 | 1.00 | 1.03 | 1.24 | 1.01 | 1.10 | 1.05 | 1.06 | 1.06 | 1.10 | 1.01 |
| | 1.6 | ng/ml | 0.95 | 1.12 | 0.99 | 1.05 | 1.08 | 1.03 | 1.02 | 1.04 | 0.97 | 1.00 | 1.06 | 1.01 |
| | 6.25 | ng/ml | 0.97 | 1.17 | 1.06 | 1.11 | 1.09 | 1.19 | 1.03 | 1.21 | 1.03 | 1.17 | 0.98 | 1.08 |
| | 25 | ng/ml | 1.04 | 1.15 | 1.03 | 0.99 | 1.02 | 1.25 | 1.08 | 1.16 | 1.01 | 0.99 | 1.06 | 1.06 |
| | 100 | ng/ml | 0.99 | 1.07 | 1.07 | 0.87 | 1.01 | 1.12 | 1.02 | 1.21 | 1.08 | 0.95 | 1.12 | 1.29 |
| Insulin | 156.25 | ng/ml | 0.98 | 1.14 | 0.91 | 0.92 | 1.02 | 1.10 | 0.98 | 1.09 | 0.91 | 0.98 | 1.01 | 1.20 |
| | 625 | ng/ml | 0.96 | 1.05 | 0.93 | 0.88 | 0.95 | 1.18 | 0.99 | 1.08 | 0.98 | 0.91 | 0.97 | 1.24 |
| | 2500 | ng/ml | 1.12 | 1.20 | 0.97 | 0.98 | 0.99 | 1.27 | 1.14 | 1.20 | 0.98 | 1.01 | 1.06 | 1.30 |
| | 10000 | ng/ml | 1.02 | 1.02 | 1.01 | 0.96 | 1.05 | 1.26 | 1.03 | 1.08 | 1.02 | 0.98 | 1.13 | 1.28 |
| | 40000 | ng/ml | 1.11 | 1.04 | 1.00 | 0.99 | 1.05 | 1.43 | 1.06 | 1.04 | 1.02 | 0.97 | 1.11 | 1.66 |
| MSP | 0.4 | ng/ml | 1.04 | 1.07 | 0.98 | 1.00 | 0.92 | 0.87 | 1.06 | 0.99 | 1.03 | 1.04 | 0.95 | 0.94 |
| | 1.6 | ng/ml | 1.02 | 1.01 | 0.94 | 0.89 | 1.02 | 1.03 | 0.98 | 1.02 | 0.98 | 0.96 | 0.94 | 1.11 |
| | 6.25 | ng/ml | 1.05 | 1.04 | 1.03 | 0.91 | 1.00 | 0.98 | 1.03 | 1.07 | 1.01 | 0.92 | 1.00 | 1.00 |
| | 25 | ng/ml | 1.04 | 1.02 | 0.97 | 0.93 | 0.97 | 1.01 | 1.09 | 1.01 | 0.96 | 0.98 | 1.01 | 0.99 |
| | 100 | ng/ml | 1.05 | 0.95 | 0.99 | 0.88 | 0.93 | 1.12 | 1.05 | 0.95 | 0.93 | 0.90 | 0.97 | 1.11 |
| neuregulin 1 | 1.6 | ng/ml | 1.05 | 1.01 | 0.03 | 0.87 | 0.92 | 0.94 | 1.04 | 1.12 | 1.10 | 0.93 | 1.09 | 0.96 |
| | 6.25 | ng/ml | 1.07 | 1.07 | 0.98 | 1.06 | 1.18 | 0.91 | 1.08 | 1.20 | 1.04 | 0.96 | 1.08 | 0.97 |
| | 25 | ng/ml | 1.12 | 1.10 | 1.06 | 0.93 | 1.09 | 0.98 | 1.14 | 1.24 | 1.11 | 0.98 | 1.05 | 1.01 |
| | 100 | ng/ml | 1.09 | 1.20 | 1.24 | 0.99 | 1.09 | 1.04 | 1.18 | 1.52 | 1.37 | 1.01 | 1.08 | 1.06 |
| | 400 | ng/ml | 1.22 | 1.22 | 1.17 | 1.14 | 1.19 | 0.96 | 1.27 | 1.32 | 1.21 | 1.17 | 1.22 | 1.07 |
| NGF | 0.4 | ng/ml | 1.04 | 0.98 | 0.96 | 1.10 | 0.89 | 1.00 | 0.98 | 1.00 | 1.04 | 1.09 | 0.91 | 1.03 |
| | 1.6 | ng/ml | 1.02 | 0.98 | 0.96 | 0.87 | 0.97 | 1.03 | 0.95 | 1.07 | 1.03 | 0.78 | 0.92 | 1.04 |
| | 6.25 | ng/ml | 1.06 | 1.03 | 1.02 | 0.91 | 1.05 | 0.85 | 1.09 | 1.07 | 1.04 | 0.97 | 0.99 | 0.96 |
| | 25 | ng/ml | 1.07 | 0.97 | 0.96 | 0.92 | 1.00 | 0.93 | 0.99 | 0.98 | 0.94 | 0.85 | 0.96 | 1.07 |
| | 100 | ng/ml | 0.98 | 0.87 | 1.03 | 0.94 | 1.12 | 0.92 | 0.99 | 1.03 | 1.02 | 0.94 | 1.08 | 1.09 |
| NT3 | 0.4 | ng/ml | 1.02 | 1.00 | 1.03 | 0.95 | 0.95 | 0.99 | 1.04 | 0.98 | 1.07 | 0.98 | 1.02 | 1.02 |
| | 1.6 | ng/ml | 1.16 | 1.03 | 1.15 | 0.98 | 1.05 | 1.02 | 1.13 | 1.02 | 1.16 | 0.98 | 0.97 | 1.08 |
| | 6.25 | ng/ml | 1.09 | 0.91 | 1.00 | 1.00 | 0.89 | 0.93 | 1.08 | 0.93 | 1.02 | 1.01 | 0.99 | 1.00 |
| | 25 | ng/ml | 1.02 | 1.00 | 1.09 | 0.98 | 1.14 | 0.98 | 1.01 | 0.95 | 1.07 | 1.05 | 1.15 | 0.94 |
| | 100 | ng/ml | 0.93 | 0.95 | 1.04 | 0.84 | 0.94 | 1.09 | 0.90 | 0.88 | 1.03 | 0.88 | 0.96 | 1.04 |
| PDGF-BB | 0.4 | ng/ml | 1.07 | 0.96 | 1.01 | 0.93 | 1.03 | 0.87 | 1.07 | 1.06 | 1.04 | 1.00 | 1.10 | 1.03 |
| | 1.6 | ng/ml | 0.98 | 0.97 | 1.03 | 0.90 | 1.10 | 0.98 | 0.98 | 0.97 | 1.06 | 0.89 | 1.02 | 1.08 |
| | 6.25 | ng/ml | 1.07 | 1.04 | 1.06 | 0.98 | 1.16 | 1.02 | 1.10 | 1.08 | 1.10 | 0.99 | 1.05 | 1.04 |
| | 25 | ng/ml | 1.12 | 0.97 | 1.06 | 1.00 | 10.5 | 1.16 | 1.09 | 1.10 | 1.09 | 0.98 | 0.99 | 1.15 |
| | 100 | ng/ml | 1.07 | 0.89 | 1.01 | 1.00 | 1.13 | 1.31 | 1.02 | 0.96 | 0.98 | 0.96 | 1.03 | 1.16 |
| Pleiotrophin | 3.9 | ng/ml | 1.03 | 1.01 | 1.03 | 1.04 | 1.04 | 0.92 | 1.06 | 1.07 | 1.05 | 0.96 | 1.11 | 0.93 |
| | 15.625 | ng/ml | 1.04 | 1.04 | 0.96 | 0.97 | 0.95 | 0.89 | 1.07 | 1.08 | 0.99 | 0.96 | 0.98 | 1.00 |
| | 62.5 | ng/ml | 1.04 | 0.95 | 0.97 | 0.94 | 0.95 | 0.95 | 1.03 | 1.07 | 0.99 | 0.97 | 0.93 | 1.02 |
| | 250 | ng/ml | 1.02 | 0.96 | 0.36 | 0.94 | 0.96 | 1.07 | 1.00 | 1.12 | 0.96 | 0.92 | 1.02 | 1.08 |
| | 1000 | ng/ml | 0.93 | 0.87 | 0.98 | 1.00 | 0.89 | 0.97 | 0.87 | 0.99 | 0.95 | 0.92 | 0.87 | 1.11 |
| SCF | 0.4 | ng/ml | 1.04 | 1.05 | 0.96 | 1.07 | 0.92 | 0.90 | 1.04 | 1.02 | 1.00 | 1.06 | 1.03 | 0.97 |
| | 1.6 | ng/ml | 1.06 | 0.94 | 1.01 | 1.03 | 0.89 | 8.91 | 1.04 | 1.05 | 1.08 | 0.99 | 0.94 | 0.34 |
| | 6.25 | ng/ml | 1.07 | 0.97 | 1.06 | 1.04 | 0.98 | 0.96 | 1.13 | 1.03 | 1.15 | 1.06 | 1.01 | 1.02 |
| | 25 | ng/ml | 1.13 | 0.98 | 1.10 | 1.12 | 0.92 | 1.00 | 1.12 | 1.04 | 1.18 | 1.13 | 1.06 | 1.10 |
| | 100 | ng/ml | 1.08 | 0.96 | 1.04 | 1.07 | 0.98 | 1.09 | 1.09 | 0.98 | 1.09 | 1.07 | 0.99 | 1.12 |
| VEGF-A | 0.4 | ng/ml | 1.01 | 1.07 | 0.91 | 1.04 | 0.95 | 0.98 | 1.03 | 0.98 | 0.90 | 1.03 | 0.97 | 1.04 |
| | 1.6 | ng/ml | 0.98 | 0.96 | 0.93 | 1.01 | 0.96 | 0.81 | 0.96 | 0.99 | 0.93 | 0.90 | 0.92 | 0.36 |
| | 6.25 | ng/ml | 1.01 | 1.11 | 1.01 | 0.94 | 0.98 | 0.85 | 1.01 | 1.11 | 1.05 | 0.91 | 0.98 | 0.94 |
| | 25 | ng/ml | 1.06 | 1.03 | 0.95 | 0.97 | 1.05 | 1.08 | 1.02 | 1.10 | 0.95 | 0.89 | 0.88 | 1.09 |
| | 100 | ng/ml | 1.14 | 0.95 | 0.94 | 0.81 | 1.02 | 1.06 | 1.10 | 0.97 | 0.89 | 0.82 | 1.09 | 1.20 |
| VEGF-C | 1.6 | ng/ml | 1.03 | 0.94 | 0.99 | 1.01 | 1.05 | 0.95 | 1.01 | 1.06 | 0.97 | 0.97 | 1.09 | 0.97 |
| | 6.25 | ng/ml | 1.04 | 1.09 | 1.00 | 0.97 | 0.94 | 0.83 | 1.02 | 1.03 | 1.02 | 0.97 | 0.95 | 0.36 |
| | 25 | ng/ml | 1.05 | 1.06 | 0.92 | 0.92 | 0.98 | 0.87 | 1.01 | 1.16 | 0.94 | 0.91 | 0.90 | 0.96 |
| | 100 | ng/ml | 1.04 | 1.08 | 0.95 | 0.88 | 1.01 | 0.92 | 0.99 | 1.00 | 0.95 | 0.90 | 1.08 | 0.98 |
| | 400 | ng/ml | 1.02 | 1.08 | 0.90 | 0.85 | 0.89 | 0.85 | 0.94 | 1.07 | 0.90 | 0.89 | 0.85 | 0.33 |
| Wnt1 | 0.4 | ng/ml | 1.04 | 1.13 | 0.94 | 0.94 | 1.02 | 0.93 | 1.04 | 1.06 | 0.94 | 0.99 | 0.99 | 0.02 |
| | 1.6 | ng/ml | 1.00 | 1.00 | 0.92 | 0.97 | 1.25 | 0.90 | 0.99 | 0.96 | 0.93 | 1.00 | 1.18 | 0.91 |
| | 6.25 | ng/ml | 1.01 | 1.03 | 0.93 | 1.01 | 0.99 | 1.01 | 0.86 | 1.11 | 0.91 | 0.93 | 0.95 | 1.06 |
| | 25 | ng/ml | 0.98 | 1.01 | 0.06 | 0.97 | 1.15 | 0.97 | 0.98 | 1.02 | 0.85 | 0.96 | 1.18 | 1.02 |
| | 100 | ng/ml | 1.07 | 0.95 | 0.93 | 0.98 | 1.25 | 1.01 | 1.04 | 1.05 | 0.89 | 0.93 | 1.20 | 1.14 |

TABLE 5
Crizotinib synergistic effect

| | | | | Crizotinib | Crizotinib | Crizotinib | | |
|---|---|---|---|---|---|---|---|---|
| | | | DMSO | 0.125 | 0.25 | 0.5 | Crizotinib 1 | Crizotinib 2 |
| G361 | DMSO Control | No Fibro | 1.0 | 1.0 | 1.0 | 0.9 | 0.7 | 0.3 |
| | | 1069sk | 1.0 | 1.1 | 1.0 | 0.9 | 0.8 | 0.4 |
| | | HS-5 | 0.9 | 0.8 | 0.7 | 0.7 | 0.7 | 0.3 |
| | | LL86 | 1.4 | 1.2 | 1.1 | 1.1 | 0.8 | 0.4 |
| | | Wi-38 | 1.0 | 1.0 | 0.9 | 0.9 | 0.7 | 0.4 |
| | PLX4720 - 2 uM | No Fibro | 0.1 | 0.1 | 0.1 | 0.1 | 0.1 | 0.1 |
| | | 1069sk | 0.1 | 0.1 | 0.1 | 0.1 | 0.1 | 0.1 |
| | | HS-5 | 0.2 | 0.1 | 0.1 | 0.1 | 0.1 | 0.1 |
| | | LL86 | 0.5 | 0.1 | 0.1 | 0.1 | 0.1 | 0.1 |
| | | Wi-38 | 0.4 | 0.1 | 0.1 | 0.1 | 0.1 | 0.1 |
| SK-MEL-28 | DMSO Control | No Fibro | 1.0 | 1.0 | 1.1 | 1.0 | 0.8 | 0.6 |
| | | 1069sk | 1.0 | 0.9 | 1.0 | 0.9 | 0.8 | 0.6 |
| | | HS-5 | 1.0 | 1.0 | 0.9 | 0.9 | 0.7 | 0.6 |
| | | LL86 | 1.0 | 1.0 | 1.0 | 1.0 | 0.8 | 0.6 |
| | | Wi-38 | 1.0 | 1.0 | 1.0 | 0.9 | 0.9 | 0.6 |
| | PLX4720 - 2 uM | No Fibro | 0.1 | 0.1 | 0.1 | 0.1 | 0.1 | 0.1 |
| | | 1069sk | 0.1 | 0.1 | 0.1 | 0.1 | 0.1 | 0.1 |
| | | HS-5 | 0.1 | 0.1 | 0.1 | 0.1 | 0.1 | 0.1 |
| | | LL86 | 0.3 | 0.1 | 0.1 | 0.1 | 0.1 | 0.1 |
| | | Wi-38 | 0.4 | 0.1 | 0.1 | 0.1 | 0.1 | 0.1 |
| SK-MEL-5 | DMSO Control | No Fibro | 1.0 | 1.0 | 0.9 | 0.8 | 0.6 | 0.5 |
| | | 1069sk | 1.1 | 1.1 | 1.1 | 1.0 | 0.8 | 0.5 |
| | | HS-5 | 0.9 | 0.9 | 0.9 | 0.8 | 0.6 | 0.4 |
| | | LL86 | 1.0 | 1.0 | 1.0 | 0.9 | 0.7 | 0.6 |
| | | Wi-38 | 1.0 | 0.9 | 0.9 | 0.8 | 0.6 | 0.5 |
| | PLX4720 - 2 uM | No Fibro | 0.3 | 0.3 | 0.3 | 0.3 | 0.2 | 0.2 |
| | | 1069sk | 0.4 | 0.4 | 0.3 | 0.3 | 0.3 | 0.2 |
| | | HS-5 | 0.5 | 0.5 | 0.5 | 0.4 | 0.3 | 0.2 |
| | | LL86 | 0.8 | 0.4 | 0.3 | 0.3 | 0.2 | 0.2 |
| | | Wi-38 | 0.6 | 0.3 | 0.3 | 0.3 | 0.2 | 0.2 |

TABLE 6
RTKs expression in melanoma cell lines and their ligands

| | RTK | | | | | | | | | |
|---|---|---|---|---|---|---|---|---|---|---|
| RTK | Class | A2058 | C32 | COLO829 | G361 | SKMEL28 | SKMEL5 | A375 | MALME3M | Ligand |
| EGFR | 1 | | | | | | | 106.2 | | EGF |
| ERBB3 | 1 | 4688.4 | 3895.4 | 3065.6 | 1689.2 | 1382.5 | 3194.5 | 5015.5 | 5694.7 | Neuroregulin |
| ERBB4 | 1 | | 30.8 | | | | 13.0 | | 15.1 | Neuroregulin |
| IGF1R | 2 | 585.2 | 1167.3 | 1248.6 | 1580.1 | 1302.6 | 689.2 | 954.5 | 1016.8 | IGF-1 |
| INSR | 2 | | 215.3 | 318.3 | 113.8 | 152.0 | 152.8 | 176.4 | 107.5 | Insulin |
| KIT | 3 | | | | 51.0 | | | | 988.8 | SCF |
| CSF1R | 3 | 19.5 | | | 17.4 | | | | 18.3 | M-CSF |
| PDGFRA | 3 | 17.6 | | | | | | 19.7 | | PDGF-BB |
| PDGFRB | 3 | | | | | | | | | PDGF-BB |
| FGFR1 | 4 | 97.1 | 140.9 | 129.1 | 103.9 | 134.6 | 224.1 | 162.8 | 245.5 | FGF-1 |
| FGFR2 | 4 | 55.7 | 56.1 | 42.4 | 85.3 | 53.2 | 42.1 | 38.0 | 43.1 | FGF-1 |
| FGFR3 | 4 | 77.1 | 206.3 | | | 38.9 | | 97.9 | | FGF-1 |
| FLT1 | 5 | 87.2 | 114.0 | 180.8 | 76.3 | 100.4 | 98.2 | 113.2 | 74.4 | VEGF-A |
| KDR | 5 | 24.1 | 297.2 | 897.5 | 44.7 | 17.3 | 64.4 | 33.4 | 54.8 | VEGF-A |
| FLT4 | 5 | | | | | | | | | VEGF-C |
| MET | 6 | 1193.7 | 1458.6 | 4325.5 | 1686.0 | 686.6 | 2309.2 | 1132.7 | 1721.2 | HGF |
| MST1R | 6 | | | | | | | | | MSP |
| NTRK1 | 7 | 18.2 | | | | | | | | NGF |
| NTRK2 | 7 | 29.3 | 40.3 | 31.9 | | | | | 18.8 | BDNF |
| NTRK3 | 7 | | | 13.8 | | | | | 25.4 | NT3 |
| EPHA2 | 8 | 268.4 | | 135.5 | | | | 674.6 | 136.0 | Ephrin-A |
| EPHA3 | 8 | 713.8 | 291.7 | 193.4 | 389.3 | 34.5 | 583.4 | 600.5 | 83.3 | Ephrin-A |
| EPHA4 | 8 | 337.2 | | 14.4 | 518.9 | 377.0 | 219.6 | 95.0 | 291.8 | Ephrin-A |
| AXL | 9 | 1643.7 | 196.5 | 1629.6 | 44.5 | 46.7 | | 3030.2 | 1264.3 | Gas6 |
| MERTK | 9 | 38.0 | 20.4 | 110.9 | 1116.4 | 83.3 | 1803.9 | | 544.4 | Gas6 |
| TYRO3 | 9 | 248.8 | 157.8 | 195.1 | 375.8 | 181.6 | 574.3 | 239.7 | 307.7 | Gas6 |
| ALK | 10 | 31.5 | 54.3 | 30.0 | | 51.3 | 23.3 | | 59.6 | pleiotrophin |
| ROR1 | 12 | 203.4 | 52.5 | 85.5 | 105.7 | 96.6 | 74.2 | 48.5 | 85.3 | Wnt1 |
| ROR2 | 12 | | | | | | | | | Wnt1 |
| DDR1 | 13 | 1264.7 | 1161.7 | 1128.8 | 590.1 | 126.0 | 665.7 | 746.0 | 1153.4 | Type II |
| | | | | | | | | | | colagen |
| DDR2 | 13 | 612.3 | 939.5 | 393.9 | 71.3 | 400.5 | 249.3 | 689.6 | 183.2 | Type II |
| | | | | | | | | | | colagen |
| RET | 14 | 14.1 | | | | | | | | GDNF |
| RYK | 16 | 670.7 | 915.4 | 892.3 | 860.5 | 1495.4 | 634.5 | 1010.5 | 699.6 | Wnt1 |

TABLE 7
Clinical Data

| Patient | | Site of | Site of Biopsy | Site of Biopsy |
|---|---|---|---|---|
| # | Age | disease | (Pre-Tx) | (On-Tx) |
| 1 | 56 | sc, n | Skin (SC nodule) | Skin (SC nodule) |
| 2 | 54 | li, lu | Hepatectomy | |
| 3 | 72 | sc, br | Skin (SC nodule) | Skin (SC nodule) |
| 4 | 47 | n, sc | Mid lower abd (SC) | |
| 5 | 69 | n, li, lu | Lymph node | |
| 6 | 49 | n, lu | Lymph node | |
| 7 | 82 | n, li | Hepatectomy | |
| 8 | 79 | n, sc | Lymph node | |
| 9 | 64 | n, sc | Skin (SC nodule) | |
| 10 | 48 | sk, n | Right Axillary node | |
| 11 | 43 | sc, n | Skin (SC nodule) | Skin (SC nodule) |
| 12 | 57 | sk | skin | |
| 13 | 31 | sc, br, n | Skin (SC nodule) | Skin (SC nodule) |
| 14 | 65 | sk, sc, lu, br | Left flank/buttock (SC) | |
| 15 | 69 | sc, n | Skin (SC nodule) | Skin (SC nodule) |
| 16 | 65 | n, li | Left lower abd (cutaneous) | |
| 17 | 36 | n, br | nodal | nodal |
| 18 | 37 | sc, br | Skin (SC nodule) | Skin (SC nodule) |
| 19 | 68 | sc | Skin (SC nodule) | Skin (SC nodule) |
| 20 | 42 | n, sc, br, lu | Skeletal muscle | |
| 21 | 65 | n, sc, lu, m | Right chest (SC) | |
| 22 | 37 | li, sc, n | Skin (SC nodule) | Skin (SC nodule) |
| 23 | 42 | sc | Skin (SC nodule) | Skin (SC nodule) |
| 24 | 67 | lu, b, st | skin | |
| 25 | 68 | n | nodal | nodal |
| 26 | 54 | n, li, lu | small intestine | |
| 27 | 73 | sc | Skin (SC nodule) | Skin (SC nodule) |
| 28 | 61 | n, sc, lu, li, ad | Left scalp (SC) | |
| 29 | 74 | sk | Right ant thigh cutaneous | |
| 30 | 44 | li, sb | Small bowel | |
| 31 | 61 | br, lu, n | skin | |
| 32 | 49 | lu, sc | Skin (SC nodule) | Skin (SC nodule) |
| 33 | 74 | n | nodal | nodal |
| 34 | 77 | n, lu | Left base of neck (SC) | |

sk = skin sc = subcutaneous n = nodal lu = lung li = liver
br = brain b = bone m = muscle ad = adrenal sb = small bowel,
st = soft tissue, sp = spleen

| | | | | | Time to |
|---|---|---|---|---|---|
| Patient | | | Response | Response | progression |
| # | Treatment | Specific drug | (Max) | (Max) | (months) |
| 1 | BRAFi + MEKi | GSK2118436 + GSK1123212 | 100% | CR | 14, Ongoing |
| 2 | BRAFi | Vemurafenib | 90% | PR | 7 |
| 3 | BRAFi + MEKi | GSK2118436 + GSK1123212 | 76% | PR | 10 |
| 4 | BRAFi | Vemurafenib | 72% | PR | 3.2 |
| 5 | BRAFi + MEKi | GSK2118436 + GSK1123212 | 63% | PR | 13 |
| 6 | BRAFi + MEKi | GSK2118436 + GSK1123212 | 60% | PR | 12, Ongoing |
| 7 | BRAFi | Vemurafenib | 50% | PR | 5 |
| 8 | BRAFi + MEKi | GSK2118436 + GSK1123212 | 33% | PR | 5 |
| 9 | BRAFi + MEKi | GSK2118436 + GSK1123212 | 23% | SD | 3 |
| 10 | BRAFi | GSK2118436 | 9% | SD | 3.5 |
| 11 | BRAFi + MEKi | GSK2118436 + GSK1123212 | 5.7% | SD | 7, Ongoing |
| 12 | BRAFi | Vemurafenib | 100% | CR | 3 |
| 13 | BRAFi + MEKi | GSK2118436 + GSK1123212 | 86% | PR | 9, Ongoing |
| 14 | BRAFi | Vemurafenib | 72% | PR | 12.4 |
| 15 | BRAFi + MEKi | GSK2118436 + GSK1123212 | 42% | PR | 9, Ongoing |
| 16 | BRAFi | GSK2118436 | 40% | PR | 2.5 |
| 17 | BRAFi + MEKi | GSK2118436 + GSK1123212 | 37% | PR | 9 |
| 18 | BRAFi + MEKi | GSK2118436 + GSK1123212 | 30% | PR | 5.5 |
| 19 | BRAFi | Vemurafenib | 25% | SD | 5 |
| 20 | BRAFi | Vemurafenib | 23% | SD | 8 |
| 21 | BRAFi | Vemurafenib | 22% | SD | 5.4 |
| 22 | BRAFi + MEKi | GSK2118436 + GSK1123212 | 13% | SD | 3 |
| 23 | BRAFi | Vemurafenib | 10% | SD | 4.5 |
| 24 | BRAFi | Vemurafenib | 0% | POD | |
| 25 | BRAFi | Vemurafenib | 54% | PR | 8.5 |
| 26 | BRAFi + MEKi | GSK2118436 + GSK1123212 | 50% | PR | 4 |
| 27 | BRAFi + MEKi | GSK2118436 + GSK1123212 | 38% | PR | 15, Ongoing |
| 28 | BRAFi | Vemurafenib | 34% | PR | 6.0 |
| 29 | BRAFi | Vemurafenib | 20% | SD | 7.3 |
| 30 | BRAFi | Vemurafenib | 0% | POD | |
| 31 | BRAFi | Vemurafenib | 0% | POD | |
| 32 | BRAFi | Vemurafenib | 56% | PR | 3.5 |
| 33 | BRAFi | Vemurafenib | 27% | SD | 6.5 |
| 34 | BRAFi | Vemurafenib | 0% | POD | 1.3 |

PR = partial response CR = complete response
SD = stable disease POD = progression of disease

| | Pre Treatment | | | | | | On-Tx | | |
|---|---|---|---|---|---|---|---|---|---|
| Patient | Tumor | Stromal | Tumor | | | | Tumor | Stromal | Tumor |
| # | HGF | HGF | MET | pERK | pAKT | pMET | HGF | HGF | MET |
| 1 | 0 | 0 | | 3 | 1 | 0 | (*1) | 0 | |
| 2 | 0 | 0 | 1 | | | | (*1) | | |
| 3 | 1 | 0 | | 2 | 2 | | 1 | 1 | |
| 4 | 1 | 0 | | | | | | | |
| 5 | 1 | 0 | 2 | | | | (*2) | | |
| 6 | 1 | 0 | 1 | | | | | | |
| 7 | 0 | 0 | 2 | | | | | | |
| 8 | 1 | 0 | 2 | | | | (*3) | | |
| 9 | 1 | 0 | 1 | | | | | | |
| 10 | 0 | 0 | | | | | | | |
| 11 | 1 | 0 | | 3 | 3 | | 1 | 1 | |
| 12 | 1 | 1 | 1 | | | | | | |
| 13 | 0 | 1 | | 3 | 1 | | 0 | 1 | |
| 14 | 1 | 1 | | | | | | | |
| 15 | 0 | 1 | | 3 | 2 | | (*1) | (*2) | |
| 16 | 1 | 1 | | | | | | | |
| 17 | 0 | 1 | | 3 | 2 | | (*3) | (*2) | |
| 18 | 1 | 1 | | 3 | 3 | 2 | 1 | 2 | |
| 19 | 1 | 1 | | 2 | 2 | | 0 | 2 | |
| 20 | (*4) | 1 | (*4) | | | | | | |
| 21 | 1 | 1 | | | | | | | |
| 22 | 0 | 1 | | 3 | 2 | | (*4) | (*4) | |
| 23 | 0 | 1 | | 1 | 1 | | (*1) | 2 | |
| 24 | 1 | 1 | 3 | | | | | | |
| 25 | 2 | 2 | 1 | 3 | 3 | 1 | 2 | 2 | 2 |
| 26 | 0 | 2 | 0 | | | | | | |
| 27 | 1 | 2 | 2 | 2 | 2 | 2 | 0 | 2 | 2 |
| 28 | 2 | 2 | | | | | | | |
| 29 | 2 | 2 | | | | | | | |
| 30 | 1 | 2 | 0 | | | | | | |
| 31 | 0 | 2 | 1 | | | | | | |
| 32 | 0 | 3 | | | | | 0 | (*2) | |
| 33 | 1 | 3 | 2 | 2 | 0 | | (*1) | 3 | (*1) |
| 34 | 1 | 3 | | | | | | | |

| | | | | Progression | | | | |
|---|---|---|---|---|---|---|---|---|
| Patient | On-Tx | | | Tumor | Stromal | Tumor | | |
| # | pERK | pAKT | pMET | HGF | HGF | MET | pERK | pAKT |
| 1 | | | 0 | | | | | |
| 2 | | | | | | | | |
| 3 | | | | | | | | |
| 4 | | | | | | | | |
| 5 | | | | | | | | |
| 6 | | | | | | | | |
| 7 | | | | | | | | |
| 8 | | | | | | | | |
| 9 | | | | | | | | |
| 10 | | | | | | | | |
| 11 | 1 | 2 | | | | | | |
| 12 | | | | | | | | |
| 13 | | | | | | | | |
| 14 | | | | | | | | |
| 15 | | | | | | | | |
| 16 | | | | | | | | |
| 17 | | | | 3 | 0 | | 3 | |
| 18 | 2 | 2 | 2 | | | | | |
| 19 | 1 | | | | | | | |
| 20 | | | | | | | | |
| 21 | | | | | | | | |
| 22 | | | | | | | | |
| 23 | | | | 0 | 3 | | 2 | 2 |
| 24 | | | | | | | | |
| 25 | 2 | 1 | 2 | | | | | |
| 26 | | | | | | | | |
| 27 | 0 | 3 | 2 | | | | | |
| 28 | | | | | | | | |
| 29 | | | | | | | | |
| 30 | | | | | | | | |
| 31 | | | | | | | | |
| 32 | | | | | | | | |
| 33 | | | | | | | | |
| 34 | | | | | | | | |

(*1) = Tumor cells undetectable or very few (*2) = Stromal cells undetectable
or very few (*3) = Necrotic tissue/Non-specific staining (*4) = Unable to
evaluate because of abundant melanin
For tumor HGF, MET, p-AKT, p-ERK and pMET, staining intensity was
recorded as no expression (score 0), weak expression (score 1), moderate
expression (score 2), or strong expression (score 3).
For stromal HGF, staining intensity in stromal fibroblasts adjacent to tumor
cells was recorded as above. For statistical analysis: positive stromal HGF
was defined when some stromal HGF was present (score 1 to 3), while negative
stromal HGF was defined when no stromal HGF was present (score 0).

1. A method of preventing or reducing chemoresistance in a tumor comprising administering to a subject with cancer a chemotherapeutic agent and a c-MET kinase (MET) inhibitor.

2. The method of claim 1, wherein the chemoresistance is stromal cell mediated.

3. The method of claim 1, wherein the tumor comprises a B-RAF activating mutation.

4. A method of treating a tumor having a B-RAF activating mutation in a subject comprising administering an effective amount of a MET inhibitor to the subject.

5. The method of claim 4, where said subject has been previously exposed to one or more chemotherapeutic agents.

6. The method of claim 5, wherein said tumor is refractory to chemotherapeutic agent;

7. The method of claim 1, wherein the tumor is a melanoma, colon cancer, lung cancer, brain cancer, thyroid cancer or a hematologic cancer.

8. The method of claim 1, wherein the chemotherapeutic agent is a B-RAF inhibitor, a MEK inhibitor, a PI3K inhibitor, an AKT inhibitor or a combination thereof.

9. The method of claim 8, wherein said B-RAF inhibitor is Vemurafenib or Dabrafenib.

10. The method of claim 1, wherein the MET inhibitor is a small molecule, a hepatocyte growth factor (HGF) neutralizing antibody or a MET neutralizing antibody.

11. The method of claim 1, wherein the MET inhibitor is (3Z)-5-(2,3-dihydro-1H-indol-1-ylsulfonyl)-3-({3,5-dimethyl-4-[(4-methylpiperazin-1-yl)carbonyl]-1H-pyrrol-2-yl}methylene)-1,3-dihydro-2H-indol-2-one, (3Z)-N-(3-chlorophenyl)-3-({3,5-dimethyl-4-[(4-methylpiperazin-1-yl)carbonyl]-1H-pyrrol-2-yl}methylene)-N-methyl-2-oxoindoline-5-sulfonamide, (3Z)-N-(3-chlorophenyl)-3-{[3,5-dimethyl-4-(3-morpholin-4-ylpropyl)-1H-pyrrol-2-yl]methylene}-N-methyl-2-oxoindoline-5-sulfonamide, AMG-208, AMG-337, Axitinib, Foretinib, JNJ-38877605, MGCD-265, PF-04217903, Crizotinib, Cabozantinib, PHA-665752, SGX-523, SU11274, XL184, ARQ197, XL880, INC280 Onartuzumab, Trametinib, selumetinib, PD0325901, PD184,352, PHA-665752, JNJ-38877605, Rilotumumab or Ficlatuzumab.

12. The method of claim 1, wherein the MET inhibitor is administered concurrently with one or more chemotherapeutic agents

13. The method of claim 1, wherein the MET inhibitor is administered prior to readministration of the one or more chemotherapeutic agents.

14. The method of claim 1, wherein the MET inhibitor is administered into or near the tumor.

15. The method of claim 1, wherein the MET inhibitor is administered systemically.

16. A method of diagnosing or determining a predisposition to developing chemoresistance in a tumor comprising determining the level of HGF in expression in the tumor and comparing the level of HGF expression to a control sample, wherein an increase level of HGF expression in the tumor compared to the control indicates chemoresistance or a predisposition to developing chemoresistance in the tumor.

17. A method of diagnosing or determining a predisposition to developing chemoresistance in a tumor comprising determining the level of MET activation in the tumor and comparing the level of MET activation to a control sample, wherein an increase level of MET activation in the tumor compared to the control indicates chemoresistance or a predisposition to developing chemoresistance in the tumor.

18. The method of claim 16, wherein in the tumor has a BRAF activating mutation.

19. The method of claim 16, wherein said levels of HGF expression is determined by detecting HGF polypeptide, or HGF nucleic acid.

20. The method of claim 19, wherein the nucleic acid is RNA or DNA.

21. The method of claim 17, wherein MET activation is determined by detecting MET phosphorylation.
